(19) United States
(12) Patent Application Publication
(10) Pub. No.: US 2017/0282170 A9
(43) Pub. Date: Oct. 5, 2017

(54) PHOSPHORUS-CONTAINING CATALYSTS

(71) Applicant: AGENCY FOR SCIENCE, TECHNOLOGY AND RESEARCH, Singapore (SG)

(72) Inventors: Charles W. Johannes, Singapore (SG); Edward G. Robins, Singapore (SG); Howard Jong, Singapore (SG); Yee Hwee Lim, Singapore (SG); Saei Weng Chia, Singapore (SG); Yong Yang, Singapore (SG); Anil Podichetty, Singapore (SG)

(73) Assignee: Agency for Science, Technology and Research, Singapore (SG)

(21) Appl. No.: 14/913,479

(22) PCT Filed: Aug. 29, 2014
(86) PCT No.: PCT/SG2014/000413

§ 371 Date: Feb. 22, 2016

(30) Foreign Application Priority Data
Aug. 29, 2013 (SG) 2013065339

(51) Int. Cl. B01J 31/24; C07C 2/86; C07F 15/00; C07C 17/361; C07D 209/10; C07D 409/04; C07D 295/033; C07C 37/18; B01J 31/18; C07F 9/50

(52) U.S. Cl. CPC B01J 31/2409; B01J 31/2404; B01J 31/189; B01J 31/1875; B01J 31/2495; C07C 2/861; C07F 15/0066; C07F 15/006; C07F 9/5063; C07D 209/10; C07D 409/04; C07D 295/033; C07C 37/18; C07C 17/361; B01J 2231/4261; B01J 2231/4227; B01J 2231/4283; B01J 2231/4266; B01J 2231/4233; B01J 2231/40; B01J 2531/824

(57) ABSTRACT

The invention provides compounds of general structure I: (Ar¹—Ar²—Ar³-E-P(=D)R₂-)ₙMₘXₙLₙ″. In this structure: •Ar¹, Ar² and Ar³ are aromatic groups wherein: —Ar¹ and Ar³ are in a 1,3 relationship on Ar², —each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent or is O or S, 2 if Y is B or N and 3 if Y is Si, —Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms; •E is absent or is selected from the group consisting of O, S, NR″, SiR″₂, AsR″₂ and CR″₂; •M is a complexing metal; •X is selected from the group consisting of H, F, Br, CI, I, OTf, dba (dibenzylidene acetone), OC(═O)CF₃ and OAc; •L is selected from the group consisting of PR″₂, NR″₂, OR″, SR″, SiR″₃, AsR″₃, alkene, alkyne, aryl and heteroaryl, each of said alkene, alkyne, aryl and heteroaryl being optionally substituted, for example with one or more halogens and/or with one or more R groups as defined herein; •each R is independently alkyl, cycloalkyl, heterocyclyl, heterocycloalkyl, aryl or -, heteroaryl; •D is absent or is ═S or —O or —Z-linker-Z—, where each Z independently is O or NH or N-alkyl and linker is an alkyl chain of 2-5 carbon atoms in length; •each R″ is independently H, alkyl, cycloalkyl, heterocycloalkyl, aryl or heteroaryl, each other than H being optionally substituted, or R″₂ is —Z-linker-Z— as defined above; and •m is 0 or 1 or 2; wherein if m is 0, n is 1, n′ and n″ are 0 and -- is absent; and if m is 1 or 2, n is 1 or 2 and n′ and n″ are integers such that the coordination sphere of M is filled, and D is absent.

### FIELD

This application relates to novel phosphorus containing catalysts, to processes for making them and for methods of using them.

### PRIORITY

This application claims priority from Singapore application SG201306533-9 filed 29 Aug. 2013, the entire contents of which are incorporated herein by cross-reference.

### BACKGROUND

Palladium catalysts are amongst the most well known class of catalysts in all of synthetic chemistry. They have wide-spread application in both academia and in industry where they efficiently facilitate the preparation of fine chemicals that are consumed by the pharmaceutical, polymer and agrochemicals sectors. The sources of their success reside in their activity, atom economy, selectivity, broad-based applicability and tolerance of various functional groups. As such, there has been a tremendous amount of effort from both industrial and academic groups to research and exploit the vast potential of palladium catalysts that typically contain phosphine ligands—most of which was established by empirical success.

While there is no doubt of the exceptional usefulness of phosphine ligands for metal-mediated catalysis, there are examples where phosphine ligands are not the best in class for selected catalytic transformations. One such example is the Suzuki-Miyaura cross-coupling reaction where ortho-palladated arylphosphinite complexes have been shown to surpass the activity of their commonly used Pd-phosphine congeners for a variety of substrates. However, despite the high activity of Pd-phosphinite catalysts, there has not been significant adoption in industry as the process to optimize and showcase broad-based applicability with global recognition throughout the research community is slow. Notwithstanding, this trend is changing as the benefits (i.e. ease of synthesis, catalytic activity, robustness and broad substrate scope) of employing phosphinite, phosphonite and phosphite catalysts are emerging with momentum.

More recently, particular attention has been aimed at catalyst activity in industry as the inflationary trend of precious metal commodities is a growing concern. In the past, precious metal catalysts were only deemed viable for industrial applications if turnover numbers (TONs) in excess of 10,000 could be achieved. With increasing commodity prices, the performance benchmarks for precious metal catalysts are also rising as companies manage their expenses and margins. Furthermore, as organizations move to greener practices, process improvement efforts to lower the metal content, reduce wastage and improve atom economy in reactions have become key areas of focus to comply with global sustainability initiatives.

Despite the catalysis research community trending towards developing greener and more productive systems, the need to invent highly active catalysts for challenging reactions and new transformations will always remain high. With a perpetually growing list of new catalysts that are capable of performing well known reactions, it is becoming increasingly difficult for scientists, both at the industrial and academic level, to identify which systems are the most suitable for their application. It is widely acknowledged that examples of catalysts that are highly effective for a multitude of applications are very limited. Thus, improvements to these versatile catalyst systems would be of tremendous value to the research community as they would aliviate the challenging of catalyst selection and improve the synthetic efficiency of functional molecules by being able to perform a sequence of different catalytic reactions in a single pot, or under continuous flow conditions.

### SUMMARY OF INVENTION

Aspects of the invention may be described by the following statements

1. A process for making a compound of structure I:

(Ar¹—Ar²—Ar³-E-PR₂—-)ₙMₘX_{n′}L_{n″}

wherein:
Ar¹, Ar² and Ar³ are aromatic groups wherein:
   Ar¹ and Ar³ are in a 1,3 relationship on Ar²,
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, heterocyclyl, cycloheteroaryl, aryl or heteroaryl and r is 1, 2 or 3, wherein r is 1 if Y is absent or is O or S, 2 if Y is B or N and 3 if Y is Si,
   Ar¹, Ar² and Ar³ each is independently carbocyclic or heterocyclic and each is independently monocyclic, bicyclic or polycyclic and each ring of each of Ar¹, Ar² and Ar³ independently has 5, 6 or 7 ring atoms;
E is absent or is selected from the group consisting of O, S, NR″, SiR″₂, AsR″₂ and CR″₂;
M is a complexing metal;
X is selected from the group consisting of H, F, Br, Cl, I, OTf, dba (dibenzylidene acetone), OC(═O)CF₃ and OAc;
L is selected from the group consisting of PR″₃, P(OR″)₃, NR″₃, OR″, SR″, SiR″₃, AsR″₃, alkene, alkyne, aryl and heteroaryl, each of said alkene, alkyne, aryl and heteroaryl being optionally substituted, for example with one or more halogens and/or with one or more R groups as defined herein;
each R is independently alkyl, cycloalkyl, heterocyclyl, heterocycloalkyl, aryl or heteroaryl, each being optionally substituted with one or more halogens and/or alkyl groups and/or aryl groups;
each R″ is independently H, alkyl, cycloalkyl, heterocyclyl, heterocycloalkyl aryl or heteroaryl, each other than H being optionally substituted with one or more halogens and/or alkyl groups and/or aryl groups, or R″₂ is —Z-linker-Z— where each Z independently is O or NH or N-alkyl and linker is an alkyl chain of 2-5 carbon atoms in length; and
m is 1 or 2, n is 1 or 2 and n′ and n″ are integers such that the coordination sphere of M is filled;
   said process comprising reacting a compound of structure II:

Ar¹—Ar²—Ar³-E-PR₂

wherein Ar¹, Ar² and Ar³, E and R are as defined above and Ar¹ and Ar³ are in a 1,3 relationship on Ar²,
with a salt or complex of M, optionally in the presence of a liganding species.

2. The process of statement 1 wherein no liganding species is present, whereby m=2, said process comprising the subsequent step of exposing the compound of structure I in which m=2 to a liganding species so as to produce a compound of structure I in which m=1 and L is said liganding species or is derived therefrom.

3. The process of statement 1 or statement 2 wherein the liganding species is a phosphine, an amine, an alcohol, a thiol, a silane, an arsine, an olefin, an aromatic compound or a heteroaromatic compound.

4. The process of any one of statements 1 to 3 wherein M is selected from the group consisting of Pd, Ni, Pt, Cu, Fe, Co, Au, Ag, Rh, Ir, Ru, Os and Mn.

5. The process of any one of statements 1 to 4 wherein the salt or complex of M is a dba, diene, olefin, allyl, silane, nitrile or organonitrile complex (such as Pd(PhCN)₂Cl₂, Pd(CN)₂ or Pd(MeCN)₄(BF₄)₂ or Pd(COD)(CH₂TMS)₂ or Pd(NBD)(CH₂TMS)₂), or a halide or acetate or cyanide salt or a salt of a pseudohalide, e.g. triflate, of M.

6. The process of statement 4 wherein M is Pd, Ni or Cu.

7. The process of statement 6 wherein M is Pd.

8. A process for making a compound of structure II as defined in statement 1 comprising reacting a compound of structure III:

Ar¹—Ar²—Ar³-E-X

wherein Ar¹, Ar² and Ar³ and E are as defined above and Ar¹ and Ar³ are in a 1,3 relationship on Ar²,
with either:
a compound of structure X—PR₂, wherein R is as defined in statement 1 or
a compound of structure X—P(D)R₂ wherein R is as defined in statement 1, and subsequently removing the group D, wherein D is ═S or ═O or —Z-linker-Z—, where each Z independently is O or NH or N-alkyl and linker is an alkyl chain of 2-5 carbon atoms in length
wherein each X is independently selected from the group consisting of H, F, Br, Cl, I, OTf, dba, OC(═O)CF₃ and OAc.

9. The process of any one of statements 1 to 8 wherein each ring in Ar¹, Ar² and Ar³ has independently, 5 or 6 ring carbon atoms.

10. The process of any one of statements 1 to 9 wherein at least one of Ar¹, Ar² and Ar³ is a fused aromatic ring or a fused heteroaryl ring.

11. The process of any one of statements 1 to 9 wherein each of Ar¹, Ar² and Ar³ is monocyclic, e.g. is a phenyl ring.

12. The process of any one of statements 1 to 11 wherein E is O, S, NR″₂ or absent.

13. The process of any one of statements 1 to 12 wherein the R groups on P are both alkyl or both are aryl or one R is alkyl and one is aryl.

14. The process of statement 8 comprising the step of conducting a metal mediated cross-coupling reaction of a biaryl compound with an aryl compound so as to produce the compound of structure III.

15. The process of statement 14 comprising reacting a compound of structure IV: Ar¹—Ar²A in which A is a leaving group or activating group, e.g. a halogen or a triflate group or a boronic acid or boronic ester, and in which A is meta to Ar¹ on Ar², with a compound of structure XEAr³A or Ar³A in the presence of an electrophile, so as to produce the compound of structure III.

16. The process of statement 15 wherein A is either an electron donating group or an electron withdrawing group.

17. The process of statement 1 comprising:
providing a 2,4,6-trialkylbiphenyl
halogenating said 2,4,6-trialkylbiphenyl in the 3 position
metallating the resulting halogenated biphenyl and reacting the resulting intermediate with an o-halophenol to form a 2-hydroxy-2′,4′,6′-trialkyl-m-terphenyl, or with an o-dihalobenzene to form a 2-halo-2′,4′,6′-trialkyl-m-terphenyl, and
reacting the m-terphenyl with a base and a halodialkylphosphine or halodiarylphosphine or haloalkylarylphosphine to produce a dialkylphosphinite or diarylphosphinite or alkylarylphosphinite of 2-hydroxy-2′,4′,6′-trialkyl-m-terphenyl respectively or else metallating the m-terphenyl and reacting the resulting intermediate, optionally in the presence of a copper salt and/or lithium salt, and a halodialkylphosphine or halodiarylphosphine or haloalkylarylphosphine to produce a dialkylphosphine or diarylphosphine or alkylarylphosphine respectively, each of said dialkylphosphinite or diarylphosphinite or alkylarylphosphinite or dialkylphosphine or diarylphosphine or alkylarylphosphine being a compound of structure II.

18. The process of statement 17 wherein, in the step of metallating, the intermediate is reacted with an o-halophenol and the subsequent step comprises reacting the resulting m-terphenyl with a base and a halodialkylphosphine or halodiarylphosphine or haloalkylarylphosphine to produce a dialkylphosphinite or diarylphosphinite or alkylarylphosphiniteof 2-hydroxy-2′,4′,6′-trialkyl-m-terphenyl respectively, each being of structure II in which E is O.

19. The process of statement 18 comprising the subsequent step of reacting the compound of structure II with a palladium halide so as to form a compound of structure I as defined in statement 1 in which E is O, m, n and n′ are all 2, n″ is 0 and both R groups are alkyl or both are aryl or one R is alkyl and the other is aryl.

20. The process of statement 18 comprising reacting the compound of structure II with a palladium halide in the presence of a phosphine ligand whereby the process forms a compound of structure I as defined in statement 1 in which E is O, m, n and n′ are all 1, n″ is 1 and both R groups are alkyl or both are aryl or one R is alkyl and the other is aryl and L is the phosphine ligand.

21. The process of statement 19 comprising the subsequent step of reacting the compound of structure I with a phosphine ligand, e.g. PCy₃, so as to produce a compound of structure I as defined in statement 1 in which E is O, m, n and n′ are all 1, n″ is 1 and both R groups are alkyl or both are aryl or one R is alkyl and the other is aryl and L is the phosphine ligand, e.g. PCy₃.

22. The process of any one of statements 1 to 21 which is conducted as a one pot process without isolation of intermediates or in which any two or more contiguous steps are conducted as a one pot process.

23. The process of any one of statements 1 to 22 which is conducted as a continuous reaction or in which any one or more individual steps thereof is (are) conducted as a continuous reaction.

24. A compound of structure I:

(Ar¹—Ar²—Ar³-E-P(=D)R₂—-)ₙMₘX_{n′}L_{n″}

wherein:
Ar¹, Ar² and Ar³ are aromatic groups wherein:
   Ar¹ and Ar³ are in a 1,3 relationship on Ar²,
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is O or S, 2 if Y is B or N and 3 if Y is Si,
   Ar¹, Ar² and Ar³ is each independently carbocyclic or heterocyclic and each is independently monocyclic, bicyclic or polycyclic and each ring of each of Ar¹, Ar² and Ar³ independently has 5, 6 or 7 ring atoms;
E is absent or is selected from the group consisting of O, S, NR″, SiR″₂, AsR″₂ and CR″₂;
M is a complexing metal;
X is selected from the group consisting of H, F, Br, Cl, I, OTf, dba (dibenzylidene acetone), OC(═O)CF₃ and OAc;
L is selected from the group consisting of PR″₂, NR″₂, OR″, SR″, SiR″₃, AsR″₃, alkene, alkyne, aryl and heteroaryl, each of said alkene, alkyne, aryl and heteroaryl being optionally substituted, for example with one or more halogens and/or with one or more R groups as defined herein;
each R is independently alkyl, cycloalkyl, heterocyclyl, heterocycloalkyl, aryl or heteroaryl;
D is absent or is ═S or ═O or —Z-linker-Z—, where each Z independently is O or NH or N-alkyl and linker is an alkyl chain of 2-5 carbon atoms in length;
each R″ is independently H, alkyl, cycloalkyl, heterocycloalkyl, aryl or heteroaryl, each other than H being optionally substituted, or R″₂ is —Z-linker-Z— as defined above; and
m is 0 or 1 or 2; wherein:
   if m is 0, n is 1, n′ and n″ are 0 and -- is absent; and
   if m is 1 or 2, n is 1 or 2 and n′ and n″ are integers such that the coordination sphere of M is filled, and D is absent.

25. The compound of statement 24 wherein m is 1 or 2 and M is bonded to a ring atom of Ar³ ortho to E.

26. The compound of statement 24 or statement 25 wherein E is O or S or NR″. E may be absent or O. It may be absent. It may be O.

27. The compound of any one of statements 24 to 26 wherein n is 1 or 2 and M is selected from the group consisting of Pd, Ni, Pt, Cu, Fe, Co, Au, Ag, Rh, Ir, Ru, Os or Mn.

28. The compound of statement 27 wherein M is Pd, Ni or Cu.

29. The compound of statement 28 wherein M is Pd.

30. The compound of any one of statements 24 to 29 wherein m is 1 or 2 and X is Cl or OAc.

31. The compound of statement 24 wherein m=0.

32. The compound of statement 24 wherein m=1.

33. The compound of statement 24 wherein m=2 and n″ is 0.

34. The compound of any one of statements 24 to 30, 32 or 33 wherein n=m.

35. The compound of any one of statements 24 to 34 wherein each R is independently selected from the group consisting of alkyl, cycloalkyl, aryl and heteroaryl.

36. The compound of statement 35 wherein each R is alkyl or each is aryl, or one is alkyl and the other is aryl.

37. The compound of any one of statements 24 to 30 or 32 to 35 wherein n″ is 0, M is Pd and n is 2.

38. The compound of statement 24 wherein Ar¹ is phenyl, Ar² is 2,4,6-trimethylphenyl or 2,4,6-triisopropylphenyl, Ar³ is phenyl, E is O, R attached to P is each alkyl or each is aryl, or one is alkyl and the other is aryl, and n is 1 and either m, n′ and n″ are all 0 or m is 1, n′ is 1 and n″ is 0 and M is Pd.

39. The compound of statement 24 wherein if E is absent, n is 1 and m, n′ and n″ are all 0.

In one aspect of the invention, there is provided a compound of structure I:

Ar¹—Ar²—Ar³-E-P(=D)R₂

wherein:
Ar¹, Ar² and Ar³ are aromatic groups wherein:
   Ar¹ and Ar³ are in a 1,3 relationship on Ar²;
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent, or is O or S, 2 if Y is B or N and 3 if Y is Si,
   Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms;
E is absent or is selected from the group consisting of O, S, NR″, SiR″₂, AsR″₂ and CR″₂;
each R is independently alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy;
D is absent or is ═S or ═O or —Z-linker-Z—, where each Z independently is O or NH or N-alkyl and linker is an alkyl chain of 2-5 carbon atoms in length; and
each R″ is independently H, alkyl, cycloalkyl, heterocycloalkyl, aryl or heteroaryl, each other than H being optionally substituted, or R″₂ is —Z-linker-Z— as defined above.

In one option of this aspect, and Ar² is a phenyl ring substituted in the 1 position by Ar¹ and in the 3 position by Ar³ and also substituted in the 2, 4 and 6 positions by alkyl groups such as methyl groups.

In a particular example of this aspect, M is Pd and Ar² is a phenyl ring substituted in the 1 position by Ar¹ and in the 3 position by Ar³ and also substituted in the 2, 4 and 6 positions by alkyl groups such as methyl groups.

In another particular example of this aspect, M is Pd, Ar² is a phenyl ring substituted in the 1 position by Ar¹ and in the 3 position by Ar³ and also substituted in the 2, 4 and 6 positions by alkyl groups such as methyl groups and Ar¹ and Ar³ each comprise phenyl rings bonded to Ar², and each, independently, is optionally fused with another aromatic ring, optionally a heteroaromatic ring.

In this aspect, Ar¹ and Ar³ may each comprise phenyl rings bonded to Ar², and each, independently, optionally fused with another aromatic ring, optionally a heteroaromatic ring. In this aspect, and in the particular examples thereof, D and E may each, independently, be absent or may be O. D may be absent. In some options, D and E are both absent. In other options both are O. In this aspect, and in the particular examples thereof, E-P(=D)R₂ may be in a 1,2- or a 1,3-relationship with Ar² on Ar³.

In another aspect of the invention there is provided a compound of structure I:

(Ar¹—Ar²—Ar³-E-PR₂—-)MX_{n′}L_{n″}

wherein:
Ar¹, Ar² and Ar³ are aromatic groups wherein:
   Ar¹ and Ar³ are in a 1,3 relationship on Ar²,
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent, or is O or S, 2 if Y is B or N and 3 if Y is Si,
   Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms;
E is absent or is selected from the group consisting of O, S, NR″, SiR″₂, AsR″₂ and CR″₂,
M is a complexing metal;
X is selected from the group consisting of H, F, Br, Cl, I, OTf, dba (dibenzylidene acetone), OC(═O)CF₃ and OAc;
L is absent or is selected from the group consisting of PR″₂, NR″₂, OR″, SR″, SiR″₃, AsR″₃, alkene, alkyne, aryl and heteroaryl, each of said alkene, alkyne, aryl and heteroaryl being optionally substituted, for example with one or more halogens and/or with one or more R groups as defined herein, wherein if L is absent, n is not 1;
each R is independently alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy;
D is absent or is ═S or ═O or —Z-linker-Z—, where each Z independently is O or NH or N-alkyl and linker is an alkyl chain of 2-5 carbon atoms in length;
each R″ is independently H, alkyl, cycloalkyl, heterocycloalkyl, aryl or heteroaryl, each other than H being optionally substituted, or R″₂ is —Z-linker-Z— as defined above; and
n′ and n″ are integers such that the coordination sphere of M is filled (e.g. n′=n″=1).

In one option of this aspect, and Ar² is a phenyl ring substituted in the 1 position by Ar¹ and in the 3 position by Ar³ and also substituted in the 2, 4 and 6 positions by alkyl groups such as methyl groups.

In a particular example of this aspect, M is Pd and Ar² is a phenyl ring substituted in the 1 position by Ar¹ and in the 3 position by Ar³ and also substituted in the 2, 4 and 6 positions by alkyl groups such as methyl groups.

In another particular example of this aspect, M is Pd, Ar² is a phenyl ring substituted in the 1 position by Ar¹ and in the 3 position by Ar³ and also substituted in the 2, 4 and 6 positions by alkyl groups such as methyl groups and Ar¹ and Ar³ each comprise phenyl rings bonded to Ar², and each, independently, is optionally fused with another aromatic ring, optionally a heteroaromatic ring.

In this aspect, Ar¹ and Ar³ may each comprise phenyl rings bonded to Ar², and each, independently, optionally fused with another aromatic ring, optionally a heteroaromatic ring. In this aspect, and in the particular examples thereof, D and E may each, independently, be absent or may be O. In some options, D and E are both absent. In other options both are O. In this aspect, and in the particular examples thereof, E-P(=D)R₂ may be in a 1,2- or a 1,3-relationship with Ar² on Ar³. In this aspect, and in the particular examples thereof, n′ and n″ may be 1.

In yet another aspect of the invention there is provided a compound of structure I:

(Ar¹—Ar²—Ar³-E-PR₂—-)₂M₂X_{n′}

wherein:
Ar¹, Ar² and Ar³ are aromatic groups wherein:
   Ar¹ and Ar³ are in a 1,3 relationship on Ar²,
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent, or is O or S, 2 if Y is B or N and 3 if Y is Si,
   Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms;
E is absent or is selected from the group consisting of O, S, NR″, SiR″₂, AsR″₂ and CR″₂;
M is a complexing metal;
X is selected from the group consisting of H, F, Br, Cl, I, OTf, dba (dibenzylidene acetone), OC(═O)CF₃ and OAc;
each R is independently alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy;
D is absent or is ═S or ═O or —Z-linker-Z—, where each Z independently is O or NH or N-alkyl and linker is an alkyl chain of 2-5 carbon atoms in length;
each R″ is independently H, alkyl, cycloalkyl, heterocycloalkyl, aryl or heteroaryl, each other than H being optionally substituted, or R″₂ is —Z-linker-Z— as defined above; and
and n′ is an integer such that the coordination sphere of M is filled (e.g. n′ is 2).

In one option of this aspect, and Ar² is a phenyl ring substituted in the 1 position by Ar¹ and in the 3 position by Ar³ and also substituted in the 2, 4 and 6 positions by alkyl groups such as methyl groups.

In a particular example of this aspect, M is Pd and Ar² is a phenyl ring substituted in the 1 position by Ar¹ and in the 3 position by Ar³ and also substituted in the 2, 4 and 6 positions by alkyl groups such as methyl groups.

In another particular example of this aspect, M is Pd, Ar² is a phenyl ring substituted in the 1 position by Ar¹ and in the 3 position by Ar³ and also substituted in the 2, 4 and 6 positions by alkyl groups such as methyl groups and Ar¹ and Ar³ each comprise phenyl rings bonded to Ar², and each, independently, is optionally fused with another aromatic ring, optionally a heteroaromatic ring.

In this aspect, Ar¹ and Ar³ may each comprise phenyl rings bonded to Ar², and each, independently, optionally fused with another aromatic ring, optionally a heteroaromatic ring. In this aspect, and in the particular examples thereof, D and E may each, independently, be absent or may be O. In some options, D and E are both absent. In other options both are O. In this aspect, and in the particular examples thereof, E-P(=D)R₂ may be in a 1,2- or a 1,3-relationship with Ar² on Ar³. In this aspect, and in the particular examples thereof, n′ may be 2.

40. A process for making a coupled compound comprising reacting a compound of structure R^{a}X in which R^{a} is alkyl, alkenyl, alkynyl, aryl or heteroaryl and X is a leaving group such as halide or triflate, with a compound V comprising an element M bonded directly to an alkyl, alkenyl, alkynyl, aryl or heteroaryl group, in which M is an element in one of groups 1, 2, 11, 12, 13 or 14, in the presence of either:
a compound of structure I as defined in statement 24 in which m=1 or 2 and M=Pd, or
a compound of structure I as defined in statement 24 in which m=0 and a palladium salt or complex capable of reacting with the compound of structure I to form a compound of structure I in which m=1 or 2 and M=Pd.

41. The process of statement 40 wherein either R^{a} or compound V or both is substituted with one or more substituents.

42. The process of statement 41 wherein each of the one or more substituents is, independently, alkyl, aryl, heteroaryl, halogen, OR^{b}, NR^{b}R^{c}, BR^{b}R^{c}, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl.

43. The process of statement 42 wherein compound V is a boronic acid.

44. A process for making a olefinic compound comprising reacting a compound of structure R^{a}X in which R^{a} is alkyl, alkenyl, alkynyl, aryl or heteroaryl and X is a leaving group such as halide or triflate with an olefin in the presence of either:
a compound of structure I as defined in statement 24 in which m=1 or 2 and M=Pd, or
a compound of structure I as defined in statement 24 in which m=0 and a palladium salt or complex capable of reacting with the compound of structure I to form a compound of structure I in which m=1 or 2 and M=Pd.

45. The process of statement 44 wherein either R^{a} or the olefin or both is substituted with one or more substituents.

46. The process of statement 45 wherein each of the one or more substituents is, independently, alkyl, aryl, heteroaryl, halogen, OR^{b′}, NR^{b}R″, BR^{b}R″, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl group.

47. A process for making an alkynyl compound comprising reacting a compound of structure R^{a}X in which R^{a} is alkyl, alkenyl, alkynyl, aryl or heteroaryl and X is a leaving group such as halide or triflate with a terminal alkyne in the presence of either:
a compound of structure I as defined in statement 24 in which m=1 or 2 and M=Pd, or
a compound of structure I as defined in statement 24 in which m=0 and a palladium salt capable of reacting with the compound of structure I to form a compound of structure I in which m=1 or 2 and M=Pd.

48. The process of statement 47 wherein R^{a} is substituted with one or more substituents.

49. The process of statement 48 wherein each of the one or more substituents is, independently, alkyl, aryl, heteroaryl, halogen, OR^{b}, NR^{b}R^{c}, BR^{b}R^{c}, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl.

50. A process for making an amine compound comprising reacting a compound of structure R^{a}X in which R^{a} is alkyl, alkenyl, alkynyl, aryl or heteroaryl and X is a leaving group such as halide or triflate with a primary or secondary amine in the presence of either:
a compound of structure I as defined in statement 24 in which m= or 2 and M=Pd, or
a compound of structure I as defined in statement 24 in which m=0 and a palladium salt capable of reacting with the compound of structure I to form a compound of structure I in which m=1 or 2 and M=Pd.

51. The process of statement 50 wherein R^{a} is substituted with one or more substituents.

52. The process of statement 51 wherein each of the one or more substituents is, independently, alkyl, aryl, heteroaryl, halogen, OR^{b}, NR^{b}R^{c}, BR^{b}R^{c}, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl.

53. The process of any one of statements 50 to 52 wherein the amine is of the form R^{e}NH₂ or R^{e}R^{f}NH in which R^{e} and R^{f} are, independently, alkyl, alkenyl, heteroaryl or aryl groups.

54. A process for making an aryl carbonyl compound, e.g. a ketone or aldehyde or acid or ester, comprising reacting a compound of structure R^{a}X in which R^{a} is alkyl, alkenyl, alkynyl, aryl or heteroaryl and X is a leaving group such as halide or triflate with carbon monoxide and a nucleophile M′-Nu, where Nu=OH, NR₂, alkyl, aryl, SR, or OR^{a} where each R^{a} is as described above, and M′ is H or a Group 1 or Group 2 (alkyl or alkaline earth) metal ion or is M″R^{b}, where M is Cu, Ag, Zn, AlR, GaR^{b}, TlR^{b}, CR^{b}₂, SiR^{b}₂, SnR^{b}₂, PbR^{b}₂, NR^{b} or AsR^{b}, where R^{b} is alkyl, alkenyl, alkynyl, aryl or heteroaryl, in the presence of either:
a compound of structure I as defined in statement 24 in which m=1 or 2 and M=Pd, or
a compound of structure I as defined in statement 24 in which m=0 and a palladium salt capable of reacting with the compound of structure I to form a compound of structure I in which m=1 or 2 and M=Pd.

55. The process of statement 54 wherein R^{a} is substituted with one or more substituents.

56. The process of statement 55 wherein each of the one or more substituents is, independently, alkyl, aryl, heteroaryl, halogen, OR^{c}, NR^{c}R^{d}, BR^{c}R^{d}, SiR^{c}₃ or SiR^{c}R^{d}₂ or SiR^{c}R^{d}R^{e}, in which R^{e}, R^{d} and R^{e} are each independently H, alkyl, alkenyl, alkynyl, OH, amine, halogen, alkoxyl or aryl.

57. A process for making a nitrile compound comprising reacting a compound of structure R^{a}X in which R is alkyl, alkenyl, alkynyl, aryl or heteroaryl and X is a leaving group such as halide or triflate with a cyanide in the presence of either:
a compound of structure I as defined in statement 24 in which m=1 or 2 and M=Pd, or
a compound of structure I as defined in statement 24 in which m=0 and a palladium salt capable of reacting with the compound of structure I to form a compound of structure I in which m=1 or 2 and M=Pd.

58. The process of statement 57 wherein R is substituted with one or more substituents.

59. The process of statement 58 wherein each of the one or more substituents is, independently, alkyl, aryl, heteroaryl, halogen, OR^{e}, NR^{c}R^{d}, BR^{c}R^{d}, SiR^{c}₃ or SiR^{e}R^{d}₂ or SiR^{c}R^{d}R^{e}, in which R^{c}, R^{d} and R^{e} are each independently H, alkyl, alkenyl, alkynyl, OH, amine, halogen, alkoxyl or aryl.

60. The process of any one of statements 57 to 59 wherein the cyanide is a Group I metal cyanide or HCN.

61. A process for making a phosphorus compound comprising reacting a compound of structure R^{a}X in which R^{a} is alkyl, alkenyl, alkynyl, aryl or heteroaryl and X is a leaving group such as halide or triflate with a phosphorus reagent in the presence of either:
a compound of structure I as defined in statement 24 in which m=1 or 2 and M=Pd, or
a compound of structure I as defined in statement 24 in which m=0 and a palladium salt capable of reacting with the compound of structure I to form a compound of structure I in which m=1 or 2 and M=Pd.

62. The process of statement 61 wherein R^{a} is substituted with one or more substituents.

63. The process of statement 62 wherein each of the one or more substituents is, independently, alkyl, aryl, heteroaryl, halogen, OR^{e}, NR^{c}R^{d}, BR^{c}R^{d}, SiR^{c}₃ or SiR^{e}R^{d}₂ or SiR^{c}R^{d}R^{e}, in which R^{c}, R^{d} and R^{e} are each independently H, alkyl, alkenyl, alkynyl, OH, alkoxyl or aryl.

64. The process of any one of statements 61 to 63 wherein the phosphorus reagent is X—PR^{b}₂ or X₂—PR^{b} where X═H or halide, or X₂ is ═O or ═S or the phosphorus reagent is a protected phosphine such as a borane adduct.

65. A process for making an ether comprising reacting a compound of structure R^{a}X in which R^{a} is alkyl, alkenyl, alkynyl, aryl or heteroaryl and X is a leaving group such as halide or triflate with an alcohol in the presence of either:
a compound of structure I as defined in statement 24 in which m=1 or 2 and M=Pd, or
a compound of structure I as defined in statement 24 in which m=0 and a palladium salt capable of reacting with the compound of structure I to form a compound of structure I in which m=1 or 2 and M=Pd.

66. The process of statement 65 wherein R^{a} is substituted with one or more substituents.

67. The process of statement 66 wherein each of the one or more substituents is, independently, alkyl, aryl, heteroaryl, halogen, OR^{e}, NR^{c}R^{d}, BR^{c}R^{d}, SiR^{c}₃ or SiR^{c}R^{d}₂ or SiR^{c}R^{d}R^{e}, in which R^{c}, R^{d} and R^{e} are each independently H, alkyl, alkenyl, alkynyl, OH, amine, halogen, alkoxyl or aryl.

In an embodiment there is provided process for a making a coupled compound comprising reacting an aryl halide with an alkyl or aryl boronic acid in the presence of a compound of structure Ar¹—Ar²—Ar³—PR₂, in the presence of a metal salt, or complex, optionally a palladium salt or complex, wherein:
Ar¹, Ar² and Ar³ are aromatic groups wherein:
   Ar¹ and Ar³ are in a 1,3 relationship on Ar²,
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent, or is O or S, 2 if Y is B or N and 3 if Y is Si,
   Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms; and
each R is independently alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy (e.g both Rs are Cy).

In another embodiment there is provided a process for a making a coupled compound comprising reacting an aryl halide with an alkyl or aryl boronic acid in the presence of a compound of structure (Ar¹—Ar²—Ar³—O—PR₂—-)₂M₂X₂

wherein:
Ar¹, Ar² and Ar³ are aromatic groups wherein:
   Ar¹ and Ar³ are in a 1,3 relationship on Ar²,
   each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent, or is O or S, 2 if Y is B or N and 3 if Y is Si,
   Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms;
M is a complexing metal, optionally Pd;
X is selected from the group consisting of H, F, Br, Cl, I, OTf, dba (dibenzylidene acetone), OC(═O)CF₃ and OAc; and
each R is independently alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy (e.g. both Rs are alkyl, such as iPr).

68. Use of a compound of structure I as defined in statement 24 in which m=1 or 2 and M=Pd as a catalyst.

69. Use according to statement 68, being for catalysis of a reaction selected from the group consisting of Suzuki-Miyaura, Kumada, Stille, Negishi and Hiyama coupling reactions, Heck and Sonogashira reactions, Buchwald-Hartwig amination, Heck carbonylation, alkoxylation, cyanation, phosphination and metal mediated coupling reactions. The use may also be for catalysis of a fluorination of an aryl compound bearing a leaving group, for example a haloaryl compound (e.g. a bromoaromatic compound), to the corresponding fluoroaromatic compound.

### BRIEF DESCRIPTION OF DRAWINGS

The invention is described with reference to the following figures:

FIG. 1 shows a structural description of the ligands of the present invention, as well as representative catalyst precursor examples. In the present specification (for example in FIG. 1), where reference is made to “A*STAR” compounds, ligands, catalysts etc., those refer to the compounds, ligands, catalysts etc. of the present invention unless the context indicates otherwise.

FIG. 2 shows representative cross-coupling transformations using the catalysts of the present invention.

FIG. 3 shows synthetic routes to *Phinite ligands and the Pd*Phinite catalysts, i.e. compounds of structure 1 in which E is not absent.

FIG. 4 shows a specific synthesis of *Phinite catalysts according to the invention, i.e. where E is not absent. It is noted that for the *Phinite series shown in FIG. 4, its use in catalysis may involve in situ reaction of ligand 7 of FIG. 3 with a metal species or may involve use of precatalyst 8 directly.

FIG. 5 shows a synthetic scheme for catalyst precursors (i.e. compounds I in which m=0) of the invention. These represent the *Phine series in which E is absent. It is noted that for *Phine series shown in FIG. 5, its use in catalysis commonly involves in situ reaction of ligand 7 with an appropriate metal species.

FIG. 6 shows different routes to the terphenyl nucleus used in synthesising the compounds of the present invention. In FIG. 6, Route 1 proceeds via Suzuki-type coupling; Route 2 proceeds via a Grignard-electrophile reaction and Route 3 proceeds via benzyne.

FIG. 7 shows a one-pot process from compound 4 to *Phine (Compound 7); i.e. from a biphenyl to a phosphorus-substituted terphenyl.

FIG. 8 shows a sample reaction for producing a catalyst (Compound I in which m=1) from the corresponding ligand precursor (Compound I in which m=0) by reaction with a suitable metal species.

FIG. 9 shows a specific catalyst according to the invention together with its corresponding ligand precursor.

FIGS. 10a to 10c show the nomenclature of compounds used or synthesised in the Examples.

### DESCRIPTION OF EMBODIMENTS

For convenience, certain terms employed in the specification, examples, and appended claims are collected here and where appropriate, definitions are provided.

The articles “a” and “an” are used herein to refer to one or to more than one (i.e. to at least one) of the grammatical object of the article. By way of example, “an element means one element or more than one element.

The term “nucleophile” is recognized in the art, and as used herein means a chemical moiety having a reactive pair of electrons.

The term “electrophile” is art-recognized and refers to chemical moieties which can accept a pair of electrons from a nucleophile as defined herein. Electrophilic moieties useful in the method of the present invention include halides and sulfonates.

The terms “electrophilic atom,” “electrophilic center” and “reactive center” as used herein refer to an atom, commonly of a substrate aryl moiety, which is, or may be, attacked by, and forms a new bond with an incoming nucleophile. In most (but not all) cases, this will also be the aryl ring atom from which the leaving group departs.

The term “leaving group” refers to a molecular fragment that departs with a pair of electrons in heterolytic bond cleavage. Leaving groups may be anions or neutral molecules. Exemplary common leaving groups are (but are not limited to) halides such as Cl, Br, and I, and sulfonate esters, such as tosylate (TsO). Common neutral molecule leaving groups are water and ammonia.

The term “electron-withdrawing group” is recognized in the art, and denotes a substituent having a tendency to attract valence electrons from neighboring atoms (i.e. the substituent is electronegative with respect to neighboring atoms). The term “electron-donating group” denotes a substituent having a tendency to donate valence electrons to neighboring atoms (i.e. the substituent is electropositive with respect to neighboring atoms). A quantification of the level of electron-withdrawing capability is given by the Hammett sigma (a) constant. This well known constant is described in many references, such as in Smith, M. B.; March, J. March's Advanced Organic Chemistry, 5^{th} Edition; John Wiley & Sons: New York, 2001; pp 368-375. The Hammett constant values are generally negative for electron donating groups (σₚ=−0.57 for NH₂) and positive for electron withdrawing groups (σₚ=0.81 for a nitro group), σₚ indicating para substitution. Exemplary electron-withdrawing groups include nitro, ketone, ester, sulfonyl, trifluoromethyl, cyano, chloride, and the like. Exemplary electron-donating groups include amino, methoxy, and the like.

The term “activating group” (A) refers to a substituent on any carbon atom such that the presence of substituent (A) augments the polarization of the C-A bond to a greater extent than a C—H or C—C single bond in the same position (direct replacement of A with C or H). In other words, the bond polarity (measured by the dipole moment, μ) μ(C-A)>μ(C—C), or μ(C-A)>μ(C—H).

The term “ligand” is an atom, ion, or molecule that donates or shares one or more of its electrons through a covalent bond with a central atom or ion. It is a complexing group in coordination chemistry that may stabilize the central atom and may at least partially determine its reactivity. The term “liganding” or “ligating” refers to the act of the ligand coordinating with or complexing with a transition metal. In this specification the term word “liganding species” includes both charged and neutral ligands.

The term “complexing” refers to the act of a ligand coordinating with a transition metal, or describing the transition metal's capacity to accept coordination from a ligand to form a transition metal complex.

The term “catalytic amount” or “cat.” is recognized in the art and means a sub-stoichiometric amount of reagent relative to a reactant. As used herein, a catalytic amount means from 0.000001 to 90 mole percent reagent relative to a reactant, more preferably from 0.001 to 50 mole percent, still more preferably from 0.01 to 5 mole percent reagent to reactant. It may denote a reagent to reactant mole percent ratio of about 0.001, 0.005, 0.01, 0.02, 0.05, 0.1, 0.2, 0.3, 0.4, 0.5, 0.6, 0.7, 0.8, 0.9, 1, 1.5, 2, 2.5, 3, 3.5, 4, 4.5 or 5.

The term “heteroatom” is art-recognized and refers to an atom of any element other than carbon or hydrogen. Illustrative heteroatoms include boron, nitrogen, oxygen, phosphorus, sulfur and selenium.

The terms “olefin” and “alkenyl” as used herein, means a straight or branched chain hydrocarbon containing from 2 to 10 carbons and containing at least one carbon-carbon double bond. This may be formed by the removal of two hydrogens from adjacent carbon atoms of an alkane or alkyl group. Representative examples of olefin or alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein.

Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “arylalkoxy” and “heteroalkoxy” as used herein, means an aryl group or heteroaryl group, as defined herein, appended to the parent molecular moiety through an alkoxy group, as defined herein. Representative examples of arylalkoxy include, but are not limited to, 2-chlorophenylmethoxy, 3-trifluoromethylethoxy, and 2,3-methylmethoxy.

The term “arylalkyl” as used herein, means an aryl group, as defined herein, appended to the parent molecular moiety through an alkyl group, as defined herein. Representative arylalkyl groups include phenylmethyl (α-tolyl), 2-phenylethyl and naphthylmethyl.

The term “alkoxylalkyl” as used herein means an alkoxy group, as defined herein, appended to a parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkyl” means a straight or branched chain or cyclic hydrocarbon. It may contain from 1 to 10 carbon atoms or 1 to 5, or 2 to 10, or 5 to 10, or 3 to 8 carbon atoms (wherein if the alkyl is branched or cyclic, it contains at least 3 carbon atoms, commonly from 3 to 10 carbon atoms). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, cyclopentyl and cyclohexyl.

The term “alkyl carbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” and “arylcarbonyloxy” as used herein, means an alkylcarbonyl or arylcarbonyl group, as defined herein, appended to the parent molecular moiety through an oxygen atom. Representative examples of alkylcarbonyloxy include, but are not limited to, acetyloxy (or more commonly acetoxy), ethylcarbonyloxy, and tert-butylcarbonyloxy. Representative examples of arylcarbonyloxy include, but are not limited to phenylcarbonyloxy.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The terms “alkyne” and “alkynyl” as used herein, means a straight or branched chain hydrocarbon group containing from 2 to 10 carbon atoms and containing at least one carbon-carbon triple bond. Representative examples of alkyne or alkynyl include, but are not limited, to acetylenyl, i-propynyl, 2-propynyl, 3-butynyl, 2-pentynyl, and i-butynyl. It should be noted that an alkyne, or an alkynyl group, may contain a double bond as well as the triple bond. Therefore, for example, pent-2-ene-4-yne may be classed as both an alkyne and an alkene.

The term “amido” as used herein, means —NHC(═O)—, wherein the amido group is bound to the parent molecular moiety through the nitrogen. Examples of amido include alkyl amido such as H₃CC(═O)N(H)— and CH₃CH₂C(═O)N(H)—. The term “amido” may also refer to an amine ligand (—NR′₂) coordinated to a metal, where R′ is independently H, halogen, alkyl, cycloalkyl, heterocyclyl, cycloheteroaryl, aryl or heteroaryl. Examples of a metal coordinated amido group would be the —N(CH₃)₂ groups of tetrakis(dimethylamido)-titanium(IV), Ti(N(CH₃)₂)₄, or in tetrakis(dimethylamido)zirconium(IV), Zr(N(CH₃)₂)₄.

The term “amino” as used herein, refers to radicals of both unsubstituted and substituted amines appended to the parent molecular moiety through a nitrogen atom. The two groups attached to the nitrogen atom are each independently hydrogen, alkyl, alkylcarbonyl, alkyl sulfonyl, arylcarbonyl, or formyl. Representative examples include, but are not limited to dimethylamino, methylamino, acetylamino, and acetylmethylamino. It should be noted that the term “amino” may also refer to an amino group with two hydrogen substituents: H₂N—.

The term “aromatic” refers to a planar or polycyclic structure characterized by a cyclically conjugated molecular moiety containing 4n+2 electrons, wherein n is the absolute value of an integer—n may be for example 1, 2, 3, 4 or 5. Aromatic molecules containing fused, or joined, rings also are referred to as bicylic aromatic rings, e.g. naphthyl. For example, bicyclic aromatic rings containing heteroatoms in a hydrocarbon ring structure are referred to as bicyclic heteroaryl rings.

The term “aryl,” as used herein, means a phenyl group or a naphthyl group. The aryl groups of the present invention can be optionally substituted with 1, 2, 3, 4 or 5 substituents independently selected from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, halo alkyl, hydroxyl, hydroxyalkyl, mercapto, nitro, phosphinyl, silyl and silyloxy.

The term “arylene,” is art-recognized, and as used herein, pertains to a bidentate moiety obtained conceptually by removing two hydrogen atoms of an aryl ring, as defined above.

The term “arylalkyl” or “aralkyl” as used herein, means an aryl group, as defined herein, appended to the parent molecular moiety through an alkyl group, as defined herein. Representative examples of aryl alkyl include, but are not limited to, benzyl, 2-phenylethyl, 3-phenylpropyl, and 2-naphth-2-ylethyl.

The term “arylalkoxy” or “arylalkyloxy” as used herein, means an arylalkyl group, as defined herein, appended to the parent molecular moiety through an oxygen atom. The term “heteroarylalkoxy” as used herein, means a heteroarylalkyl group, as defined herein, appended to the parent molecular moiety through an oxygen atom.

The term “arylalkylthio” as used herein, means an aryl alkyl group, as defined herein, appended to the parent molecular moiety through a sulfur atom. The term “heteroarylalkylthio” as used herein, means a heteroarylalkyl group, as defined herein, appended to the parent molecular moiety through a sulfur atom.

The term “arylalkenyl” as used herein, means an aryl group, as defined herein, appended to the parent molecular moiety through an alkenyl group. A representative example is phenylethylenyl (phenylethenyl).

The term “arylalkynyl” as used herein, means an aryl group, as defined herein, appended to the parent molecular moiety through an alkynyl group. A representative example is phenylethynyl.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “arylcarbonylalkyl” as used herein, means an arylcarbonyl group, as defined herein, bound to the parent molecule through an alkyl group, as defined herein.

The term “arylcarbonylalkoxy” as used herein, means an arylcarbonylalkyl group, as defined herein, bound to the parent molecule through an oxygen atom.

The term “aryloxy” as used herein, means an aryl group, as defined herein, appended to the parent molecular moiety through an oxygen atom. The term “heteroaryloxy” as used herein, means a heteroaryl group, as defined herein, appended to the parent molecular moiety through an oxygen atom.

The term “biaryl” or “biphenyl” refers to two covalently bonded aromatic ring systems. The numbers around the peripheries of the ring systems are the positional numbering systems used herein. Likewise, the capital letters contained within the individual rings of the ring systems are the ring descriptors used herein.

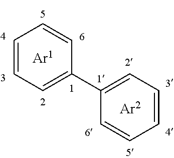 [SMILES: c1ccc(-c2ccccc2)cc1]

The term “teraryl” or “triaryl” or “terphenyl” refers to three covalently bonded aromatic ring systems in a 1, 3 configuration (e.g. a meta arrangement within a 6-membered aromatic ring system such as ring B, as demonstrated by the 3-ringed system shown below). The numbers around the peripheries of the ring systems are the positional numbering systems used herein. Likewise, the capital letters contained within the individual rings of the ring systems are the ring descriptors used herein.

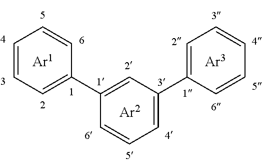 [SMILES: c1ccc(-c2cccc(-c3ccccc3)c2)cc1]

Any position on the biaryl or teraryl may be substituted with a substituent independently selected from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, halo alkyl, hydroxyl, hydroxyalkyl, mercapto, nitro, phosphinyl, silyl and silyloxy.

The term “phenyl-heteroaryl” and “heteroaryl-heteroaryl” refer to ring systems similar to those shown above for biaryl or teraryl, wherein one of the phenyl rings is replaced with an heteroaryl ring, as defined below. Any position on the phenyl-heteroaryl or heteroarylheteroaryl may be substituted with a substituent independently selected from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, halo alkyl, hydroxyl, hydroxyalkyl, mercapto, nitro, phosphinyl, silyl and silyloxy.

The term “carbonyl” as used herein, means a —C(═O)— group.

The term “carboxy” as used herein, means a —CO₂H group or a —CO₂⁻ group.

The term “cycloalkyl” as used herein, means mono cyclic or multicyclic (e.g. bicyclic, tricyclic, etc.) hydrocarbons containing from 3 to 12 carbon atoms that is completely saturated or has one or more unsaturated bonds but is not an aromatic group. Examples of a cycloalkyl group include cyclopropyl, cyclobutyl, cyclopentyl, cyclopentenyl, cyclohexyl and cyclohexenyl.

The term “heterocycloalkyl” as used herein, means a cycloalkyl group, as defined herein, in which at least one ring atom has been replaced by a heteroatom, e.g. O, N, S, B, Si and P. It may refer to a cycloalkyl group which is appended to the parent molecular moiety through a heteroatom such as O, N, S; B, Si and P. This term may therefore include mono-heteroatomic cyclic alkyls (e.g. piperidine), di-heteroatomic cyclic alkyls (e.g. morpholine) as well as functionalized cycloalkyls (e.g. cyclohexylamine).

The term “cyano” as used herein, means a —CN group.

The term “formyl” as used herein, means a —C(═O)H group.

The term “halo” or “halogen” means —Cl, —Br, —I, or —F.

The term “haloalkoxy” as used herein, means at least one halogen, as defined herein, appended to the parent molecular moiety through an alkoxy group, as defined herein. Representative examples of haloalkoxy include, but are not limited to chloromethoxy, 2-fluoroethoxy, trifluoromethoxy, and pentafluoroethoxy.

The term “haloalkyl” means at least one halogen, as defined herein, appended to the parent molecular moiety through an alkyl group, as defined herein. Representative examples of halo alkyl include, but are not limited to, chloromethyl, 2-fluoroethyl, trifluoromethyl, pentafluoroethyl, and 2-chloro-3-fluoropentyl.

The term “heterocyclyl”, as used herein, includes non-aromatic ring systems, including, but not limited to, monocyclic, bicyclic and tricyclic rings, which can be completely saturated or which can contain one or more units of unsaturation (for the avoidance of doubt, the degree of unsaturation does not result in an aromatic ring system). They may have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the invention may be substituted with 0, 1, 2, 3, 4 or 5 substituents independently selected from alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, halo alkyl, hydroxyl, hydroxyalkyl, mercapto, nitro, phosphinyl, silyl and silyloxy.

The term “heteroaryl” as used herein, include aromatic ring systems, including, but not limited to, monocyclic, bicyclic and tricyclic rings. They may have 3 to 12 (e.g. 5, 6, 7, 8, 9 or 10) atoms including at least one heteroatom, such as nitrogen, oxygen, or sulfur. For purposes of exemplification, which should not be construed as limiting the scope of this invention, heteroaryl groups may be: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the invention may be substituted with 0, 1, 2, 3, 4 or 5 substituents independently selected from alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, halo alkyl, hydroxyl, hydroxyalkyl, mercapto, nitro, phosphinyl, silyl and silyloxy.

The term “heteroarylene,” is art-recognized, and as used herein, pertains to a bidentate moiety obtained conceptually by removing two hydrogen atoms from adjacent ring atoms of a heteroaryl ring, as defined above.

The term “heteroarylalkyl” or “heteroaralkyl” as used herein, means a heteroaryl, as defined herein, appended tothe parent molecular moiety through an alkyl group, as defined herein. Representative examples of heteroarylalkyl include, but are not limited to, pyridin-3-ylmethyl and 2-(thien-2-yl)ethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “phosphinyl” as used herein includes derivatives of the H₃P— group, wherein the hydrogens are independently replaced with alkyl, adamantyl, fluoroalkyl, cycloalkyl, aryl, heteroaryl, heterocycyl, aryloxy, or heteroaryloxy groups.

The term “silyl” as used herein includes hydrocarbyl derivatives of the silyl (H₃Si—) group (i.e. (hydrocarbyl)₃Si—), wherein a hydrocarbyl groups are univalent groups formed by removing a hydrogen atom from a hydrocarbon (e.g. ethyl, phenyl). The hydrocarbyl groups can be combinations of differing groups which can be varied in order to provide a number of silyl groups, such as trimethylsilyl (TMS), tert-butyldiphenylsilyl (TBDPS), tertbutyldimethylsilyl (TBS/TBDMS), triisopropylsilyl (TIPS), and [2-(trimethylsilyl)ethoxy]methyl (SEM).

The term “silyloxy” as used herein means a silyl group, as defined herein, which is appended to the parent molecule through an oxygen atom.

The definition of each expression (e.g. alkyl, m, n, and the like) when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methane sulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List ofAbbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound (e.g. which does not spontaneously undergo transformation such as by rearrangement, cyclization, elimination, or other reaction).

The term “substituted” is also contemplated to include all permissible substituents of organic compounds. In a broad aspect, the permissible substituents include acyclic and cyclic, branched and unbranched, carbocyclic and heterocyclic, aromatic and non-aromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2nd Edition; Wiley: New York, 1991). Protected forms of the inventive compounds are included within the scope of this invention.

A “polar solvent” means a solvent which has a dielectric constant (Cᵣ), also known as relative permittivity, of 2.9 or greater, such as DMF, THF, ethylene glycol dimethyl ether, DME, DMSO, acetone, acetonitrile, methanol, ethanol, isopropanol, n-propanol, t-butanol or 2-methoxyethyl ether. Preferred polar solvents are DMF, DME, NMP, and acetonitrile. A “non-polar” solvent is a solvent which is not a polar solvent, i.e. has a dielectric constant of less than 2.9.

An “aprotic solvent” means a non-nucleophilic solvent having a boiling point range above ambient temperature, preferably from about 20° C. to about 190° C., more preferably from about 80° C. to about 160° C., most preferably from about 80° C. to 150° C., at atmospheric pressure. Examples of such solvents are acetonitrile, toluene, DMF, diglyme, THF or DMSO.

A “polar, aprotic solvent” means a polar solvent as defined above which has no available hydrogens to exchange with the compounds of this invention during reaction, for example DMF, acetonitrile, diglyme, DMSO, or THF.

A “hydroxylic solvent” means a solvent that comprises a hydroxyl moiety; for example, water, methanol, ethanol, tert-butanol, and ethylene glycol are hydroxylic solvents.

For purposes of this invention, the chemical elements are identified in accordance with the Periodic Table of the Elements, CAS version, Handbook of Chemistry and Physics, 67^{th} Edition, 1986-87, inside cover.

The following abbreviations may be used in the present specification:

cat.=catalytic
Me=methyl
iPr=isopropyl
tBu=tert-butyl
Cy=cyclohexyl
Ph=phenyl
Mes=mesityl, or 2,4,6-trimethylphenyl
TIP=TIPP=2,4,6-tri-isopropylphenyl
DIBAL=Diisobutylaluminum hydride
OTf=triflate, or trifluoromethanesulfonate
OTs=tosylate, 4-toluenesulfonate
OMs=mesylate, or methanesulfonate
OAc=acetate
dba=dibenzylideneacetone
PhMe=toluene
DCM=dichloromethane
Diox=1,4-dioxane
THF=tetrahydrofuran
DMF=dimethylformamide
Selectfluor=1-Chloromethyl-4-fluoro-1,4-diazoniabicyclo[2.2.2]octane bis(tetrafluoroborate)
PEPPSI-iPr=[1,3-Bis(2,6-Diisopropylphenyl)imidazol-2-ylidene](3-chloropyridyl)palladium(II) dichloride
PEPPSI-SIPr=(1,3-Bis(2,6-diisopropylphenyl)imidazolidene) (3-chloropyridyl) palladium(II) dichloride
SPhos=2-Dicyclohexylphosphino-2′,6′-dimethoxybiphenyl
XPhos=2-Dicyclohexylphosphino-2′,4′,6′-triisopropylbiphenyl
RuPhos=2-Dicyclohexylphosphino-2′,6′-diisopropoxybiphenyl

### DavePhos=2-Dicyclohexylphosphino-2′-(N,N-dimethylamino)biphenyl

TON=Turnover number

The invention relates to phosphorus-containing catalysts which contain phosphorus substituents. These are based on the meta-terphenyl ring structure shown earlier under the definition of “teraryl”. The phosphorus atom is commonly attached to one of the terminal phenyl rings of this structure, either directly or through a spacer atom (O, S, N, Si, As or C), commonly to a carbon of the meta-terphenyl structure in position 2 or 3. It will be recognised that if the spacer atom is N, Si, As or C, it will have one or more other subsituents as well. These substituents may be hydrogen, or may be alkyl, aryl or related groups. The phosphorus atom, as well as being bonded (optionally through a spacer atom) to the meta-terphenyl ring structure, has two other substituents each of which may be alkyl, aryl or related groups. The phosphorus atom is also complexed to a metal atom. The metal atom may be any suitable metal atom capable of complexing with phosphorus. It is commonly a transition metal. It may be a Group 8, 9, 10 or 11 transition metal. Suitable metals include Pd, Ni, Pt, Cu, Fe, Co, Au, Ag, Rh, Ir, Ru, Os and Mn. In many examples the metal atom is palladium.

The metal M may have an ion or other atom or group X associated with it. This may be for example H, F, Br, Cl, I, OTf, dba (dibenzylidene acetone), OC(═O)CF₃ and OAc. It will be understood that the metal M should have its coordination sphere filled. This may be accomplished in a number of ways:
It may have a ligand L liganded thereto. L may be for example a phosphine, phosphate, an amine, alkoxy, thioalkoxy, silane, arsine, alkene, alkyne or other liganding species.
It may be liganded to the meta-terphenyl ring system, so as to form a ring comprising two adjacent carbon atoms on a terminal ring of the meta-terphenyl ring system, the metal atom, the phosphorus atom and optionally the spacer atom.
The catalyst may be dimerised through the metal atom. Thus the dimer may comprise a ring comprising two non-adjacent metal atoms and two X groups, where each metal atom is coupled to a terphenyl group (optionally identical terphenyl groups) as described above.

Thus in a broad form of the invention the catalyst is a compound which comprises the (Ar¹—Ar²—Ar³-E-PR₂—-)M group, in which E may be absent or may be a spacer atom. Commonly each of Ar¹, Ar² and Ar³ is a phenyl ring, i.e. a monocyclic homoaromatic (aryl) ring and Ar¹ and Ar³ are in a meta relationship on Ar². More broadly, each Ar group should be monocyclic and each, independently, may be aromatic or heteroaromatic. Suitable heteroaromatic rings include pyridine, pyrazole, thiophene, pyrole. In particular embodiments each, independently, may be 5 membered or 6 membered. In some-embodiments they are each 6 membered aromatic or heteroaromatic rings. In many embodiments, Ar¹, Ar² and Ar³ are all monocyclic carbocyclic aromatic rings.

In the case where E is a spacer atom (e.g. O) and m is 0 and n is 1 (i.e. the uncomplexed ligand), there may be at least one unsubstituted ring carbon atom ortho to the carbon atom to which E is attached in Ar³. This allows for complexing of a metal atom such as Pd both to the P atom attached to E and to the ortho carbon atom of Ar³ so as to form a 5-membered ring. Thus if E is attached to Ar³ ortho to Ar², the carbon atom of Ar³ meta to Ar² and ortho to E may have a hydrogen atom, and if E is attached to Ar³ meta to Ar², the carbon atom of Ar³ between Ar² and E or the carbon atom para to Ar² and ortho to E may have a hydrogen atom.

The compounds of the invention may therefore be regarded as ligands based on the meta-terphenyl aromatic nucleus having phosphorus containing groups attached to a terminal ring of the nucleus, as well as metal complexes in which such a ligand is complexed to a metal atom or ion. It will be understood that other groups may also be complexed to the same metal atom or ion, e.g. counterions or other liganding species. In some instances, more than one (e.g. 2) of the meta-terphenyl based ligands may be complexed to the same metal atom or ion.

The spacer atom may be absent (in which P is directly attached to Ar³), or may be, for example O, S, N, Si or As or some other suitable atom. In the case of divalent atoms (O, S), this will represent E in structure I, however if the spacer atom has a valency greater than 2, the group E will comprise the spacer atom with suitable substituents. These may be H, alkyl, cycloalkyl, heterocyclyl, heterocycloalkyl aryl or heteroaryl. Each of these, other than H, may be substituted, for example with one or more halogens and/or alkyl groups and/or aryl groups. In some instances R″₂ may form a ring with the spacer atom, i.e. may be —Z-linker-Z— where each Z independently is O or NH or N-alkyl and linker is an alkyl chain of 2-5 carbon atoms in length.

Specific examples of the catalysts of the invention include:
(Ar¹—Ar²—Ar³-E-PR₂—-)MXL in which E is attached to position 2″ of Ar³ and M is attached to position 3″ of Ar³. In a specific example, E is O and M is Pd.
(Ar¹—Ar²—Ar³-E-PR₂—-)MXL in which E is attached to position 3″ of Ar³ and M is attached to position 2″ of Ar³. In a specific example, E is O and M is Pd.
(Ar¹—Ar²—Ar³—PR₂—-)MX_{n′}L_{n″} in which P is attached to position 2″ of Ar³. In a specific example, M is Pd.
(Ar¹—Ar²—Ar³—PR₂—-)MX_{n′}L_{n″} in which P is attached to position 3″ of Ar³. In a specific example, M is Pd.
(Ar¹—Ar²—Ar³-E-PR₂—-)₂M₂X₂ which is a dimer through the two MX pairs and in which each E is attached to position 2″ of Ar³ and each M is attached to position 3″ of Ar³. In a specific example, E is O and M is Pd.
(Ar¹—Ar²—Ar³-E-PR₂—-)₂M₂X₂ which is a dimer through the two MX pairs and in which each E is attached to position 3″ of Ar³ and each M is attached to position 2″ of Ar³. In a specific example, E is O and M is Pd.
(Ar¹—Ar²—Ar³-E-PR₂—-)₂MX₂ in which each E is attached to position 2″ of Ar³ and each M is attached to position 3″ of Ar³. In a specific example, E is O and M is Pd. In another specific example E is absent and M is Pd.
(Ar¹—Ar²—Ar³-E-PR₂—-)₂MX₂ in which each E is attached to position 3″ of Ar³ and each M is attached to position 2″ of Ar³. In a specific example, E is O and M is Pd.

In another specific example E is absent and M is Pd.

In the formula (Ar¹—Ar²—Ar³-E-PR₂—-)ₙMₘX_{n′}L_{n″}, commonly m and n are the same although in some examples (as noted above), m may be 1 and n may be 2. Thus if the catalyst is a monomeric species, n is 1 and there is commonly only one metal atom attached. In this case, it is common for X and L to also be 1 however this will depend on the nature (in particular the coordination sphere) of M. If the catalyst is a dimeric species, n is 2 and, since the dimerisation is commonly through the M-L pair, m and n′ will be 2. In this case, n″ is commonly 0. Thus common values for m, n, n′ and n″ are: 1, 1, 1, 1 respectively or 2, 2, 2, 0. However if m and n are both 1, n′ and n″ may be greater than 1 (e.g. 2, 3 or 4) if necessary to fill the coordination sphere of M. In this context, the “coordination sphere” refers to the first coordination sphere of the metal atom M. Additionally, the term “metal atom” should be taken to include ions of the metal, i.e. a metal atom having one or more electrons removed therefrom. In certain instances, the catalyst may be trimeric or larger. The skilled person will readily appreciate suitable values for m, n, n′ and n″ for such species from the above description of the dimer. Thus, suitable ranges for m, n and n′, independently, include 1, 2, 3 or 4 and for n″ include 0, 1, 2, 3 or 4.

In some embodiments the central ring Ar² has a single hydrogen atom directly attached thereto. In particular, it may be a monocyclic aryl ring having a single hydrogen attached thereto. The single hydrogen atom may be in position 5′. As Ar¹ is attached at position 1′ and Ar³ is attached at position 3′, Ar² may be substituted at positions 2′, 4′ and 6′. These substituents may be identical or they may be different. They may be alkyl groups. They may be C1 to C6 alkyl groups. They may for example be methyl, ethyl, 1-propyl, 2-propyl, 1-n-butyl, 2-n-butyl, tert-butyl, 1-n-hexyl, cyclopentyl or some other alkyl group. Each of these may be unsubstituted or may be substituted, e.g. with one or more halogens or one or more aryl groups.

In some instances D is absent. In other instances D is ═S or ═O. In yet other instances, D is —Z-linker-Z—, where each Z independently is O or NH or N-alkyl and linker is an alkyl chain of 2-5 (e.g. 2, 3, 4 or 5) carbon atoms in length

The R groups attached to P may be alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy. In many instances the R groups on P are the same. They may, independently, be bulky groups. They may for example be alkyl groups which are branched from the carbon attached to the phosphorus (e.g. i-Pr, t-Bu, 1,1-dimethylethyl), or may be cycloalkyl or heterocyclyl groups in which a ring atom is attached directly to the phosphorus (e.g. cyclohexyl, cyclopentyl, N-morpholinyl, 2-morpholinyl), or may be aromatic or heteroaromatic groups (e.g. phenyl, naphthyl, pyridyl, benzothiophenyl). In certain embodiments, the R groups are not adamantyl groups. They may not be cage groups. They may not be bicyclo- or tricyclo-aliphatic compounds. They may have no ring structures. In some embodiments the two R groups together with the phosphorus atom to which they are attached form a non-aromatic, optionally saturated, ring.

In one embodiment there is provided a compound of structure

(Ar¹—Ar²—Ar³-E-PR₂—-)MXL

wherein:
Ar¹ and Ar³ are aryl groups, and Ar² is a monocyclic aryl group,
Ar² is unsubstituted at position 5′ and is substituted at positions 2′, 4′ and 6′ by C1 to C6 alkyl groups and is substituted at position 1′ by Art and at position 3′ by Ar³;
E is absent or is O;
either E is attached to position 2″ of Ar³ and M is attached to position 3″ of Ar³ or E is attached to position 3″ of Ar³ and M is attached to position 2″ of Ar³
M is a complexing metal selected from the group consisting of Pd, Ni, Pt, Cu, Fe, Co, Au, Ag, Rh, Ir, Ru, Os or Mn
X is selected from the group consisting of Cl, OTf, dibenzylidene acetone, OC(═O)CF₃ and OAc;
L is selected from the group consisting of PR″₂, NR″₂, OR″, SR″, SiR″₃, AsR″₃, alkene, alkyne, aryl and heteroaryl,
each R is independently alkyl, aryl, heteroaryl, alkyloxy, aryloxy or heteroaryloxy; and
each R″ is independently H, alkyl, aryl or heteroaryl.

In another embodiment there is provided a compound of structure

(Ar¹—Ar²—Ar³-E-PR₂—-)₂M₂X₂

wherein:
Ar¹ and Ar³ are aryl groups, and Ar² is a monocyclic aryl group,
Ar² is unsubstituted at position 5′ and is substituted at positions 2′, 4′ and 6′ by C1 to C6 alkyl groups and is substituted at position 1‘ by Ar’ and at position 3′ by Ar³;
E is absent or is O;
either E is attached to position 2″ of Ar³ and M is attached to position 3″ of Ar³ or E is attached to position 3″ of Ar³ and M is attached to position 2″ of Ar³
M is a complexing metal selected from the group consisting of Pd, Ni, Pt, Cu, Fe, Co, Au, Ag, Rh, Ir, Ru, Os or Mn
X is selected from the group consisting of Cl, OTf, dibenzylidene acetone, OC(═O)CF₃ and OAc;
each R is independently alkyl, aryl, heteroaryl, alkyloxy, aryloxy or heteroaryloxy; and
each R″ is independently H, alkyl, aryl or heteroaryl.

The synthesis of the compounds of the invention may be accomplished by a variety of routes. If E is absent, i.e. phosphorus is directly attached to the teraryl ring structure, a 2- or 3-halosubstituted teraryl compound may be reacted with a suitable phosphorus compound in order to attach the phosphorus to the ring. The halide in this instance may be chlorine, bromine or iodine. The coupling may for example be a Cu(I) and/or Li and/or Mg mediated coupling of the teraryl halide with a halophosphorus compound (e.g. HalPR₂), or by formation of a Grignard reagent of the teraryl halide and reaction with a halophosphorus compound. In these couplings, Cu(I) may be used in catalytic quantities to perform the phosphination of the teraryl scaffold. However, Cu(I) may in some cases also be used in stoichiometric quantities. By contrast in these reactions Li and Mg are used in stoichiometric quantities or greater.

If E is present, the introduction of phosphorus may be accomplished from the corresponding -EH substituted teraryl compound, i.e. the teraryl compound in which an -EH group is present in the location on the ring system where the -EP group is to be introduced. In this case, treatment with a base of sufficient strength to abstract the H from the -EH group forms an anion which can then be reacted with a halophosphorus compound to form the desired phosphorus compound. Suitable bases include sodium hydride, potassium hydride, lithium hydride, tert-butoxide, lithium diisopropylamide etc.

Reaction of the above phosphorus compounds with a salt MₐX_{b} (where a and b are appropriate for valencies of M and X) may provide the dimer compound (Ar¹—Ar²—Ar³-E-PR₂—-)₂M₂X₂. A suitable metal salt in this instance include PdCl₂, so as to produce the dimer (Ar¹—Ar²—Ar³-E-PR₂—-)₂Pd₂Cl₂. Reaction of this dimer with a suitable complexing species (e.g. L, as described earlier) can then provide the monomeric species (Ar¹—Ar¹—Ar³-E-PR₂—-)MXL. In order to introduce ligands L which are not neutral compounds, e.g. NR₂, it is useful to react the dimer with a salt of the ligand, e.g. LiNR₂.

The compounds of the invention are useful as catalysts. They may be useful as catalysts in Suzuki-Miyaura, Kumada, Stille, Negishi and Hiyama coupling reactions, Heck and Sonogashira reactions, Buchwald-Hartwig amination, Heck carbonylation, alkoxylation, cyanation, phosphination and metal mediated coupling reactions. They may be useful in aryl-aryl coupling reactions such as Suzuki-Miyaura (or Suzuki) coupling reactions. They may be useful in aryl-vinyl coupling reactions. Many of the reactions that may be catalysed by the catalysts of the invention involve coupling unsaturated species to other species, often to other unsaturated species. The unsaturated species may be olefins, alkynes or aromatic or heteroaromatic compounds. The coupling may involve formation of carbon-carbon bonds between the two species. In some instances one of the species is a small molecule or ion such as carbon monoxide or cyanide in which case the coupling may represent the introduction of a functional group into a molecule. The catalysts may be useful in catalysing the conversion of aryl halides (e.g. chlorides, bromides) or other aryl compounds bearing a suitable leaving group, to the corresponding fluorides. The aryl halides may be substituted, e.g. with heteroaromatic groups or may be fused with heteroaryl rings. This reaction may comprise reaction of the aryl compound with a fluoride salt in the presence of a catalytic amount of the metal (e.g. palladium) complexes described herein. The fluoride salt may be for example potassium fluoride, caesium fluoride and/or silver fluoride. In addition to halides as leaving groups, other suitable groups include triflate, tosylate etc.

The compounds of the invention may be useful in the coupling of aryl halides (e.g. aryl bromides) to other species. The other species may be boronic acids or esters thereof, e.g. aryl boronic acids or esters or alkyl boronic acids or esters, or may be Grignard reagents, e.g. aryl Grignard reagents, or may be olefins, e.g. acrylic acids or derivatives (esters, amides etc.) thereof. In the case of coupling with acrylic acids or derivatives thereof, they may add to the β-carbon atom to form styrene derivatives or related compounds. They may be useful in reactions which are capable of being catalysed by palladium catalysts. Reaction conditions typically include temperatures of from about 20 to about 150° C. in a solvent. Temperatures may be about 20 to 100, 20 to 50, 50 to 150, 100 to 150 or 50 to 100° C., e.g. about 20, 30, 40, 50, 60, 70, 80, 90, 100, 110, 120, 130, 140 or 150° C. The catalysts are generally insensitive to protic solvents, so any solvent system capable of dissolving catalyst and reagents may be used. Depending on the particular reagents, suitable solvents include toluene, THF, DMF, DMSO, HMPT, HMPA, dioxane etc. The reactions are commonly conducted in the presence of a base. This may be for example a basic salt, such as a carbonate, phosphate etc. It may be a strong base such as hydride (in which case the solvent should not be protic). Reaction times will depend on the particular catalyst and reagents, as well as the temperature and, in some cases, the solvent. Typical reaction times are from about 1 to about 50 hours, and may be from 1 to 20, 1 to 10, 1 to 5, 5 to 50, 10 to 50, 20 to 50, 5 to 20, 10 to 20 or 5 to 10 hours, e.g. about 1, 2, 3, 4, 5, 6, 7, 8, 9, 10, 15, 20, 25, 30, 35, 40, 45 or 50 hours or more in some cases.

The catalysts of the present invention may be added to a reaction mixture, or a precursor thereto may be added together with a metal species capable of generating the active catalyst in situ. Thus, for example, a catalyst of formula I in which m is 1 or 2 may be used directly. This will typically be in cases where E is absent, i.e. the phosphorus atom is directly attached to the terphenyl nucleus. Alternatively, a ligand of formula I in which m is 0 (i.e. having no metal atom) may be used together with a metal species, e.g. a metal salt or metal complex, so as to form the catalyst of formula I in situ. In this case, the metal salt may be for example a chloride, a bromide, an iodide or some other salt of the metal and a suitable metal complex may be for example a dibenzylideneacetone complex. This will typically be in cases where E is present, commonly E is O, i.e. the phosphorus atom is attached to the terphenyl nucleus by E. The catalyst (or a precursor thereto) may be used in catalytic amounts. It may be used in an amount of about 0.1 to about 10% on a mole basis relative to the substrate and/or reagent(s). It may be used in an amount of about 0.1 to 5, 0.1 to 2, 0.1 to 1, 0.1 to 0.5, 0.5 to 10, 1 to 10, 2 to 10, 5 to 10, 0.5 to 5, 0.5 to 1, or 1 to 5% on a mole basis, e.g. about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 or 10% on a mole basis.

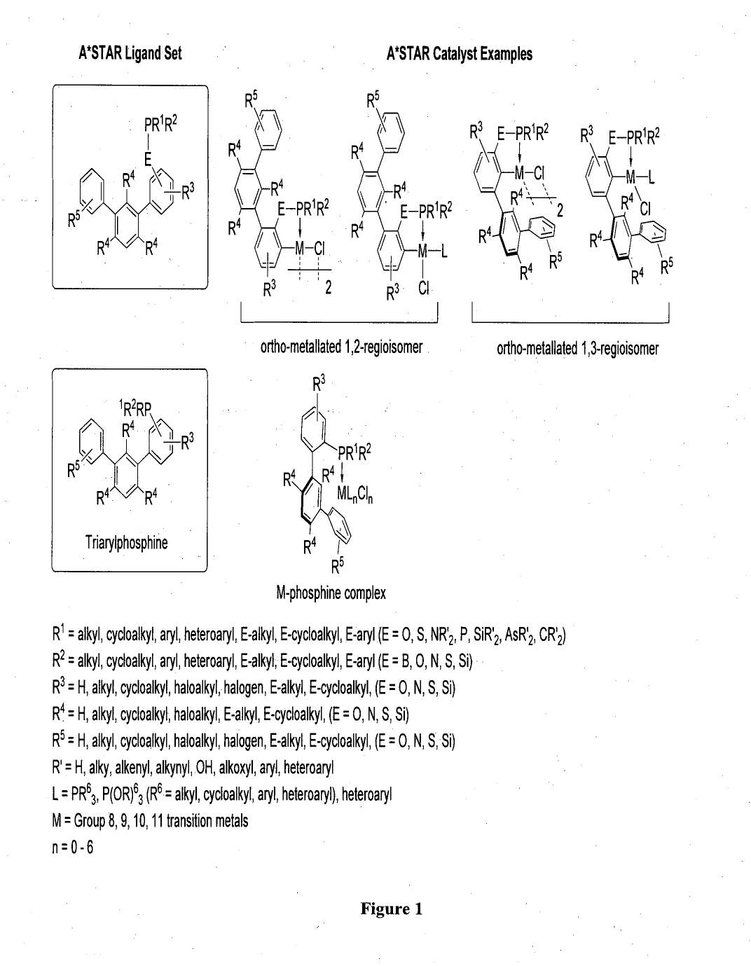

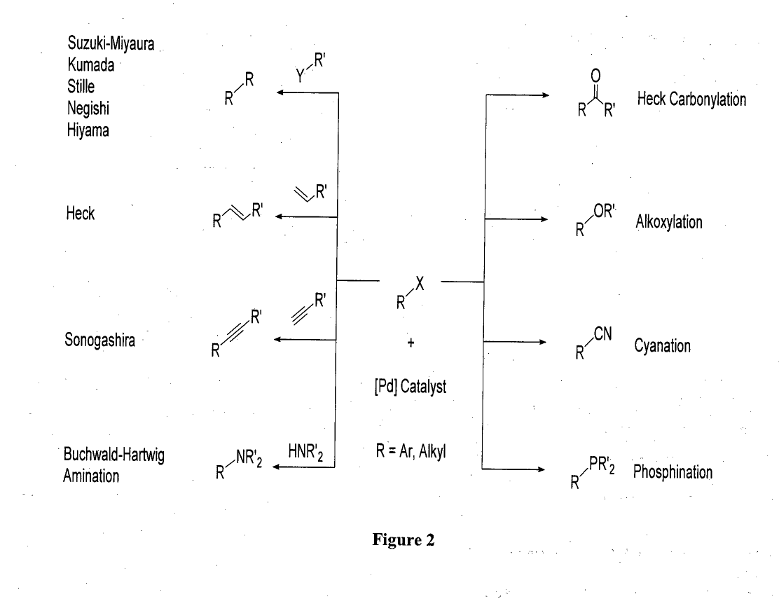

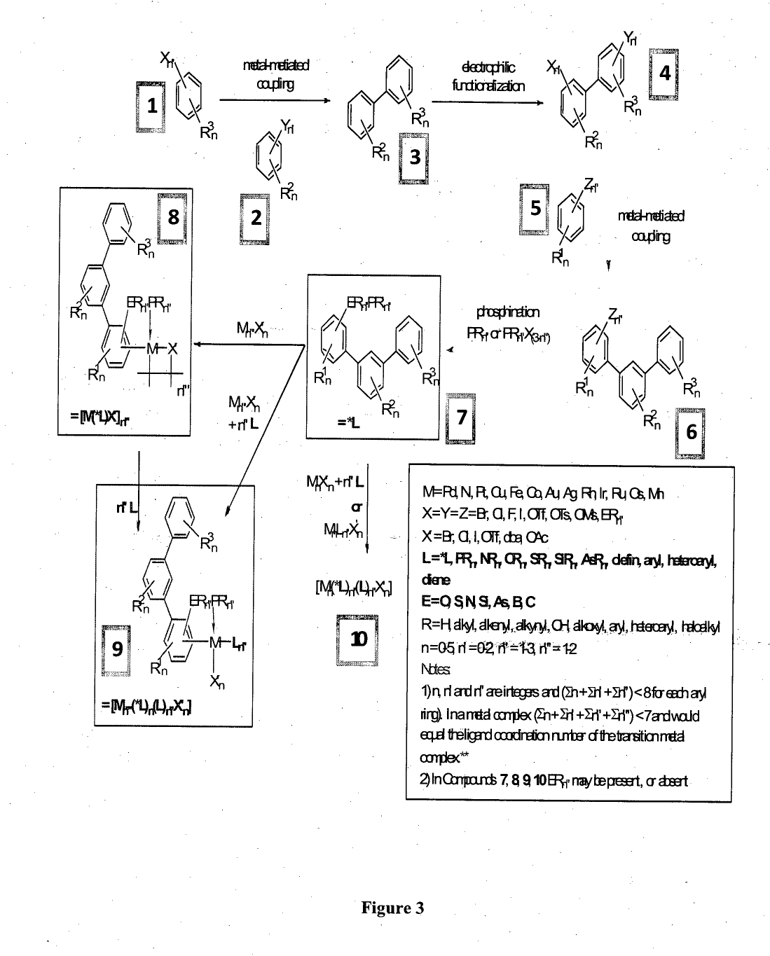

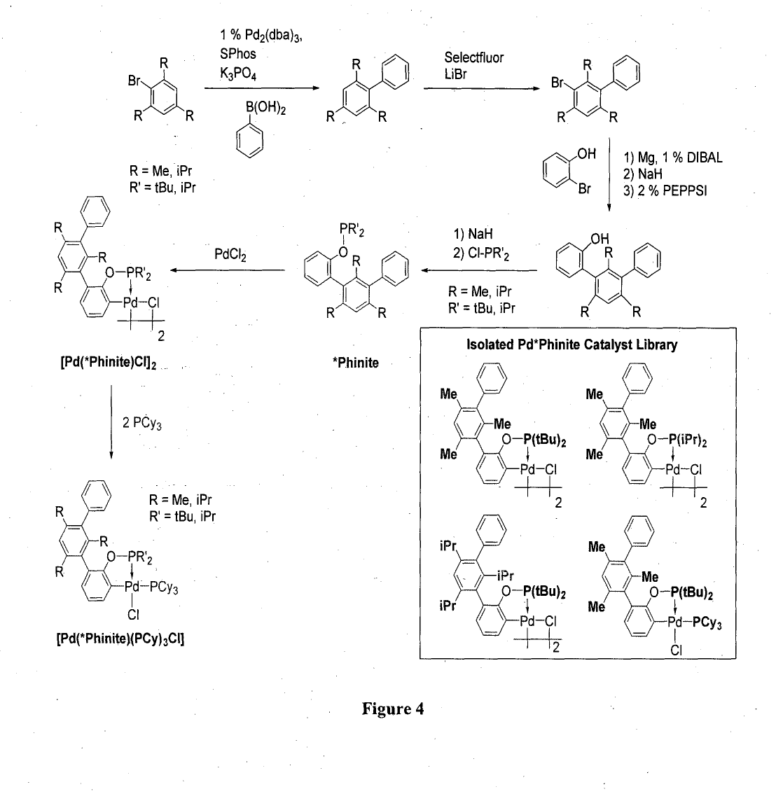

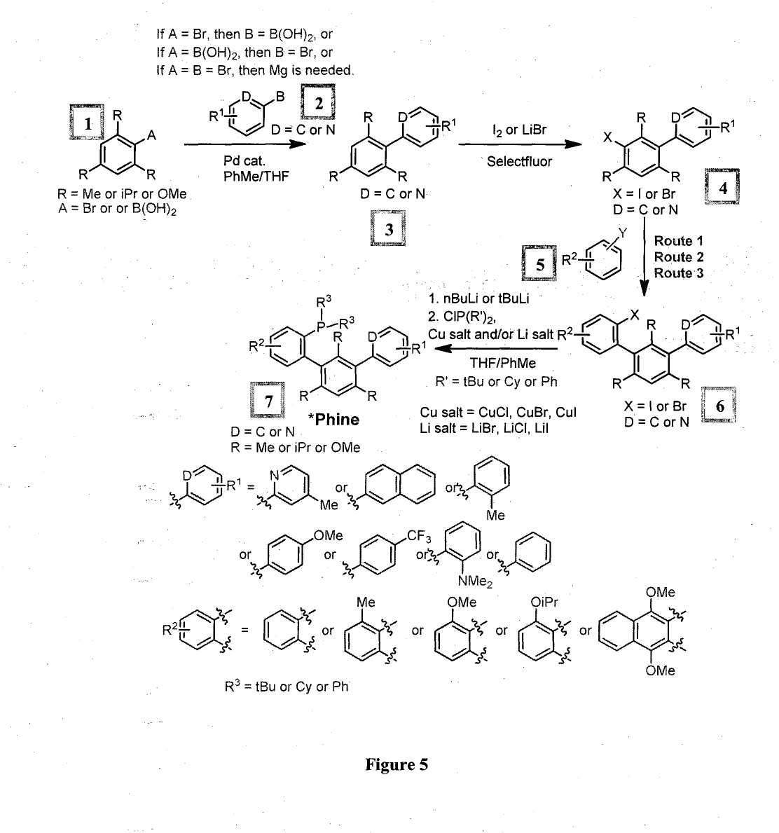

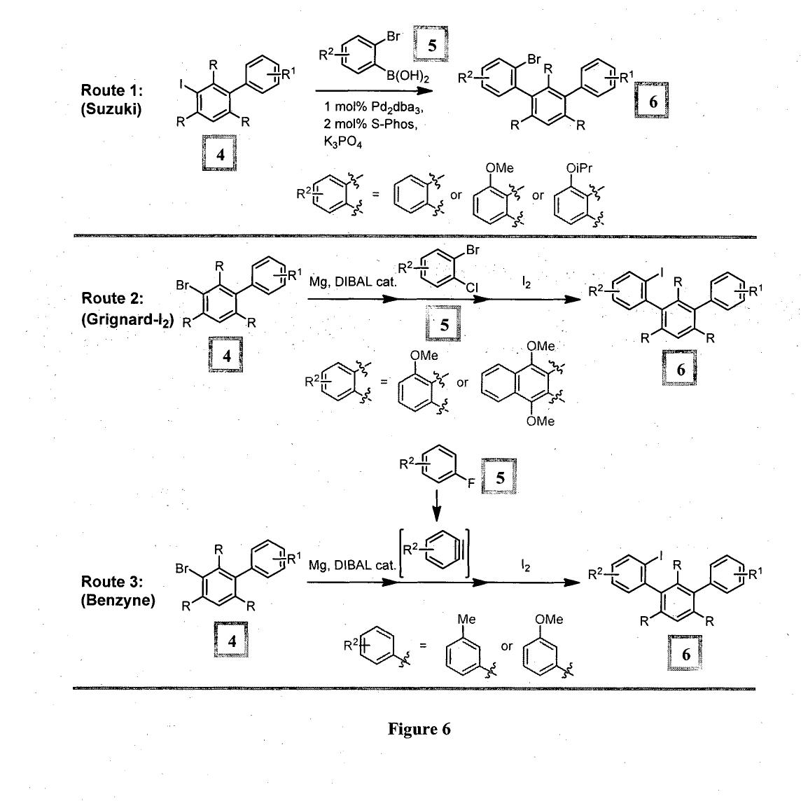

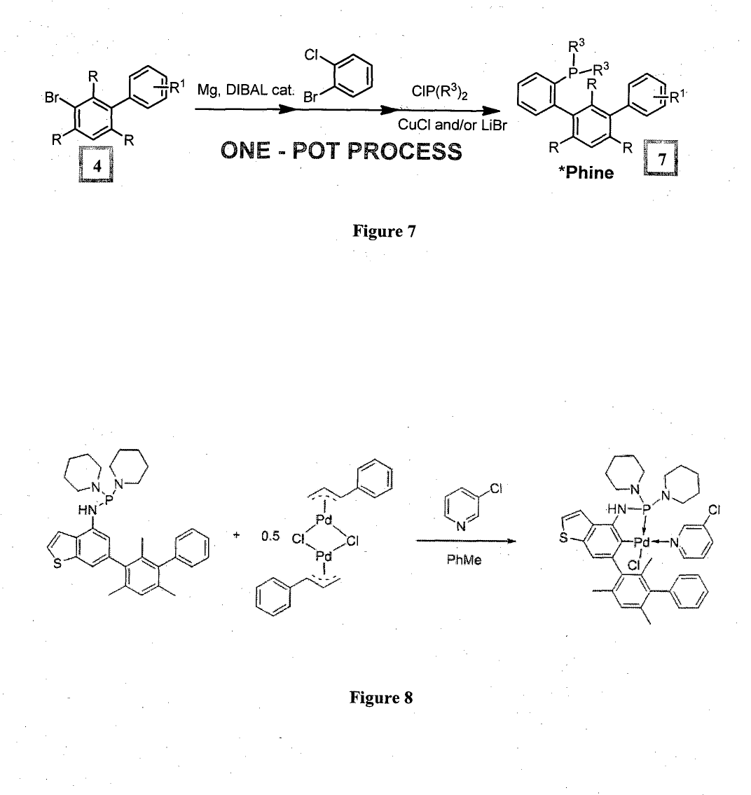

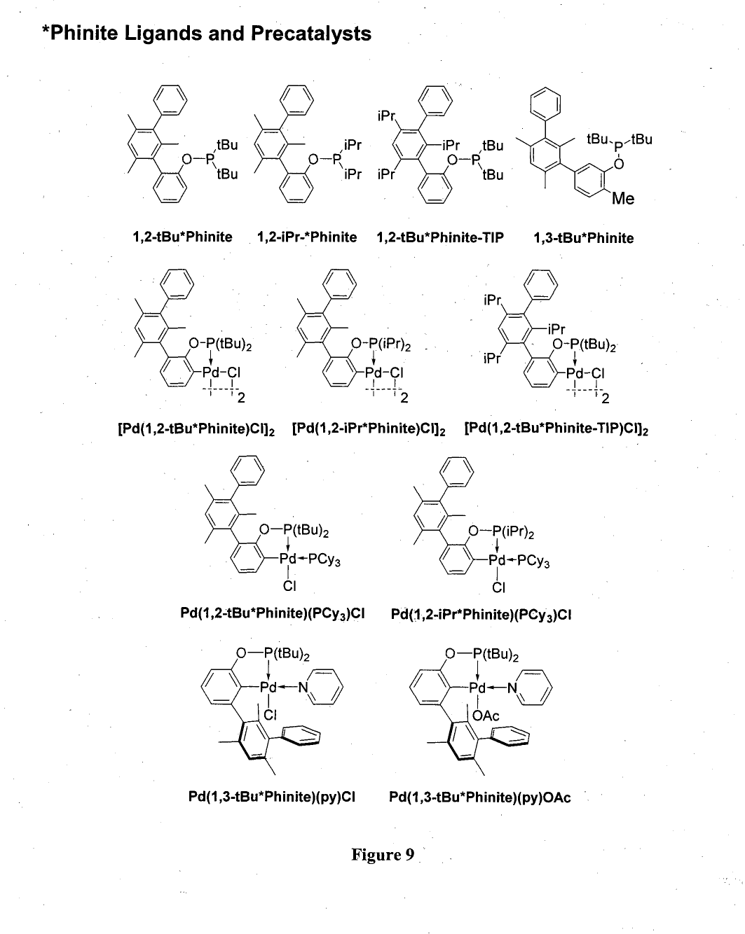

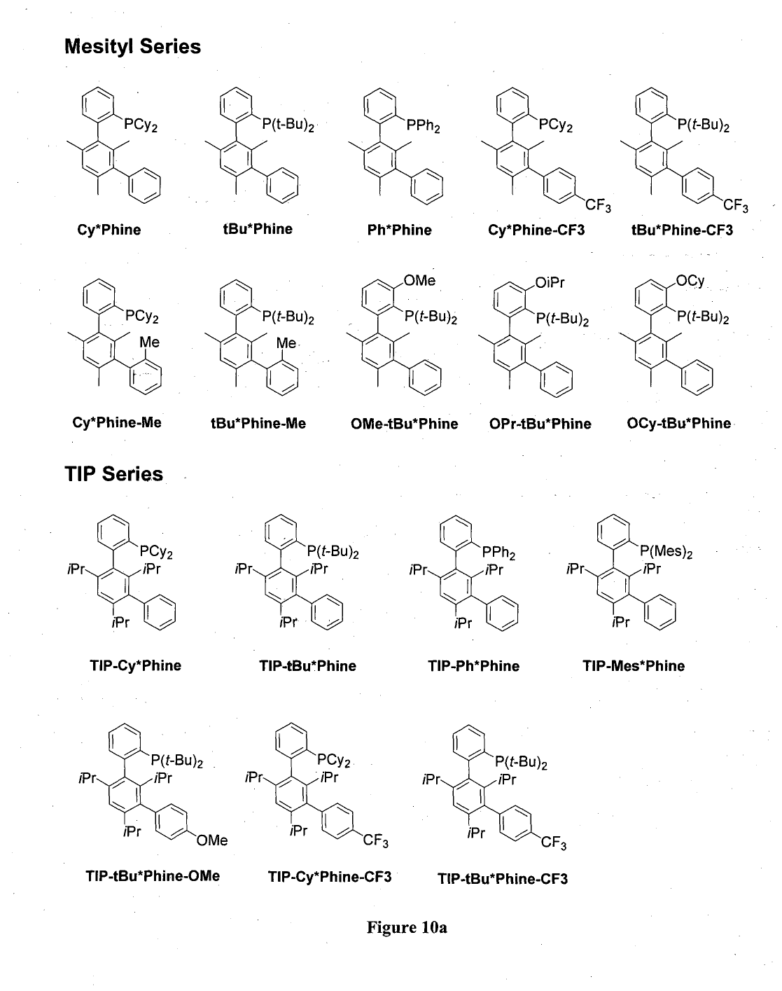

The invention described herein relates to the synthesis and development of catalysts incorporating terarylphosphinite, terarylphosphonite, terarylphosphite and terarylphosphine ligands for metal-mediated cross-coupling reactions. This invention encompasses the development of methodology to prepare a series of novel terarylphosphinite [Ar₃O—PR′₂], terarylphosphonite [Ar₃O—PR′(OR″)], terarylphosphite [Ar₃O—P(OR″)₂] and terarylphosphine [Ar₃PR′₂] ligand sets that possess versatility and tunability in both electronic and steric features (FIG. 1). These purpose-built teraryl ligands form highly active homogeneous catalysts when complexed to transition metals and are either competitive with, or superior to, the current class-leading catalytic systems for an array of cross-coupling transformations (FIG. 2) and capable of achieving outstanding TONs in excess of 10⁶ for a broad spectrum of substrates.

While Pd-mediated coupling reactions are very well known, there is a growing need for catalysts of higher efficiency and robustness to offset the rising commodity costs of precious metals. The catalyst systems of the present invention have been shown to be competitive with, and in numerous cases superior to, the most active catalysts known to date for Pd-mediated cross-coupling reactions such as the Suzuki-Miyaura transformation for a broad range of substrates. Furthermore, we have established the orthometallated catalyst systems' inherent stability to air and moisture, which enables reactions to be performed on the bench and free of inert atmosphere handling techniques. We have also demonstrated the system's effectiveness for coupling reactions in aqueous media, which is an attractive feature for biological applications and supports the increasingly important global green initiative. With high productivity, low imposing metal content, atom economy, low waste production and versatility coupled with air and moisture stability all rolled into one platform, the presently disclosed catalyst systems offer an attractive feature set with distinct advantages over existing technologies.

Representative catalytic performance results are outlined in Tables 1 to 4 using [Pd(1,2-tBu*Phinite)Cl]₂, TIP-Cy*Phine/Pd₂(dba)₃ and TIP-Cy*Phine/Pd(OAc)₂. Table 1 compares the performance of a selected catalyst according to the present inventions (“A*STAR Catalyst” [Pd(1,2-tBu*Phinite)Cl]₂) with that of a known catalyst (“Bedford”). It can be seen that the catalyst of the present invention is superior in yield and turn-over number under comparable conditions. Table 2 shows similar comparisons for two catalysts according to the present invention compared with two prior art catalysts. This indicates that the catalyst should be tailored for the particular reaction, since different catalysts within the scope of the present invention may provide improved performance. Table 3 shows a similar comparison between different catalysts according to the present invention against the commercially available and well established Pd₂(dba)₃/SPhos combination for sterically hindered substrates. This indicates that conditions and catalysts may be specifically matched in order to provide desired performance for a particular coupling reaction. Table 4 specific examples of reactions using the catalysts that typically exhibit poor reactivity in metal catalyzed cross-coupling reactions using Pd catalysts with phosphinite and phosphine-based ligands. Here, the capability of the present catalyst system is demonstrated, as well as its potential to outperform leading existing technologies.

TABLE 1
Comparative Suzuki-Miyaura coupling results between class-leading
Bedford catalyst and a representative catalyst according to the present
invention.

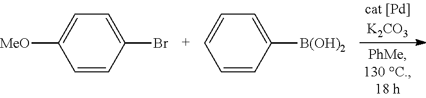

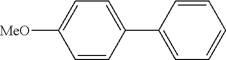 [SMILES: COc1ccc(-c2ccccc2)cc1]

| | | Yield | Yield | |
|---|---|---|---|---|
| | | (%) | (%) | TON |
| | Load | (ArAr, | (ArAr, | (ArAr, |
| Catalyst | (mol %) | GC) | isol) | GC) |
| Bedford | 0.01 | 96 | 84 | 9,600 |
| [Pd(1,2- | 0.01 | >99 | 96 | 10,000 |
| tBu*Phinite)Cl]₂ | | | | |
| Bedford | 0.0001 | 16 | N/A | 160,000 |
| [Pd(1,2- | 0.0001 | 51 | N/A | 510,000 |
| tBu*Phinite)Cl]₂ | | | | |
| Bedford | 0.00001 | 10.5 | N/A | 1.0*10⁶ |
| [Pd(1,2- | 0.00001 | 16.0 | N/A | 1.6*10⁶ |
| tBu*Phinite)Cl]₂ | | | | |

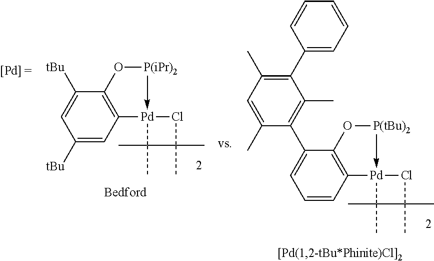

TABLE 2
Representative substrate scope and applicability of a representative
catalyst according to the present invention compared to state-of-
the-art commercial competitors.

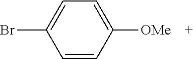

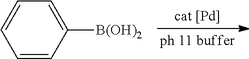

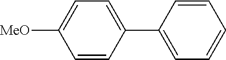 [SMILES: COc1ccc(-c2ccccc2)cc1]

| | | | | Yield (%) | TON |
|---|---|---|---|---|---|
| | Load | | | (ArAr, | (ArAr, |
| Catalyst | (mol %) | T (° C.) | t(h) | GC) | GC) |
| Bedford 2 | 0.02 | 30 | 18 | 14 | 700 |
| Bedford 1 | 0.02 | 30 | 18 | 39 | 1850 |
| [Pd(1,2-tBu*Phinite)Cl]₂ | 0.02 | 30 | 18 | 19 | 950 |
| [Pd(1,2-iPr*Phinite)Cl]₂ | 0.02 | 30 | 18 | 54 | 2700 |
| Bedford 2 | 0.01 | 90 | 8 | 89 | 8900 |
| Bedford 1 | 0.01 | 90 | 8 | 91 | 9100 |
| [Pd(1,2-tBu*Phinite)Cl]₂ | 0.01 | 90 | 8 | 87 | 8700 |
| [Pd(1,2-iPr*Phinite)Cl]₂ | 0.01 | 90 | 8 | 83 | 8300 |

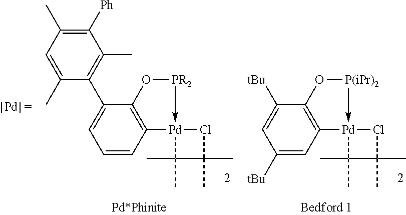

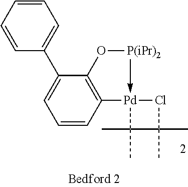

TABLE 3
Comparison of different A*STAR catalysts according to the
invention against the commercially available and well
established Pd₂(dba)₃/SPhos combination.

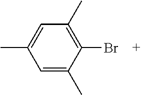

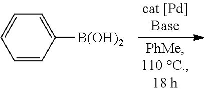

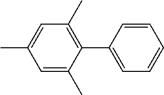

| En- | | | Load | Yield | |
|---|---|---|---|---|---|
| try | Catalyst | Base | (%) | (%) | TON |
| 1 | [Pd(1,2-tBu*Phinite)Cl]₂ | K₂CO₃ | 0.01 | 33 | 3300 |
| 2 | [Pd(1,2-iPr*Phinite)Cl]₂ | K₃PO₄ | 0.1 | >99 | 1000 |
| 3 | Pd₂(dba)₃, TIP-Cy*Phine | K₃PO₄ | 1 | >99 | 100 |
| 4 | Pd₂(dba)₃, SPhos | K₃PO₄ | 1 | >99 | 100 |

| | |
|---|---|
| 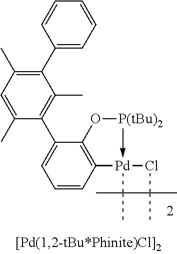 | A |
| 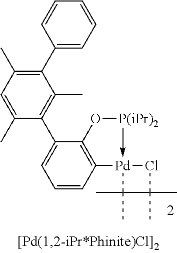 | B |
| 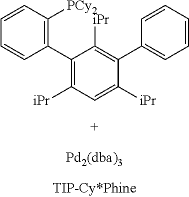 | C |
| 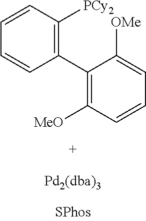 | D |

TABLE 4
Examples of coupling reactions with inventive catalysts compared to the state-of-the art (“Bedford”).

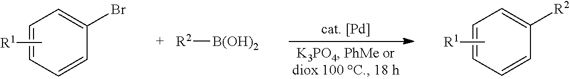

| En- | | Load | | | | |
|---|---|---|---|---|---|---|
| try | Catalyst | (%) | Ar—X | R²—B(OH)₂ | Ar—R² | Yield (%) |
| 1 | Pd(OAc)₂, TIP- Cy*Phine | 2 | 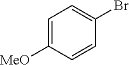 | 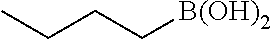 | 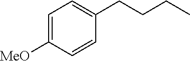 | 57 |
| 2 | Bedford | 2 | 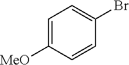 | 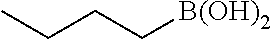 | 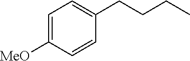 | 25 |

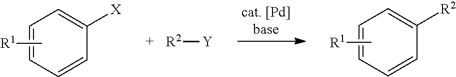

| En- | | Cat | | | | | | T | Yield |
|---|---|---|---|---|---|---|---|---|---|
| try | Catalyst | (%) | Ar—X | R—Y | Ar—Y | Base | Solv | (°C.) | (%) |
| 1 | [Pd(1,2- tBu*Phinite)Cl]₂ | 2 | 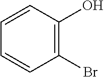 | 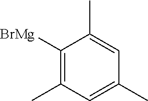 | 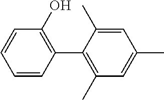 | NaH | THF | 70 | 70 |
| 2 | Pd₂(dba)₃, TIP-Cy*Phine | 2 | 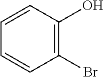 | 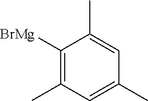 | 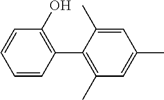 | NaH | THF | 70 | 40 |
| 3 | [Pd(1,2- tBu*Phinite)Cl]₂ | 1 | 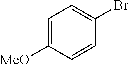 | 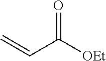 | 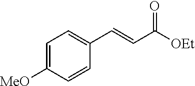 | K₃PO₄ | DMF | 130 | 92 |
| 4 | Bedford | 1 | 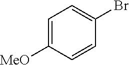 | 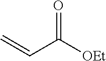 | 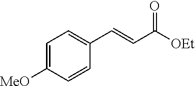 | K₃PO₄ | DMF | 130 | 23 |
| 5 | Pd₂(dba)₃, TIP-Cy*Phine | 1 | 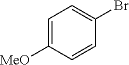 | 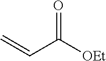 | 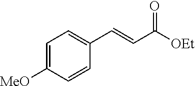 | K₃PO₄ | DMF | 130 | 64 |
| 6 | Pd₂(dba)₃, SPhos | 1 | 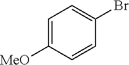 | 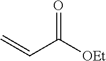 | 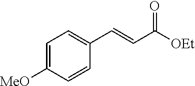 | K₃PO₄ | DMF | 130 | 70 |
| 7 | Pd₂(dba)₃, TIP-Cy*Phine | 4 | 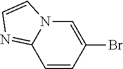 | 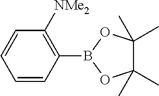 | 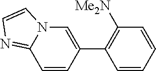 | K₃PO₄ | Diox/ H₂O | 100 | >99 |

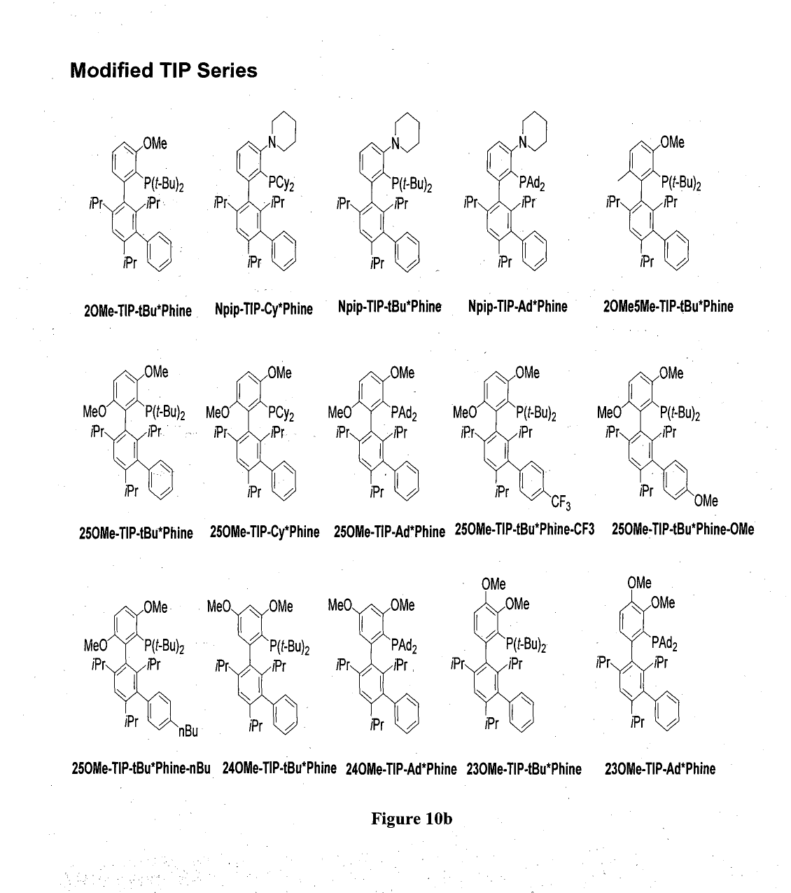

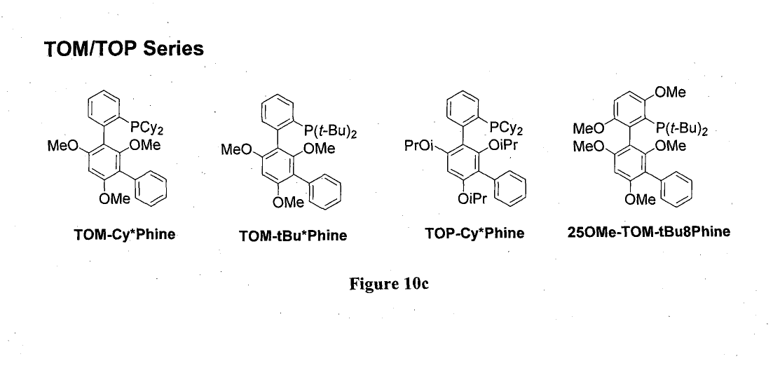

In embodiments of the invention there are provided complexes of formula (TPdCl)₂ and T₂PdCl₂, wherein each T independently is any one of the ligands shown in FIGS. 10a to 10c.

It is considered that the use of the meta-terphenyl nucleus provides hitherto unrecognised reactivity for the compounds of the present invention. In particular, the 1,2,3,4,6-substitution pattern of the central ring (having positions 1 and 3 aryl substituted) provides excellent catalyst performance. The specific teraryl ring system with this substitution pattern has not previously been synthesised.

It is considered that industries such as pharmaceuticals, agrochemicals, polymers and new materials that utilize fine chemicals will find substantial utility for this invention. The installation of an improved catalyst system into synthetic processes in these industries would be facile and facilitate margin expansion generating greater returns to their stakeholders.

### EXAMPLES

FIGS. 10a to 10c shows the nomenclature of compounds used or synthesised in the Examples set out below. The Examples show Experimental and Synthetic Data for *Phine *Phinite Ligand, Pd*Phine and Pd*Phinite Precatalysts and Related Complexes.

### Mesityl Series

### Synthesis of Ligand Cy*Phine

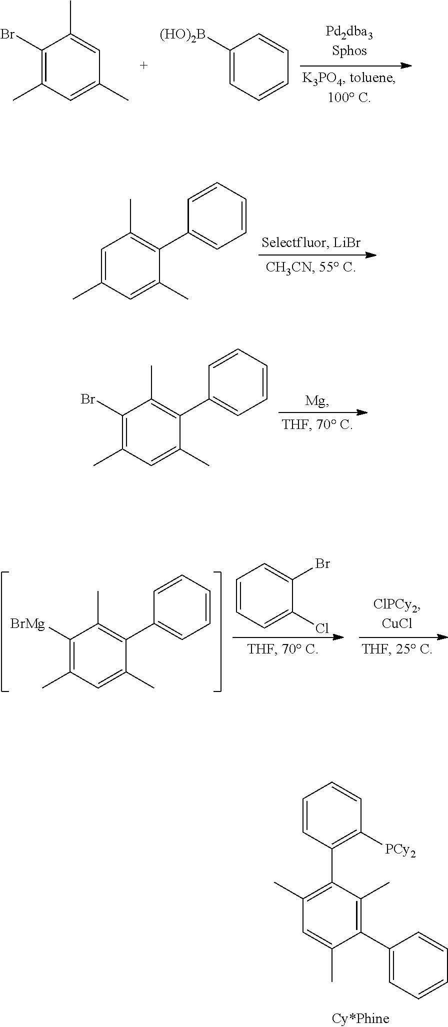

### 2,4,6-Trimethyl-1,1′-biphenyl

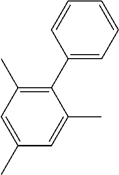

To an oven dried 2 neck 50 mL flask equipped with a magnetic stir bar and condenser, 1.98 g (10 mmol) of mestlyene bromide, 2.44 g (15 mmol) of phenylbornic acid, 4.224 g (20 mmol) of potassium phosphate tribasic, 0.820 g (2 mol %) of SPhos and 0.910 g (1 mol %) of tris(dibenzylideneacetone)dipalladium(0) was added. The mixture was then evacuated and backfilled with argon three times then anhydrous tetrahydrofuran (10 mL) and toluene (10 mL) were added to the flask. The mixture was stirred at 60° C. for 16 h then cooled to room temperature prior to filtration through a pad of Celite, washing thoroughly with EtOAc. The red filtrate was concentrated in vacuo yielding a crude residue that was purified by flash chromatography on silica gel using hexanes as the eluent to afford a colorless liquid (1.56 g, 80%). ¹H (600 MHz, CDCl₃) δ: 7.59 (t, J=7.44 Hz, 2H), 7.51-7.49 (m, 1H), 7.33 (dd, J=6.84 Hz, 1.38 Hz, 2H), 7.14 (s, 2H), 2.53 (s, 3H), 2.21 (s, 6H). ¹³C (150 MHz, CDCl₃) δ: 141.34, 139.28, 136.69, 136.11, 129.49, 128.58, 128.28, 126.71, 21.24, 20.95.

### 3-Bromo-2,4,6-trimethyl-1,1′-biphenyl

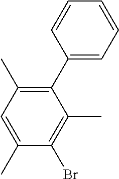 [SMILES: Cc1cc(C)c(-c2ccccc2)c(C)c1Br]

To a 250 mL flask wrapped with aluminium foil equipped with a magnetic bar was charged with 1.96 g (10 mmol) of 2,4,6-trimethyl-1,1′-biphenyl, 0.869 g (10 mmol) of lithium bromide, 3.98 g (11 mmol) of selectfluor and acetonitrile (70 mL). The mixture was stirred at room temperature for 16 h after which the solvent was removed under vacuum. The residue was extracted into dichloromethane and filtered through a pad of Celite. The filtrate was collected and washed with concentrated sodium thiosulfate. The organic layer was collected, dried with MgSO₄, filtered and concentrated to dryness under reduced pressure. The crude residue was then purified by flash chromatography on silica gel using hexanes and the eluent to afford a colorless liquid (0.220 g, 80%). ¹H (600 MHz, CDCl₃) δ: 7.42 (t, J=7.3 Hz, 2H), 7.35-7.33 (m, 1H), 7.09 (dd, J=6.84, 1.44 Hz, 2H), 7.02 (s, 1H), 2.43 (s, 3H), 2.12 (s, 3H), 1.93 (s, 3H). ¹³C (150 MHz, CDCl₃) δ: 141.45, 141.11, 137.13, 136.25, 135.01, 129.62, 129.32, 128.73, 127.07, 125.60, 24.20, 22.19, 20.81.

### Dicyclohexyl(2′,4′,6′-trimethyl-[1,1′:3′,1″-terphenyl]-2-yl)phosphane (Cy*Phine)

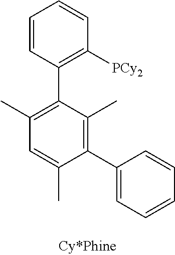

To an oven-dried flask, Mg turnings (54.8 mg, 2.2 mmol, 2.2 equiv.), 1 mL THF and 3 drops of iBu₂AlH were added subsequently. After 5 min of stirring, 3-bromo-2,4,6-trimethyl-1,1′-biphenyl (274 mg, 1 mmol, 1.0 equiv) was added slowly. The flask was capped under nitrogen and the mixture was then heated to 70° C. in a preheated oil bath with vigorous stirring for 2 h prior to the dropwise addition of 2-bromochlorobenzene (211 mg, 1.1 mmol, 1.1 equiv) at 70° C., after which the mixture was stirred for another 3 h. At this time, the mixture was cooled to room temperature and was transferred to another oven-dried flask with charge of CuCl (5 mg, 0.05 mmol, 5 mol %). Additional of 1 mL anhydrous THF was rinsed and also was transferred. PCy₂Cl (233 mg, 1.1 mmol, 1.1 equiv) was subsequently added at room temperature and stirred overnight. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (234 mg, 50%), herein referred to as Cy*Phine. ¹H NMR (400 MHz, CDCl₃) δ 7.70 (dd, J=5.0, 2.4 Hz, 3H), 7.51-7.41 (m, 3H), 7.38 (s, 1H), 7.31 (d, J=7.2 Hz, 2H), 7.26 (s, 1H), 2.41 (s, 3H), 2.29 (s, 3H), 2.20 (s, 3H), 2.15-1.60 (m, 10H), 1.60-1.04 (m, 12H) ppm. ¹³C NMR (151 MHz, CDCl₃) δ 148.53 (d, J_{C-P}=30.3 Hz), 142.04, 139.55, 139.35 (d, J_{C-P}=6.1 Hz), 135.55 (d, J_{CP}=18.9 Hz), 134.63 (d, J=11.9 Hz), 133.80, 132.56 (d, J_{CP}=3.0 Hz), 130.59 (d, J_{CP}=5.8 Hz), 129.32 (d, J_{CP}=15.8 Hz), 128.63, 128.48, 128.35, 127.95, 126.30, 126.13, 34.75 (d, J_{CP}=15.5 Hz), 32.88 (d, J_{CP}=13.8 Hz), 30.67 (d, J_{CP}=12.1 Hz), 30.06 (dd, J_{CP}=26.9, 14.9 Hz), 29.18 (d, J_{CP}=10.8 Hz), 27.72 (dd, J_{CP}=14.7, 9.4 Hz), 27.28 (d, J_{CP}=10.2 Hz), 26.49 (d, J_{CP}=17.9 Hz), 21.49 (d, J_{CP}=6.1 Hz), 20.95, 20.01 ppm. ³¹P NMR (243 MHz, CDCl₃) δ=−8.75 ppm.

### Synthesis of Ligand tBu*Phine

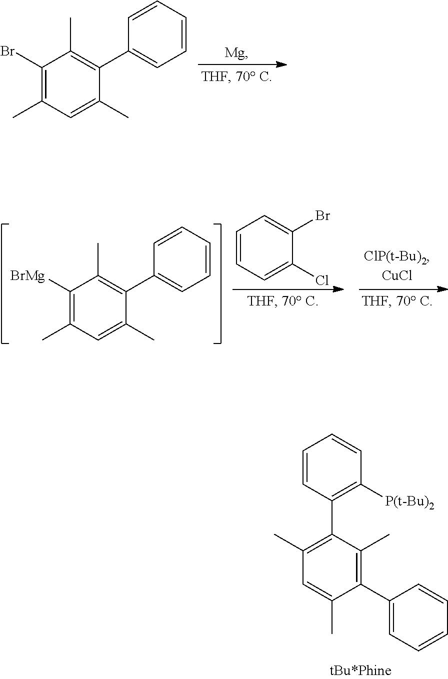

### Di-tert-butyl(2′,4′,6′-trimethyl-[1,1′:3′,1″-terphenyl]-2-yl)phosphane (tBu*Phine)

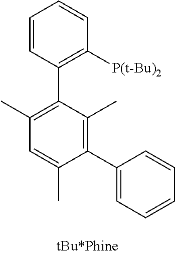

To an oven-dried flask, Mg turnings (105.6 mg, 4.4 mmol, 2.2 equiv.), 1 mL THF and 6 drops of iBu₂AlH were added subsequently. After 5 min of stirring, 3-bromo-2,4,6-trimethyl-1,1′-biphenyl (548 mg, 2 mmol, 1.0 equiv.) was added slowly. The mixture was heated to 70° C. in a preheated oil bath for 2 h with vigorous stirring prior to the addition of 2-bromochlorobenze (473.0 mg, 2.4 mmol, 1.2 equiv.). The mixture was stirred at 70° C. for another 3 h. At this time, the reaction mixture was cooled down to room temperature. Then the reaction mixture was transferred to a new oven-dried flask using syringe to remove remaining Mg turnings, and anhydrous CuCl (214.4 mg, 2.0 mmol, 1.0 equiv.) was added and stirred 15 min. To this solution, ClP^{t}Bu₂ (361.3 mg, 2.0 mmol, 1.0 equiv.) was added via syringe slowly. The resulting mixture was allowed to reach 70° C. and stirred at this temperature for 15 h. The reaction mixture was cooled down to room temperature and diluted with ethyl acetate. 5 mL 28-30% NH₄OH was added to wash till the organic layer colourless (at least 5 mL×4). The organic layer was washed with brine and dried with MgSO₄, filtered and concentrated to give the crude product; recrystallization in MeOH affords pure white solid (252 mg, 30%), labelled as tBu*Phine. ¹H NMR (600 MHz, Benzene-d₆), δ 8.02 (d, J=7.4 Hz, 1H), 7.44 (dd, J=18.7, 8.3 Hz, 3H), 7.32 (m, 2H), 7.22 (s, 1H), 2.41 (s, 3H), 2.27 (s, 3H), 2.20 (s, 3H), 1.38 (dd, J=21.0, 11.2 Hz, 18H) ppm. ¹³C NMR (151 MHz, Chloroform-d) δ 149.43 (d, J_{CP}=33.1 Hz), 142.14, 139.73, 139.45, 136.85 (d, J_{CP}=29.5 Hz), 136.17, 134.54, 134.25 (d, J_{CP}=38.1 Hz), 131.47 (d, J_{CP}=7.0 Hz), 129.29 (d, J_{CP}=15.4 Hz), 128.41 (d, J_{CP}=24.8 Hz), 126.25, 125.46, 32.67 (dd, J_{CP}=24.7, 7.2 Hz), 31.40 (d, J_{CP}=15.0 Hz), 30.61 (d, J_{CP}=15.0 Hz), 22.12 (d, J_{CP}=5.8 Hz), 20.86 ppm. ³¹P NMR (243 MHz, CDCl₃) δ=24.14 ppm.

### Synthesis of Ligand Cy*Phine-CF3

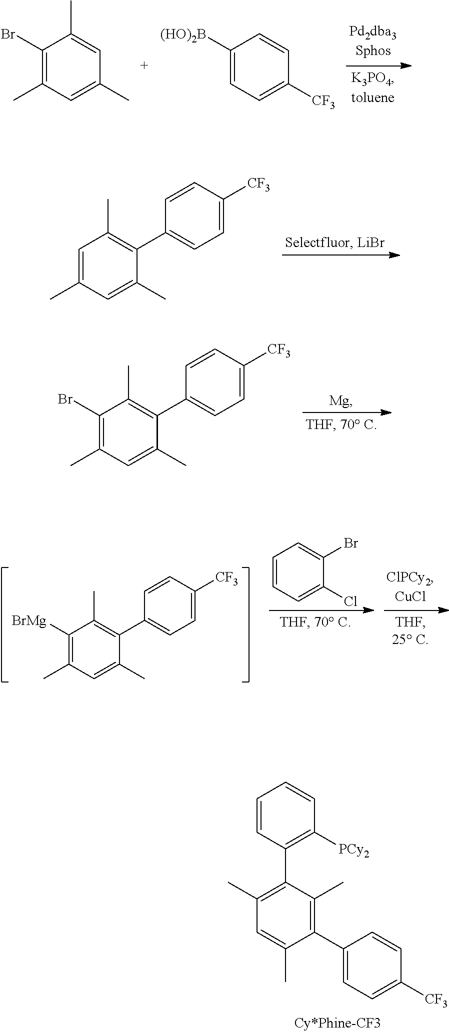

### 2,4,6-trimethyl-4′-(trifluoromethyl)-1,1′-biphenyl

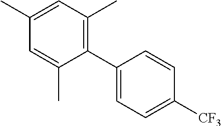 [SMILES: Cc1cc(C)c(-c2ccc(C(F)(F)F)cc2)c(C)c1]

4.9 g (25 mmol) of mestlyene bromide, 5.2 g (27 mmol) of p-CF₃-phenylbornic acid, 10.6 g (20 mmol) of K₃PO₄, 0.411 g (2 mol %) of SPhos and 0.458 g (1 mol %) of Pd₂(dba)₃ was added to an oven dried 50 mL flask. The mixture was then vacuumed for 5 min and flush with argon gas. 10 mL of dry THF and 10 mL of dry toluene was added to the flask. The reaction was then heated at 60° C. for 16 hr under argon. The mixture was then cooled to room temperature and filters through silica and washes with Ethyl acetate. The filtrates were collect and remove solvent with vacuum. The crude is purified by column with Hexane as eluent to get colorless oil (6.1. g, 92.4%). ¹H NMR (600 MHz, Chloroform-d) δ 7.71 (d, J=7.8 Hz, 2H), 7.30 (d, J=7.6 Hz, 2H), 6.99 (s, 1H), 2.37 (s, 3H), 2.02 (s, 6H) ppm. ¹³C NMR (151 MHz, Chloroform-d) δ 145.03, 137.61, 137.23, 135.62, 129.78, 128.24, 125.39 (d, J=3.8 Hz), 21.03, 20.67 ppm.

### 3-bromo-2,4,6-trimethyl-4′-(trifluoromethyl)-1,1′-biphenyl

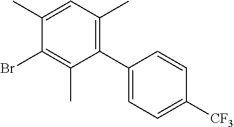 [SMILES: Cc1cc(C)c(-c2ccc(C(F)(F)F)cc2)c(C)c1Br]

5.0 g (19 mmol) of 2,4,6-trimethyl-4′-(trifluoromethyl)-1,1′-biphenyl, 7.71 g (22.8 mmol) of lithium bromide, 1.81 g (21 mmol) of selectfluor and 70 mL of MeCN was added to a 250 mL flask and stirred 55° C. for 16 hr. The solvent was removed and DCM was added to the crude. The mixture was filtered; the filtrate was wash with sodium thiosulfate. The organic layer was collected and dry with MgSO₄. The crude was then purified with column with Hexane as eluents to get a white solid (5.4 g, 83.3%). ¹H NMR (600 MHz, Chloroform-d) δ 7.73 (d, J=8.0 Hz, 2H), 7.27 (d, J=8.0 Hz, 2H), 7.07 (s, 1H), 2.47 (s, 3H), 2.14 (s, 3H), 1.95 (s, 3H) ppm. ¹³C NMR (151 MHz, CDCl₃) δ 145.03, 139.36, 137.63, 135.70, 134.41, 129.63, 125.59, 125.56, 125.54, 24.01, 21.97, 20.54 ppm.

### Dicyclohexyl(2′,4′,6′-trimethyl-4″-(trifluoromethyl)-[1,1′:3′,1″-terphenyl]-2-yl)phosphane (Cy*Phine-CF3)

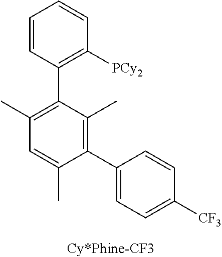

To an oven-dried flask, Mg turnings (54.8 mg, 2.2 mmol, 2.2 equiv.), 1 mL THF and 3 drops of iBu₂AlH were added subsequently. After 5 min of stirring, 3-bromo-2,4,6-trimethyl-4′-(trifluoromethyl)-1,1′-biphenyl (342 mg, 1 mmol, 1.0 equiv) was added slowly. The flask was capped under nitrogen and the mixture was then heated to 70° C. in a preheated oil bath with vigorous stirring for 2 h prior to the dropwise addition of 2-bromochlorobenzene (211 mg, 1.1 mmol, 1.1 equiv) at 70° C., after which was stirred for another 3 h. At this time, the mixture was cooled to room temperature and was transferred to another oven-dried flask with charge of CuCl (5 mg, 0.05 mmol, 5 mol %). Additional of 1 mL anhydrous THF was rinsed and also was transferred. PCy₂Cl (233 mg, 1.1 mmol, 1.1 equiv) was subsequently added at room temperature and stirred 24 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (306 mg, 57%), labelled as Cy*Phine-CF3. ¹H NMR (400 MHz, Chloroform-d) δ 7.70 (d, J=7.9 Hz, 1H), 7.58 (d, J=7.3 Hz, 1H), 7.42-7.14 (m, 6H), 7.06 (s, 1H), 2.03 (d, J=12.6 Hz, 6H), 1.66 (m, 13H), 1.12 (m, 9H) ppm. ¹³C NMR (151 MHz, Chloroform-d) δ 148.20 (d, J_{CP}=30.4 Hz), 139.61 (d, J_{CP}=6.1 Hz), 135.71-135.02 (m), 130.51 (d, J_{CP}=5.8 Hz), 125.46 (dd, J_{CP}=13.8, 3.8 Hz), 34.67 (d, J_{CP}=15.3 Hz), 33.06 (d, J_{CP}=13.7 Hz), 30.62 (d, J_{CP}=12.0 Hz), 30.05 (dd, J_{CP}=28.8, 14.5 Hz), 29.30 (d, J_{CP}=11.3 Hz), 27.68 (dd, J_{CP}=15.7, 9.6 Hz), 27.46-26.95 (m), 26.46 (d, J_{CP}=17.5 Hz), 21.49 (d, J_{CP}=5.7 Hz), 20.85, 19.97 ppm. ³¹P NMR (243 MHz, CDCl₃) δ=−8.72 ppm. ¹⁹F NMR (376 MHz, CDCl₃) δ=−62.32 ppm.

### Synthesis of Ligand tBu*Phine-CF3

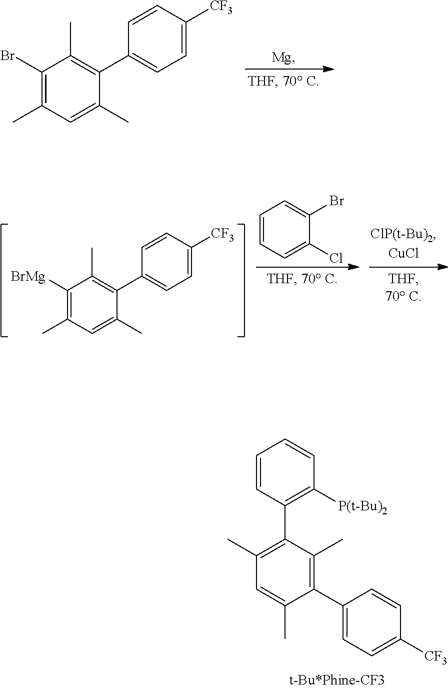

### Di-tert-butyl(2′,4′,6′-trimethyl-4″-(trifluoromethyl)-[1,1′:3′,1″-terphenyl]-2-yl)phosphane (tBu*Phine-CF3)

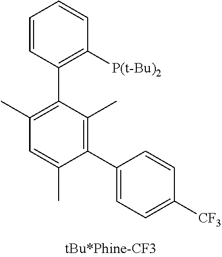

To an oven-dried flask, Mg turnings (105.6 mg, 4.4 mmol, 2.2 equiv.), 1 mL THF and 6 drops of iBu₂AlH were added subsequently. After 5 min of stirring, 3-bromo-2,4,6-trimethyl-4′-(trifluoromethyl)-1,1′-biphenyl (684 mg, 2 mmol, 1.0 equiv.) was added slowly. The flask was capped under nitrogen and the mixture was heated to 70° C. with vigorous stirring in a preheated oil bath for 2 h prior to the addition of 2-bromochlorobenze (473.0 mg, 2.4 mmol, 1.2 equiv.). The mixture was stirred at 70° C. for another 3 h. At this time, the reaction mixture was cooled down to room temperature. Then the reaction mixture was transferred to a new oven-dried flask using syringe to remove remaining Mg turnings, and anhydrous CuCl (214.4 mg, 2.0 mmol, 1.0 equiv.) was added and stirred 15 min. To this solution, ClP^{t}Bu₂ (361.3 mg, 2.0 mmol, 1.0 equiv.) was added via syringe slowly. The resulting mixture was allowed to reach 70° C. and stirred at this temperature for 16 h. The reaction mixture was cooled down to room temperature and diluted with ethyl acetate. 5 mL 28-30% NH₄OH was added to wash till the organic layer colourless (at least 5 mL×4). The organic layer was washed with brine and dried with MgSO₄, filtered and concentrated to give the crude product; recrystallization in MeOH affords pure white solid (339 mg, 35%), labelled as tBu*Phine-CF3. ¹H NMR (400 MHz, Benzene-d₆) δ 8.03 (d, J=6.9 Hz, 1H), 7.59 (dd, J=7.3, 4.7 Hz, 3H), 7.36-7.23 (m, 3H), 7.18 (s, 1H), 7.16-7.08 (m, 1H), 2.37 (s, 3H), 2.09 (s, 3H), 2.05 (s, 3H), 1.38 (d, J=11.3 Hz, 18H) ppm. ¹³C NMR (151 MHz, Benzene-d₆) δ 149.61 (d, J_{CP}=33.6 Hz), 146.22, 140.19 (d, J_{CP}=5.8 Hz), 138.11, 137.21 (d, J_{CP}=31.0 Hz), 136.26, 135.52, 133.92, 133.58, 131.55 (d, J_{CP}=6.7 Hz), 129.89 (d, J_{CP}=31.0 Hz), 128.98, 128.70, 125.74, 125.55 (dd, J_{CP}=20.1, 3.7 Hz), 32.55 (dd, J_{CP}=26.0, 9.4 Hz), 30.92 (dd, J_{CP}=24.7, 15.3 Hz), 22.09 (d, J_{CP}=4.1 Hz), 21.05 (d, J_{CP}=4.7 Hz), 20.52 ppm. ³¹P NMR (243 MHz, CDCl₃) δ=23.69 ppm. ¹⁹F NMR (376 MHz, CDCl₃) δ=−62.01 ppm.

### Synthesis of Ligand Ph*Phine

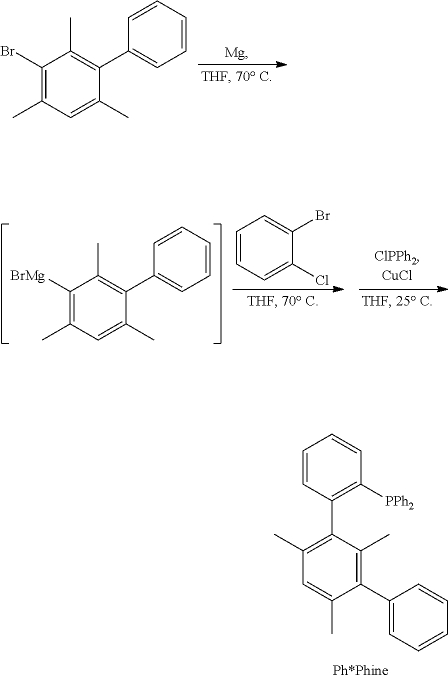

### Diphenyl(2′,4′,6′-trimethyl-[1,1′:3′,1″-terphenyl]-2-yl)phosphane (Ph*Phine)

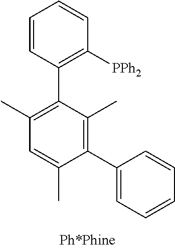

To an oven-dried flask, Mg turnings (105.6 mg, 4.4 mmol, 2.2 equiv.), 1 mL THF and 6 drops of iBu₂AlH were added subsequently. After 5 min of stirring, 3-bromo-2,4,6-trimethyl-1,1′-biphenyl (548 mg, 2 mmol, 1.0 equiv.) was added slowly. The flask was capped under nitrogen and the mixture was heated to 70° C. for 2 h prior to the addition of 2-bromochlorobenze (473.0 mg, 2.4 mmol, 1.2 equiv.). The mixture was stirred at 70° C. for another 3 h. At this time, the reaction mixture was cooled down to room temperature. Then the reaction mixture was transferred to a new oven-dried flask using syringe to remove remaining Mg turnings, and anhydrous CuCl (20 mg, 0.2 mmol, 10 mol %) and LiBr (35 mg, 0.4 mmol, 20%) was added and stirred 15 min. To this solution, ClPPh₂ (485 mg, 2.2 mmol) was added via syringe slowly. The resulting mixture was allowed to reach 70° C. and stirred at this temperature for 16 h. The reaction mixture was cooled down to room temperature and diluted with ethyl acetate. 5 mL 28-30% NH₄OH was added to wash till the organic layer colourless (at least 5 mL*4). The organic layer was washed with brine and dried with MgSO₄, filtered and concentrated to give the crude product; recrystallization in MeOH affords pure white solid (557 mg, 61%), labelled as Ph*Phine. ¹H NMR (600 MHz, Chloroform-d) δ 7.48-7.38 (m, 3H), 7.36-7.23 (m, 13H), 7.22-7.13 (m, 2H), 7.10-6.94 (m, 2H), 2.08 (s, 3H), 1.99 (s, 3H), 1.27 (s, 3H) ppm. ¹³C NMR (151 MHz, Chloroform-d) δ 147.47 (d, J=33.1 Hz), 141.72, 139.51, 138.56, 137.49, 135.10, 134.82, 134.42 (d, J=20.9 Hz), 133.92, 133.53 (d, J=19.3 Hz), 129.83 (d, J=6.2 Hz), 129.60, 129.31, 129.16, 128.74, 128.47, 128.52-127.70 (m), 127.10, 126.30, 21.01, 20.97, 18.79 ppm. ³¹P NMR (243 MHz, CDCl₃) δ=−13.88 ppm.

### Synthesis of Ligand Cy*Phine-Me

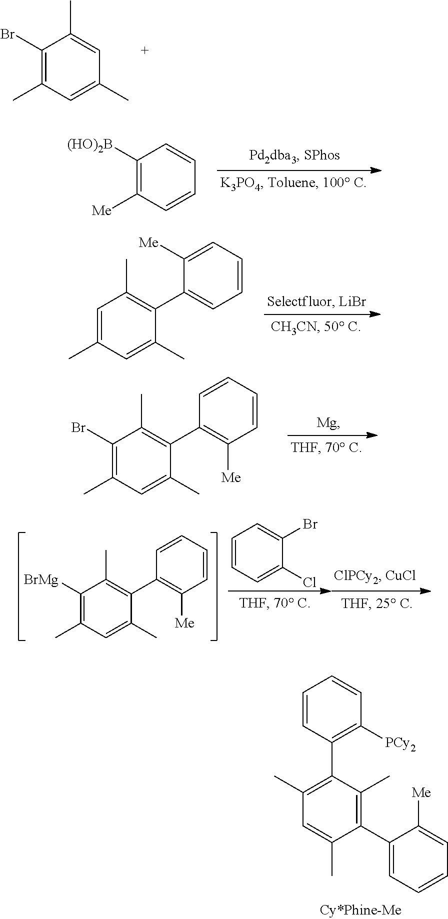

### 2,2′,4,6-tetramethyl-1,1′-biphenyl

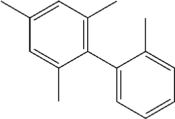 [SMILES: Cc1cc(C)c(-c2ccccc2C)c(C)c1]

2.0 g (10 mmol) of mestlyene bromide, 2.7 g (20 mmol) of o-methyl-phenylbornic acid, 4.224 g (20 mmol) of K₃PO₄, 820 mg (2 mol %) of SPhos and 910 mg (1 mol %) of Pd₂(dba)₃ was added to an oven dried 50 mL flask. The mixture was then vacuumed for 5 min and flush with argon gas. 10 mL of dry THF and 10 mL of dry toluene was added to the flask. The reaction was then heated at 60° C. for 16 hr under argon. The mixture was then cooled to room temperature and filters through silica and washes with Ethyl acetate. The filtrates were collect and remove solvent with vacuum. The crude is purified by column with Hexane as eluent to get colorless oil (6.1 g, 99%). ¹H NMR (600 MHz, Chloroform-d) δ 7.29-7.26 (m, 1H), 7.23 (ddd, J=6.6, 4.1, 1.9 Hz, 2H), 7.01 (dd, J=6.9, 2.0 Hz, 1H), 6.95-6.93 (m, 2H), 2.33 (s, 3H), 1.97 (d, J=0.6 Hz, 3H), 1.91 (s, 6H). ¹³C NMR (151 MHz, Chloroform-d) δ 140.55, 138.18, 136.32, 135.85, 135.71, 129.87, 129.14, 127.97, 126.87, 125.96, 21.06, 20.21, 19.45 ppm.

### 3-bromo-2,2′,4,6-tetramethyl-1,1′-biphenyl

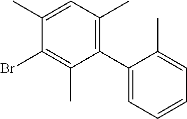 [SMILES: Cc1ccccc1-c1c(C)cc(C)c(Br)c1C]

2.1 g (10 mmol) of 2,2′,4,6-tetramethyl-1,1′-biphenyl, 0.83 g (9.5 mmol) of lithium bromide, 0.9 g (11 mmol) of selectfluor and 30 mL of MeCN was added to a 250 mL flask and stirred 55° C. for 16 hr. The solvent was removed and DCM was added to the crude. The mixture was filtered; the filtrate was wash with sodium thiosulfate. The organic layer was collected and dry with MgSO₄. The crude was then purified with column with Hexane as eluents to get a white solid (2.2 g, 77%). ¹H NMR (600 MHz, Chloroform-d) δ 7.30-7.26 (m, 2H), 7.25-7.22 (m, 1H), 7.03 (q, J=0.8 Hz, 1H), 6.98-6.95 (m, 1H), 2.44 (d, J=0.6 Hz, 3H), 2.06 (s, 3H), 1.96 (s, 3H), 1.86 (d, J=0.6 Hz, 3H). ¹³C NMR (151 MHz, Chloroform-d) δ 140.62, 140.01, 136.70, 135.80, 135.71, 134.60, 130.00, 129.49, 128.97, 127.24, 126.10, 125.33, 23.96, 21.28, 20.09, 19.48 ppm.

### Dicyclohexyl(2′,2″,4′,6′-tetramethyl-[1,1′:3′,1″-terphenyl]-2-yl)phos-phane (Cy*Phine-Me)

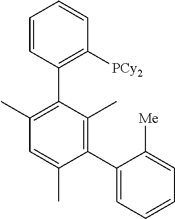

To an oven-dried flask Mg turnings (105.6 mg, 4.4 mmol, 2.2 equiv.), 1 mL THF and 6 drops of iBu₂AlH were added subsequently. After 5 min of stirring, 3-bromo-2,2′,4,6-tetramethyl-1,1′-biphenyl (578 mg, 2 mmol, 1.0 equiv.) was added slowly. The mixture was heated to 70° C. for 2 h prior to the addition of 2-bromochlorobenze (473.0 mg, 2.4 mmol). The mixture was stirred at 70° C. for another 3 h. At this time, the reaction mixture was cooled down to room temperature. Then the reaction mixture was transferred to a new oven-dried flask using syringe to remove remaining Mg turnings, and anhydrous CuCl (10 mg, 0.1 mmol, 5 mol %) was added and stirred 15 min. To this solution, ClPCy₂ (510 mg, 2.2 mmol, 1.1 equiv) was added via syringe slowly. The resulting mixture was stirred at room temperature for 23 h. The reaction mixture was cooled down to room temperature and diluted with ethyl acetate. 5 mL 28-30% NH₄OH was added to wash till the organic layer colourless (at least 10 mL×4). The organic layer was washed with brine and dried with MgSO₄, filtered and concentrated to give the crude product; recrystallization in MeOH affords pure white solid (289 mg, 30%), labelled as Cy*Phine-Me. ¹H NMR (600 MHz, Chloroform-d) δ 7.58-7.53 (m, 1H), 7.39-7.35 (m, 1H), 7.33 (td, J=7.4, 1.6 Hz, 1H), 7.29-7.27 (m, 1H), 7.25-7.22 (m, 2H), 7.12 (dddd, J=16.5, 7.5, 3.8, 1.6 Hz, 1H), 7.09-7.06 (m, 1H), 7.05 (s, 1H), 7.02 (s, OH), 6.99-6.96 (m, 1H), 2.04 (d, J=5.5 Hz, 6H), 1.94 (d, J=8.5 Hz, 4H), 1.78 (t, J=9.9 Hz, 2H), 1.74-1.62 (m, 7H), 1.60-1.54 (m, 6H), 1.34-1.20 (m, 5H), 1.09 (ddt, J=17.0, 12.3, 9.5 Hz, 7H) ppm. ¹³C NMR (151 MHz, Chloroform-d) δ 148.52, 141.50, 141.19, 138.68, 138.63, 136.05, 135.83, 134.56, 134.49, 134.31, 134.17, 133.68, 133.38, 132.56, 132.47, 130.67, 130.63, 129.88, 129.81, 129.18, 129.12, 128.55, 128.44, 128.40, 128.33, 126.73, 126.66, 126.06, 125.92, 34.83, 34.79, 34.73, 34.69, 33.18, 33.08, 32.72, 32.63, 30.84, 30.76, 30.72, 30.64, 30.28, 30.19, 30.16, 30.07, 29.98, 29.87, 29.84, 29.73, 29.27, 29.20, 29.09, 29.02, 27.78, 27.75, 27.68, 27.62, 27.55, 27.49, 27.36, 27.32, 27.29, 27.25, 27.18, 26.51, 26.48, 26.40, 26.38, 21.51, 21.46, 20.39, 20.35, 19.52, 19.50, 19.49, 19.38, 19.26 ppm. ³¹P {¹H} (243 MHz, Chloroform-d) δ −8.76 ppm.

### Synthesis of Ligand tBu*Phine-Me

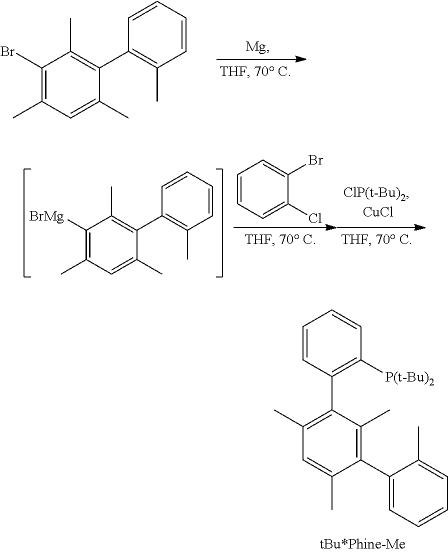

### Di-tert-butyl(2′,4′,6′-trimethyl-[1,1′:3′,1″-terphenyl]-2-yl)phosphane (tBu*Phine-Me)

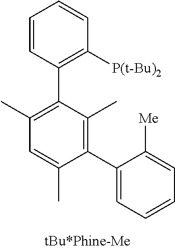

To an oven-dried flask Mg turnings (105.6 mg, 4.4 mmol, 2.2 equiv.), 1 mL THF and 6 drops of iBu₂AlH were added subsequently. After 5 min of stirring, 3-bromo-2,2′,4,6-tetramethyl-1,1′-biphenyl (578 mg, 2 mmol, 1.0 equiv.) was added slowly. The mixture was heated to 70° C. for 2 h prior to the addition of 2-bromochlorobenze (473.0 mg, 2.4 mmol). The mixture was stirred at 70° C. for another 3 h. At this time, the reaction mixture was cooled down to room temperature. Then the reaction mixture was transferred to a new oven-dried flask using syringe to remove remaining Mg turnings, and anhydrous CuCl (200 mg, 2.0 mmol, 1.0 equiv.) was added and stirred 15 min. To this solution, ClP^{t}Bu₂ (398 mg, 2.2 mmol, 1.1 equiv.) was added via syringe slowly. The resulting mixture was allowed to reach 70° C. and stirred at this temperature for 16 h. The reaction mixture was cooled down to room temperature and diluted with ethyl acetate. 5 mL 28-30% NH₄OH was added to wash till the organic layer colourless (at least 10 mL×4). The organic layer was washed with brine and dried with MgSO₄, filtered and concentrated to give the crude product; recrystallization in MeOH affords pure white solid (130 mg, 15%), labelled as tBu*Phine-Me. ¹H NMR (600 MHz, Chloroform-d) δ 7.85 (t, J=6.2 Hz, 1H), 7.40-7.34 (m, 1H), 7.31 (t, J=7.6 Hz, 1H), 7.28 (dd, J=6.1, 3.5 Hz, 1H), 7.23 (ddd, J=12.5, 5.5, 2.4 Hz, 2H), 7.19-7.10 (m, 2H), 7.10-6.93 (m, 3H), 2.13 (d, J=23.5 Hz, 3H), 2.04 (d, J=3.8 Hz, 3H), 1.94 (d, J=10.9 Hz, 4H), 1.89 (d, J=15.5 Hz, 1H), 1.61 (d, J=9.2 Hz, 2H), 1.54 (s, 2H), 1.24 (d, J=11.4 Hz, 5H), 1.20-1.08 (m, 13H) ppm. ¹³C NMR (151 MHz, Chloroform-d) δ 141.64, 141.27, 139.82, 139.78, 138.62, 138.56, 136.90, 136.26, 136.14, 136.08, 135.78, 134.63, 134.24, 134.13, 134.03, 133.72, 131.58, 131.54, 129.91, 129.79, 129.18, 129.12, 129.08, 128.52, 128.47, 128.37, 128.35, 128.31, 126.72, 126.69, 126.60, 126.07, 125.88, 125.44, 125.38, 76.84, 32.76, 32.73, 32.68, 32.63, 32.60, 32.56, 32.52, 32.46, 31.52, 31.42, 31.03, 30.99, 30.93, 30.89, 30.58, 30.48, 22.20, 22.15, 22.13, 22.10, 20.66, 20.63, 20.30, 20.27, 20.07, 19.56, 19.49 ppm. ³¹P {¹H} (243 MHz, Chloroform-d) δ 24.1 ppm.

### Synthesis of Ligand OMe-tBu*Phine

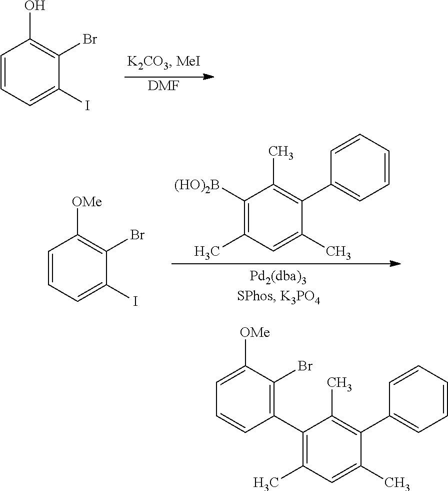

### 2-bromo-1-iodo-3-methoxybenzene

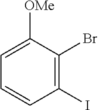

An oven-dried 10 mL round bottom flask which was equipped with a magnetic stir bar and fitted with a septum and argon filled balloon was charged with 2-bromo-3-iodophenol (150 mg, 0.5 mmol) and anhydrous DMF (0.3 mL). To the reaction mixture, K₂CO₃ (173 mg, 1.25 mmol) was added and the reaction mixture was stirred at room temperature for 30 min before adding MeI (0.16 mL, 2.5 mmol). The reaction mixture was allowed to stir at room temperature for 14 h and water (10 mL) was added. The mixture was extracted with diethyl ether (3×20 mL) and the combined organic extracts were washed with brine, dried over Na₂SO₄, filtered and the solvent was removed with the aid of a rotary evaporator. The crude material was purified by flash chromatography (silica gel, 2:98 EtOAc:hexanes) to afford an off-white colored solid (0.138 g, 88% yield). ¹H NMR (600 MHz, CDCl₃) δ 7.48-7.46 (m, 1H), 6.99 (t, ³J_{H,H}=8.1 Hz, 1H), 6.85-6.83 (m, 1H), 3.86 (s, 3H) ppm.

### 2-bromo-3-methoxy-2′,4′,6′-trimethyl-1,1′:3′,1″-terphenyl

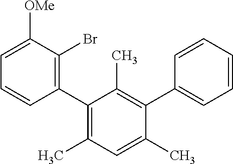 [SMILES: COc1cccc(-c2c(C)cc(C)c(-c3ccccc3)c2C)c1Br]

An oven-dried 10 mL microwave reaction vial which was equipped with a magnetic stir bar was sequentially charged with Pd₂(dba)₃ (2.9 mg, 0.003 mmol), SPhos (2.9 mg, 0.007 mmol), K₃PO₄ (203 mg, 0.96 mmol), (2,4,6-trimethyl-[1,1′-biphenyl]-3-yl)boronic acid (115 mg, 0.48 mmol), 2-bromo-1-iodo-3-methoxybenzene (100 mg, 0.32 mmol) and anhydrous toluene (0.64 mL) inside a glove-box. The vial was sealed and the reaction mixture was allowed to stir at 110° C. for 16 h. The reaction mixture was cooled and water (20 mL) was added. The mixture was extracted with EtOAc (3×25 mL) and the combined organic extracts were washed with brine, dried over Na₂SO₄, filtered and the solvent was removed with the aid of a rotary evaporator. The crude material was purified by gravity column chromatography (silica gel, 2:98 EtOAc:hexanes) to afford an off-white colored solid (0.106 g, 87% yield). H NMR (600 MHz, CDCl₃) δ 7.41-7.38 (m, 2H), 7.38-7.28 (m, 2H), 7.18-7.14 (m, 2H), 7.03 (s, 1H), 6.86 (dd, ³J_{H,H}=8.4 Hz, ⁴J_{H,H}=1.2 Hz, 1H), 6.79 (dd, ³J_{H,H}=7.8 Hz, ⁴J_{H,H}=1.8 Hz, 1H), 3.93 (s, 3H), 2.03 (s, 3H), 1.97 (s, 3H), 1.63 (s, 3H).

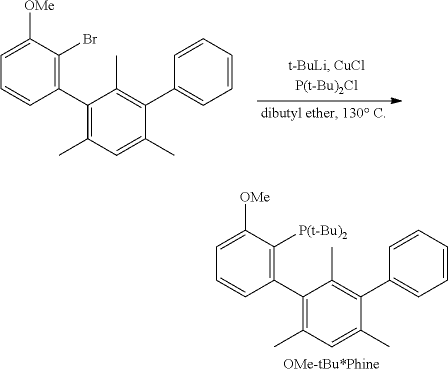

### Di-tert-butyl(3-methoxy-2′,4′,6′-trimethyl-[1,1′:3′,1″-terphenyl]-2-yl)phosphane (OMe-tBu*Phine)

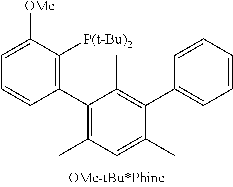

An oven-dried 10 mL microwave reaction vial which was equipped with a magnetic stir bar was charged with 2-bromo-3-methoxy-2′,4′,6′-trimethyl-1,1′:3′,1″-terphenyl (40 mg, 0.1 mmol) and dibutyl ether (0.2 mL) inside a glove-box. The vial was sealed and brought out of the glove-box. The reaction mixture was cooled to −78° C. and t-BuLi (1.7 M in pentane, 0.13 mL, 0.22 mmol) was added dropwise in 5 min. It was allowed to stir at this temperature for 1 h after which the vial was again transferred to a glove-box and CuCl (11 mg, 0.11 mmol) was added to it. The above was stirred for 10 min before adding di-tert-butyl chlorophosphine (0.021 mL, 0.11 mmol) slowly. The vial was brought out of the glove-box and the reaction mixture was allowed to stir at 130° C. for 10 h. The reaction mixture was cooled and EtOAc (10 mL) and 30% NH₄OH (10 mL) were added. The two layers were shaken in a separatory funnel and separated. The organic phase was washed with 30% NH₄OH until the aqueous phase was no longer blue. The combined aqueous washes were back-extracted with EtOAc (20 mL) and the combined organic extracts were washed with brine, dried over Na₂SO₄, filtered and the solvent was removed with the aid of a rotary evaporator. The crude material was purified by trituration in degassed MeOH (0.2 mL) at −20° C. to afford an off-white colored solid (0.0267 g, 57% yield). ¹H NMR (600 MHz, CDCl₃) δ 7.39-7.35 (m, 2H), 7.32-7.28 (m, 2H), 7.17-7.15 (m, 1H), 7.17-7.15 (m, 1H), 6.95 (s, 1H), 6.81 (d, ³J_{H,H}=7.8 Hz, 1H), 6.75-6.72 (m, 1H), 3.79 (s, 3H), 2.08 (s, 3H), 1.99 (s, 3H), 1.62 (s, 3H), 1.15 (d, ³J_{H,P}=12.0 Hz, 9H), 1.09 (d, ³J_{H,P}=12.0 Hz, 9H); ¹³C NMR (150 MHz, CDCl₃) δ 162.19, 142.50, 139.38, 134.72, 134.16, 130.19, 129.68, 129.48, 128.69, 128.49, 128.35, 126.40, 124.54, 124.49, 108.65, 54.04, 34.02, 33.84, 33.62, 33.44, 31.84, 31.73, 31.48, 31.37, 22.34, 21.25, 21.03, 1.12; ³¹P NMR (161 MHz, CDCl₃) δ 35.58.

### Synthesis of Ligand OiPr-tBu*Phine

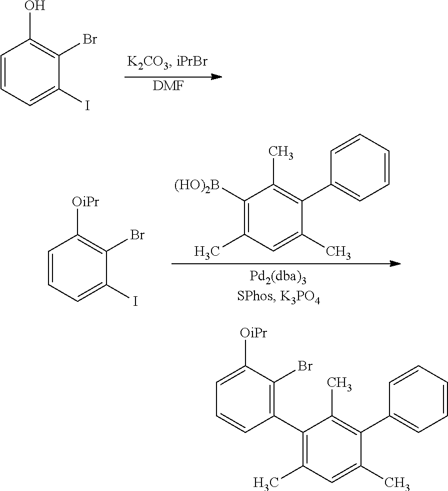

### 2-bromo-1-iodo-3-isopropoxybenzene

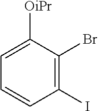 [SMILES: CC(C)Oc1cccc(I)c1Br]

An oven-dried 10 mL round bottom flask which was equipped with a magnetic stir bar and fitted with a reflux condenser was charged with 2-bromo-3-iodophenol (200 mg, 0.67 mmol), anhydrous DMF (0.5 mL), K₂CO₃ (231 mg, 1.67 mmol) and 2-bromopropane (0.16 mL, 1.67 mmol) in succession. The reaction mixture was allowed to stir at 70° C. for 14 h and water (10 mL) was added. The mixture was extracted with diethyl ether (3×20 mL) and the combined organic extracts were washed with brine, dried over Na₂SO₄, filtered and the solvent was removed with the aid of a rotary evaporator. The crude material was purified by flash chromatography (silica gel, 2:98 EtOAc:hexanes) to afford an oil (0.173 g, 76% yield). ¹H NMR (600 MHz, CDCl₃) δ 7.46-7.44 (m, 1H), 6.94 (t, ³J_{H,H}=7.8 Hz, 1H), 6.85-6.83 (m, 1H), 4.52 (septet, ³J_{H,H}=6.0 Hz, 1H), 1.36 (d, ³J_{H,H}=6.0 Hz, 6H) ppm. 2-bromo-3-isopropoxy-2′,4′,6′-trimethyl-1,1′:3′,1″-terphenyl (OiPr-Mesityl-Br).

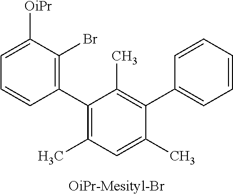 [SMILES: Cc1cc(C)c(-c2cccc(OC(C)C)c2Br)c(C)c1-c1ccccc1]

An oven-dried 10 mL microwave reaction vial which was equipped with a magnetic stir bar was sequentially charged with Pd₂(dba)₃ (4.3 mg, 0.005 mmol), SPhos (4.2 mg, 0.01 mmol), K₃PO₄ (299 mg, 1.41 mmol), (2,4,6-trimethyl-[1,1′-biphenyl]-3-yl)boronic acid (169 mg, 0.7 mmol), 2-bromo-1-iodo-3-isopropoxybenzene (0.16 g, 0.47 mmol) and anhydrous toluene (0.94 mL) inside a glove-box. The vial was sealed and the reaction mixture was allowed to stir at 110° C. for 16 h. The reaction mixture was cooled and water (20 mL) was added. The mixture was extracted with EtOAc (3×25 mL) and the combined organic extracts were washed with brine, dried over Na₂SO₄, filtered and the solvent was removed with the aid of a rotary evaporator. The crude material was purified by flash chromatography (silica gel, 2:98 EtOAc:hexanes) to afford a gummy mass (0.157 g, 82% yield). ¹H NMR (600 MHz, CDCl₃) δ 7.41-7.37 (m, 2H), 7.31-7.25 (m, 2H), 7.18-7.14 (m, 2H), 7.03 (s, 1H), 6.88 (dd, ³J_{H,H}=8.4 Hz, ⁴J_{H,H}=1.2 Hz, 1H), 6.76 (dd, ³J_{H,H}=7.8 Hz, ⁴J_{H,H}=1.2 Hz, 1H), 4.58 (septet, ³J_{H,H}=6.6 Hz, 1H), 2.03 (s, 3H), 1.98 (s, 3H), 1.58 (s, 3H), 1.41-1.39 (m, 6H).

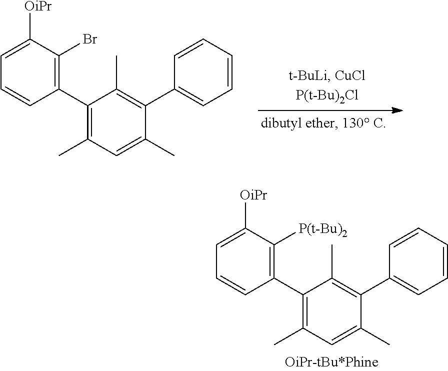

### Di-tert-butyl(3-isopropoxy-2′,4′,6′-trimethyl-[1,1′:3′,1″-terphenyl]-2-yl)phosphane (OiPr-tBu*Phine)

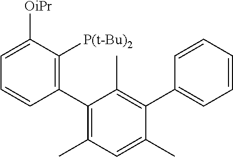

An oven-dried 10 mL microwave reaction vial which was equipped with a magnetic stir bar was charged with 2-bromo-3-isopropoxy-2′,4′,6′-trimethyl-1,1′:3′,1″-terphenyl (138 mg, 0.33 mmol) and dibutyl ether (0.35 mL) inside a glove-box. The vial was sealed and brought out of the glove-box. The reaction mixture was cooled to −78° C. and t-BuLi (1.7 M in pentane, 0.4 mL, 0.69 mmol) was added dropwise in 5 min. It was allowed to stir at this temperature for 1 h after which the vial was again transferred to a glove-box and CuCl (36.7 mg, 0.37 mmol) was added to it. The above was stirred for 10 min before adding di-tert-butyl chlorophosphine (0.07 mL, 0.37 mmol) slowly. The vial was brought out of the glove-box and the reaction mixture was allowed to stir at 130° C. for 14 h. The reaction mixture was cooled and EtOAc (20 mL) and 30% NH₄OH (20 mL) were added. The two layers were shaken in a separatory funnel and separated. The organic phase was washed with 30% NH₄OH until the aqueous phase was no longer blue. The combined aqueous washes were back-extracted with EtOAc (20 mL) and the combined organic extracts were washed with brine, dried over Na₂SO₄, filtered and the solvent was removed with the aid of a rotary evaporator. The crude material was purified by trituration in degassed MeOH (0.5 mL) at −20° C. to afford an off-white solid (0.101 g, 63% yield). ¹H NMR (600 MHz, CDCl₃) δ 7.41-7.36 (m, 2H), 7.28-7.27 (m, 2H), 7.16-7.15 (m, 1H), 7.08-7.06 (m, 1H), 6.94 (s, 1H), 6.81 (d, ³J_{H,H}=7.8 Hz, 1H), 6.69-6.64 (m, 1H), 4.74 (m, 1H), 2.07 (s, 3H), 1.99 (s, 3H), 1.61 (s, 3H), 1.57 (s, 3H), 1.45-1.43 (m, 6H), 1.20 (d, ³J_{H,P}=12.0 Hz, 9H), 1.14 (d, ³J_{H,P}=12.0 Hz, 9H); ¹³C NMR (150 MHz, CDCl₃) δ 160.79, 160.76, 134.73, 134.72, 134.23, 134.22, 129.62, 129.44, 129.23, 128.68, 128.48, 128.42, 128.29, 126.40, 66.05, 31.86, 31.60, 22.30, 22.23, 22.09, 21.22, 20.98, 15.47, 1.22; ³¹P NMR (161 MHz, CDCl₃) δ 35.34.

### Synthesis of Ligand OCy-tBu*Phine

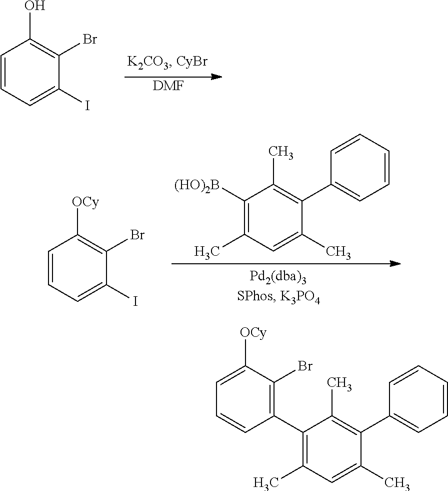

### 2-bromo-1-(cyclohexyloxy)-3-iodobenzene

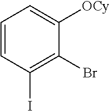

An oven-dried 10 mL round bottom flask which was equipped with a magnetic stir bar and fitted with a reflux condenser was charged with 2-bromo-3-iodophenol (400 mg, 1.34 mmol), anhydrous DMF (2.0 mL), K₂CO₃ (924 mg, 6.69 mmol) and bromocyclohexane (1.65 mL, 13.4 mmol) in succession. The reaction mixture was allowed to stir at 110° C. for 16 h and water (20 mL) was added. The mixture was extracted with diethyl ether (3×20 mL) and the combined organic extracts were washed with brine, dried over Na₂SO₄, filtered and the solvent was removed with the aid of a rotary evaporator. The crude material was purified by flash chromatography (silica gel, 2:98 EtOAc:hexanes) to afford an oil (0.23 g, 45% yield). H NMR (600 MHz, CDCl₃) δ 7.44-7.43 (m, 1H), 6.93 (t, ³J_{H,H}=7.8 Hz, 1H), 6.85-6.83 (m, 1H), 4.29 (septet, ³J_{H,H}=3.6 Hz, 1H), 1.90-1.87 (m, 2H), 1.82-1.73 (m, 2H), 1.67-1.62 (m, 2H), 1.40-1.31 (m, 4H).

### 2-bromo-3-(cyclohexyloxy)-2′,4′,6′-trimethyl-1,1′:3′,1″-terphenyl (OCy-Mesityl-Br)

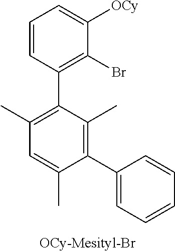

An oven-dried 10 mL microwave reaction vial which was equipped with a magnetic stir bar was sequentially charged with Pd₂(dba)₃ (3.5 mg, 0.004 mmol), SPhos (3.5 mg, 0.008 mmol), K₃PO₄ (242.3 mg, 1.14 mmol), (2,4,6-trimethyl-[1,1′-biphenyl]-3-yl)boronic acid (119 mg, 0.49 mmol), 2-bromo-1-(cyclohexyloxy)-3-iodobenzene (0.145 g, 0.38 mmol) and anhydrous toluene (0.76 mL) inside a glove-box. The vial was sealed and the reaction mixture was allowed to stir at 110° C. for 16 h. The reaction mixture was cooled and water (20 mL) was added. The mixture was extracted with EtOAc (3×25 mL) and the combined organic extracts were washed with brine, dried over Na₂SO₄, filtered and the solvent was removed with the aid of a rotary evaporator. The crude material was purified by flash chromatography (silica gel, 2:98 EtOAc:hexanes) to afford a gummy mass (0.13 g, 76% yield). ¹H NMR (600 MHz, CDCl₃) δ 7.41-7.37 (m, 2H), 7.31-7.25 (m, 2H), 7.18-7.14 (m, 2H), 7.03 (s, 1H), 6.89 (dd, ³J_{H,H}=8.4 Hz, ⁴J_{H,H}=1.2 Hz, 1H), 6.75 (dd, ³J_{H,H}=7.2 Hz, ⁴J_{H,H}=1.2 Hz, 1H), 4.33 (septet, ³J_{H,H}=3.6 Hz, 1H), 2.03 (s, 3H), 2.00-1.94 (m, 5H), 1.70-1.66 (m, 2H), 1.64-1.60 (m, 2H), 1.58 (s, 3H), 1.39-1.34 (m, 4H).

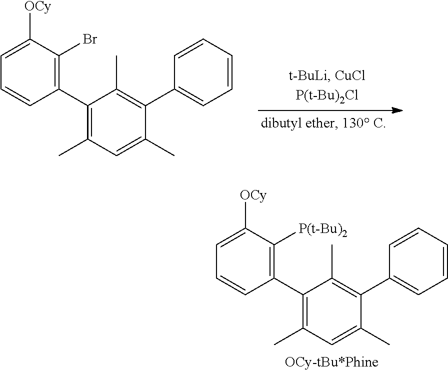

### Di-tert-butyl(3-(cyclohexyloxy)-2′,4′,6′-trimethyl-[1,1′:3′,1″-ter-phenyl]-2-yl)phosphane (OCy-tBu*Phine)

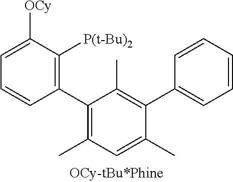

An oven-dried 10 mL microwave reaction vial which was equipped with a magnetic stir bar was charged with 2-bromo-3-(cyclohexyloxy)-2′,4′,6′-trimethyl-1,1′:3′,1″-terphenyl (129 mg, 0.29 mmol) and dibutyl ether (0.3 mL) inside a glove-box. The vial was sealed and brought out of the glove-box. The reaction mixture was cooled to −78° C. and t-BuLi (1.7 M in pentane, 0.35 mL, 0.59 mmol) was added dropwise in 5 min. It was allowed to stir at this temperature for 1 h after which the vial was again transferred to a glove-box and CuCl (31.3 mg, 0.32 mmol) was added to it. The above was stirred for 10 min before adding di-tert-butyl chlorophosphine (0.06 mL, 0.32 mmol) slowly. The vial was brought out of the glove-box and the reaction mixture was allowed to stir at 130° C. for 12 h. The reaction mixture was cooled and EtOAc (20 mL) and 30% NH₄OH (20 mL) were added. The two layers were shaken in a separatory funnel and separated. The organic phase was washed with 30% NH₄OH until the aqueous phase was no longer blue. The combined aqueous washes were back-extracted with EtOAc (20 mL) and the combined organic extracts were washed with brine, dried over Na₂SO₄, filtered and the solvent was removed with the aid of a rotary evaporator. The crude material was purified by trituration in degassed MeOH (0.5 mL) at −20° C. to afford an off-white solid (0.0916 g, 62% yield). ¹H NMR (600 MHz, CDCl₃) δ 7.38-7.36 (m, 2H), 7.28-7.25 (m, 2H), 7.17-7.15 (m, 1H), 7.09-7.07 (m, 1H), 6.93 (s, 1H), 6.81 (d, ³J_{H,H}=8.4 Hz, 1H), 6.67-6.65 (m, 1H), 4.33 (septet, ³J_{H,H}=3.6 Hz, 1H), 2.27-2.21 (m, 2H), 2.07 (s, 3H), 1.99 (s, 3H), 1.87-1.83 (m, 2H), 1.70-1.68 (m, 1H), 1.60 (s, 3H), 1.58-1.56 (m, 1H), 1.52-1.50 (m, 1H), 1.42-1.35 (m, 3H), 1.22 (d, ³J_{H,P}=12.0 Hz, 9H), 1.16 (d, ³J_{H,P}=12.0 Hz, 9H); ¹³C NMR (150 MHz, CDCl₃) δ 160.76, 160.74, 153.23, 142.57, 139.24, 134.29, 133.94, 130.02, 129.70, 129.50, 128.67, 128.47, 128.20, 126.37, 123.67, 123.62, 109.60, 76.62, 33.77, 33.58, 33.32, 33.13, 32.59, 32.49, 32.13, 32.02, 31.87, 31.76, 25.90, 25.42, 25.37, 22.40, 22.36, 21.25, 21.01; ³¹P NMR (161 MHz, CDCl₃) δ 35.43 ppm.

### TIP Series

### Synthesis of Ligand TIP-Cy*Phine

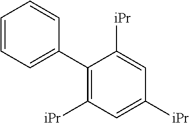 [SMILES: CC(C)c1cc(C(C)C)c(-c2ccccc2)c(C(C)C)c1]

### 2,4,6-triisopropyl-1,1′-biphenyl

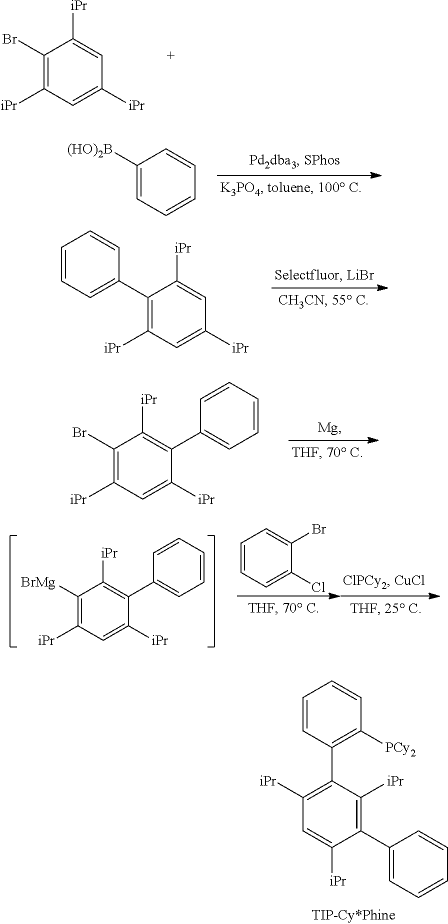

To a 50 mL oven-dried flask was added 2-bromo-1,3,5-triisopropylbenzene (10.14 g, 0.036 mol, 1.0 equiv.), phenyl boronic acid (7.32 g, 0.06 mol, 1.6 equiv.), K₃PO₄ (17.0 g, 0.80 mol, 2.2 equiv), Pd₂(dba)₃ (0.366 g, 0.4 mmol) and SPhos (0.328 g, 0.8 mmol). The flask was evacuated and back-filled with argon three times prior to 40 mL of dry THF and 40 mL of dry toluene was added via syringe. The mixture was then stirred and heated in a 100° C. oil bath for 16 hr. Upon reaction completion, as determined by GC-MS, the mixture was filtered through silica and washed with EtOAc. The filtrate was collected and the solvent was removed under vacuum. The crude residue was purified by flash chromatography on silica gel using an EtOAc/hexanes mixture (0 to 10% EtOAc) as the eluent to get a white solid (9 g, 90%). ¹H NMR (600 MHz, Chloroform-d) δ 7.42 (dd, J=8.1, 6.8 Hz, 2H), 7.39-7.34 (m, 1H), 7.23-7.19 (m, 2H), 7.09 (s, 1H), 2.97 (p, J=7.0 Hz, 1H), 2.62 (ddd, J=12.7, 7.7, 6.4 Hz, 2H), 1.34 (dd, J=7.0, 0.9 Hz, 5H), 1.11 (dd, J=6.9, 0.9 Hz, 11H) ppm. ¹³C NMR (151 MHz, CDCl₃) δ 147.82, 146.51, 140.84, 137.08, 129.80, 127.91, 126.40, 120.53, 77.26, 77.05, 76.84, 34.28, 30.28, 24.25, 24.13 ppm.

### 3-bromo-2,4,6-triisopropyl-1,1′-biphenyl

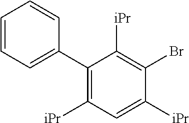 [SMILES: CC(C)c1cc(C(C)C)c(-c2ccccc2)c(C(C)C)c1Br]

To a 250 mL oven-dried flask was added 2,4,6-triisopropyl-1,1′-biphenyl (10.0 g, 0.036 mol, 1.0 equiv.), Selectfluor (14.5 g, 0.041 mol), LiBr (3.6 g, 0.041 mol). The flask was evacuated and back-filled with argon three times prior to 180 mL of anhydrous acetonitrile was added via syringe. The mixture was then stirred at room temperature overnight. Upon reaction completion, the solvent was removed and DCM was added to the crude. The mixture was filtered; the filtrate was wash with sodium thiosulfate. The organic layer was collected and dry with MgSO₄. The crude was then purified with column with Hexane as eluents as the eluent to get a white solid (11.8 g, 91.5%). ¹H NMR (600 MHz, Chloroform-d) δ 7.44-7.35 (m, 3H), 7.16 (s, 2H), 3.62 (p, J=6.8 Hz, 1H), 3.08-2.88 (m, 1H), 2.49 (p, J=6.9 Hz, 1H), 1.32 (d, J=6.9 Hz, 12H), 1.07 (d, J=6.9 Hz, 6H) ppm.

### Dicyclohexyl(2′,4′,6′-triisopropyl-[1,1′:3′,1″-terphenyl]-2-yl)phos-phane (TIP-Cy*Phine)

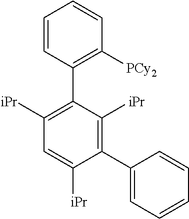

To an oven-dried flask fitted with a septum was sequentially added activated Mg with DIBAL (0.29 g, 12.2 mmol, 2.2 equiv), 12 mL of THF and 3-bromo-2,4,6-triisopropyl-1,1′-biphenyl (2 g, 5.6 mmol, 1.0 equiv). The mixture was then heated to 70° C. in an oil bath and stirred for 2 h prior to the dropwise addition of 2-bromochlorobenzene (1.2 g, 6.2 mmol, 1.1 equiv) at 70° C., after which the mixture was stirred for another 3 h. At this time, the mixture was cooled to room temperature and was transferred to another oven-dried flask with charge of CuCl (27.7 mg, 0.28 mmol, 5 mol %). Additional of 4 mL anhydrous THF was rinsed and also was transferred. PCy₂Cl (1.43 g, 6.2 mmol, 1.1 equiv) was subsequently added at room temperature and stirred overnight (16 h). After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (2.6 g, 84%), labelled as TIP-Cy*Phine. ¹H NMR (600 MHz, CDCl₃) δ=7.39-7.29 (m, 5H), 7.30-7.19 (m, 4H), 7.15 (s, 1H), 2.61 (sep, J=7.3 Hz, 1H), 2.45 (sep, J=6.5 Hz, 1H), 2.34 (sep, J=6.9 Hz, 1H), 1.92 (m, 3H), 1.84-1.50 (m, 10H), 1.24 (m, 12H), 1.11 (d, J=6.8 Hz, 3H), 1.03 (d, J=6.8 Hz, 3H), 0.97 (d, J=6.7 Hz, 3H), 0.89 (d, J=6.9 Hz, 3H), 0.53 (d, J=7.2 Hz, 3H) ppm. ¹³C NMR (150 MHz, CDCl₃) δ=149.00 (d, J_{CP}=30.5 Hz), 142.42, 139.92, 139.72 (d, J_{CP}=5.9 Hz), 135.92 (d, J_{CP}=18.8 Hz), 135.04, 134.96 (d, J_{CP}=1.9 Hz), 134.17, 132.93 (d, J_{CP}=3.2 Hz), 130.96 (d, J_{CP}=5.9 Hz), 129.69 (d, J_{CP}=16.0 Hz), 128.90, 128.72, 126.58 (d, J_{CP}=26.4 Hz), 35.12 (d, J_{CP}=15.4 Hz), 33.25 (d, J_{CP}=13.8 Hz), 31.04 (d, J_{CP}=12.0 Hz), 30.43 (dd, J_{CP}=27.1, 14.9 Hz), 29.55 (d, J_{CP}=10.9 Hz), 28.09 (dd, J_{CP}=14.6, 9.5 Hz), 27.65 (d, J_{CP}=10.1 Hz), 26.86 (d, J_{CP}=17.5 Hz), 21.85 (d, J_{CP}=6.2 Hz), 21.31, 20.37 ppm. ³¹P NMR (243 MHz, CDCl₃) δ=−11.78 ppm.

### Synthesis of Ligand TIP-tBu*Phine

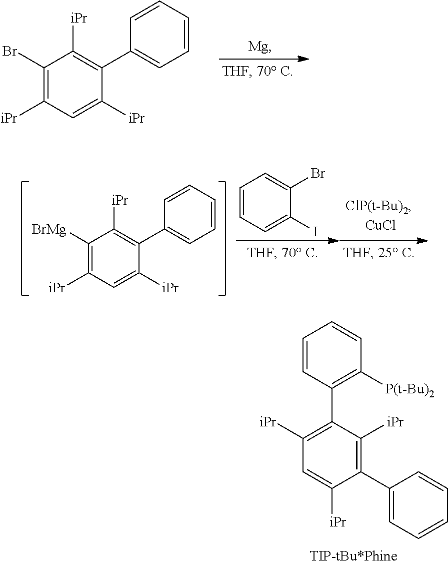

### Di-tert-butyl(2′,4′,6′-triisopropyl-[1,1′:3′,1″-terphenyl]-2-yl) phosphane (TIP-tBu*Phine)

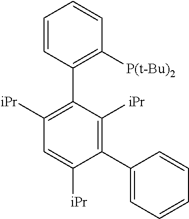

To an oven-dried flask fitted with a septum was sequentially added activated Mg with DIBAL (52.8 mg, 2.2 mmol, 2.2 equiv), 1 mL of THF and 3-bromo-2,4,6-triisopropyl-1,1′-biphenyl (360 mg, 1 mmol, 1.0 equiv). The mixture was then heated to 70° C. in an oil bath and stirred for 2 h prior to the dropwise addition of 2-bromochlorobenzene (237 mg, 1.2 mmol, 1.2 equiv) at 70° C., after which was stirred for another 3 h. At this time, the mixture was cooled to room temperature and was transferred to another oven-dried flask with charge of CuCl (99 mg, 1 mmol, 1.0 equiv). Additional of 1 mL anhydrous THF was rinsed and also was transferred. P(t-Bu)₂Cl (199 mg, 1.1 mmol, 1.1 equiv) was subsequently added at 70° C. and stirred 24 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (175 mg, 36%), labelled as TIP-tBu*Phine. ¹H NMR (600 MHz, Benzene-d₆) δ 8.06 (d, J=7.6 Hz, 1H), 7.65 (s, 2H), 7.64-7.58 (m, 3H), 7.53 (d, J=7.5 Hz, 1H), 7.41-7.27 (m, 5H), 3.40 (p, J=7.1 Hz, 1H), 2.90 (dp, J=31.5, 6.8 Hz, 2H), 1.60 (d, J=6.8 Hz, 3H), 1.45-1.27 (m, 30H), 1.05 (d, J=7.2 Hz, 3H) ppm. ¹³C NMR (151 MHz, Benzene-d₆) δ 147.48, 146.56, 142.67, 142.10, 137.10, 136.28, 132.66 (d, J_{CP}=7.1 Hz), 132.29, 130.89, 127.19 (d, J_{CP}=12.1 Hz), 126.59, 125.54, 119.45, 33.33-32.15 (m), 31.26 (d, J_{CP}=24.6 Hz), 31.08, 30.79 (d, J_{CP}=14.9 Hz), 29.65, 26.79, 24.73, 24.53, 24.21, 23.57, 22.95 ppm. ³¹P NMR (243 MHz, CDCl₃) δ=21.88 ppm.

### Synthesis of Ligand TIP-Ph*Phine

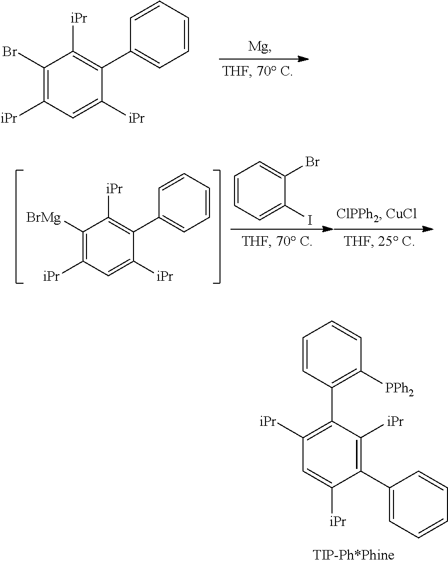

### Diphenyl(2′,4′,6′-triisopropyl-[1,1′:3′,1″-terphenyl]-2-yl)phos phane (TIP-Ph*Phine)

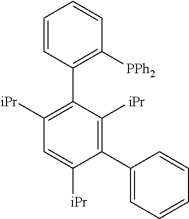

To an oven-dried flask fitted with a septum was sequentially added activated Mg with DIBAL (52.8 mg, 2.2 mmol, 2.2 equiv), 1 mL of THF and 3-bromo-2,4,6-triisopropyl-1,1′-biphenyl (360 mg, 1 mmol, 1.0 equiv). The mixture was then heated to 70° C. in an oil bath and stirred for 2 h prior to the dropwise addition of 2-bromochlorobenzene (237 mg, 1.2 mmol, 1.2 equiv) at 70° C., after which was stirred for another 3 h. At this time, the mixture was cooled to room temperature and was transferred to another oven-dried flask with charge of CuCl (9.9 mg, 0.1 mmol, 10 mol %) and LiBr (17.4 mg, 0.2 mmol, 20 mol %). Additional of 1 mL anhydrous THF was rinsed and also was transferred. PPh₂Cl (242 mg, 1.1 mmol, 1.1 equiv) was subsequently and the resulting mixture was stirred 24 h at room temperature. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (221 mg, 41%), labelled as TIP-Ph*Phine. ¹H NMR (600 MHz, Chloroform-d) δ 7.40 (d, J=7.4 Hz, 1H), 7.36-7.30 (m, 7H), 7.28-7.26 (m, 4H), 7.24-7.18 (m, 7H), 7.13 (d, J=2.2 Hz, 1H), 2.67 (tt, J=7.1, 3.5 Hz, 1H), 2.49 (pd, J=6.9, 2.0 Hz, 1H), 2.32 (tt, J=6.9, 3.4 Hz, 1H), 1.13 (dd, J=6.9, 1.7 Hz, 3H), 1.06 (dd, J=6.9, 1.8 Hz, 3H), 1.00 (dd, J=6.8, 2.0 Hz, 3H), 0.85 (td, J=7.4, 3.3 Hz, 9H) ppm. ¹³C NMR (151 MHz, Chloroform-d) δ 148.85, 147.30, 145.94, 142.38, 141.57, 137.41, 137.06, 135.33, 133.68, 133.60, 133.55, 133.47, 132.00, 130.93, 128.57, 128.33 (dd, J_{CP}=6.4, 3.1 Hz), 128.23, 128.16, 32.47, 30.92, 29.47, 25.56, 24.31 (d, J=11.3 Hz), 23.71, 22.20, 21.97 ppm. ³¹P NMR (243 MHz, CDCl₃) δ=−17.94 ppm.

### Synthesis of Ligand TIP-Mes*Phine

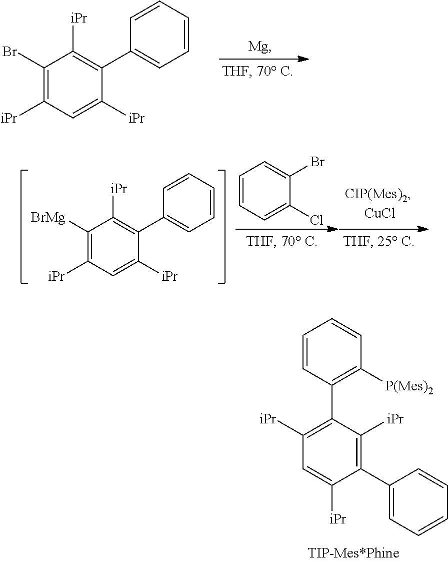

### Dimesityl(2′,4′,6′-triisopropyl-[1,1′:3′,1″-terphenyl]-2-yl)phos phane (TIP-Mes*Phine)

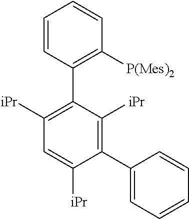

To an oven-dried flask fitted with a septum was sequentially added activated Mg with DIBAL (52.8 mg, 2.2 mmol, 2.2 equiv), 1 mL of THF and 3-bromo-2,4,6-triisopropyl-1,1′-biphenyl (360 mg, 1 mmol, 1.0 equiv). The mixture was then heated to 70° C. in an oil bath and stirred for 2 h prior to the dropwise addition of 2-bromochlorobenzene (237 mg, 1.2 mmol, 1.2 equiv) at 70° C., after which was stirred for another 3 h. At this time, the mixture was cooled to room temperature and was transferred to another oven-dried flask with charge of CuCl (9.9 mg, 0.1 mmol, 10 mol %) and LiBr (17.4 mg, 0.2 mmol, 20 mol %). Additional of 1 mL anhydrous THF was rinsed and also was transferred. Chlorodimesitylphosphane (P(Mes)₂Cl)(335 mg, 1.1 mmol, 1.1 equiv) was subsequently added at 70° C. and stirred 24 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (188 mg, 30%), labelled as TIP-Mes*Phine. ¹H NMR (600 MHz, Chloroform-d) δ 7.53-7.46 (m, 1H), 7.36-7.22 (m, 6H), 7.22-7.14 (m, 2H), 7.06 (s, 1H), 6.74 (s, 2H), 2.52 (td, J=7.1, 1.6 Hz, 1H), 2.45 (td, J=6.9, 1.7 Hz, 1H), 2.25 (d, J=5.2 Hz, 7H), 1.98 (s, 5H), 1.92 (s, 5H), 1.09 (ddd, J=14.2, 6.9, 1.6 Hz, 7H), 0.95 (dd, J=6.7, 1.6 Hz, 3H), 0.86 (ddd, J=11.5, 6.9, 1.6 Hz, 7H). ppm. ¹³C NMR (151 MHz, CDCl₃) 147.15, 146.65, 146.42, 144.51, 144.43, 144.41, 144.33, 143.00, 141.76, 138.09, 137.98, 136.89, 136.65, 136.36, 136.26, 134.23, 131.96, 131.26, 131.21, 130.53, 129.92, 129.90, 129.88, 129.85, 129.68, 129.53, 126.93, 126.61, 126.52, 126.20, 125.22, 119.08, 32.16, 30.83, 29.37, 26.23, 24.69, 24.66, 23.89, 23.73, 23.64, 23.55, 22.56, 22.23, 22.21, 20.79, 20.77 ppm. ³¹P NMR (243 MHz, CDCl₃) δ=−24.41 ppm.

### Synthesis of Ligand TIP-tBu*Phine-CF₃

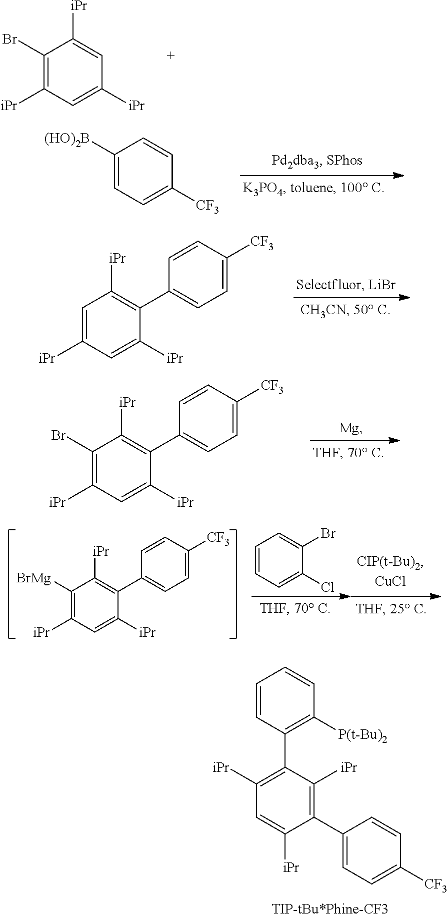

### 2,4,6-triisopropyl-4′-(trifluoromethyl)-1,1′-biphenyl

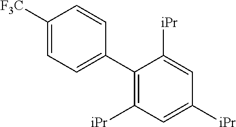 [SMILES: CC(C)c1cc(C(C)C)c(-c2ccc(C(F)(F)F)cc2)c(C(C)C)c1]

To a 50 mL oven-dried flask was added 2-bromo-1,3,5-triisopropylbenzene (10.14 g, 0.036 mol, 1.0 equiv.), (4-(trifluoromethyl)phenyl)boronic acid (7.32 g, 0.06 mol, 1.6 equiv.), K₃PO₄ (17.0 g, 0.80 mol, 2.2 equiv), Pd₂(dba)₃ (0.366 g, 0.4 mmol) and SPhos (0.328 g, 0.8 mmol). The flask was evacuated and back-filled with argon three times prior to 40 mL of dry THF and 40 mL of dry toluene was added via syringe. The mixture was then stirred and heated in a 100° C. oil bath for 16 hr. Upon reaction completion, as determined by GC-MS, the mixture was filtered through silica and washed with EtOAc. The filtrate was collected and the solvent was removed under vacuum. The crude residue was purified by flash chromatography on silica gel using an EtOAc/hexanes mixture (0 to 10% EtOAc) as the eluent to get a white solid (11.1 g, 89%). ¹H NMR (600 MHz, Chloroform-d) δ 7.42 (dd, J=8.1, 6.8 Hz, 2H), 7.39-7.34 (m, 1H), 7.23-7.19 (m, 2H), 7.09 (s, 1H), 2.97 (p, J=7.0 Hz, 1H), 2.62 (ddd, J=12.7, 7.7, 6.4 Hz, 2H), 1.34 (dd, J=7.0, 0.9 Hz, 5H), 1.11 (dd, J=6.9, 0.9 Hz, 11H) ppm. ¹³C NMR (151 MHz, CDCl₃) δ 147.82, 146.51, 140.84, 137.08, 129.80, 127.91, 126.40, 120.53, 77.26, 77.05, 76.84, 34.28, 30.28, 24.25, 24.13 ppm.

### 3-bromo-2,4,6-triisopropyl-4′-(trifluoromethyl)-1,1′-biphenyl

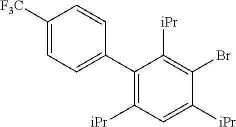 [SMILES: CC(C)c1cc(C(C)C)c(-c2ccc(C(F)(F)F)cc2)c(C(C)C)c1Br]

To a 250 mL oven-dried flask was added 2,4,6-triisopropyl-4′-(trifluoromethyl)-1,1′-biphenyl (10.0 g, 0.036 mol, 1.0 equiv.), Selectfluor (14.5 g, 0.041 mol), LiBr (3.6 g, 0.041 mol). The flask was evacuated and back-filled with argon three times prior to 180 mL of anhydrous acetonitrile was added via syringe. The mixture was then stirred at room temperature overnight. Upon reaction completion, the solvent was removed and DCM was added to the crude. The mixture was filtered; the filtrate was wash with sodium thiosulfate. The organic layer was collected and dry with MgSO₄. The crude was then purified with column with Hexane as eluents as the eluent to get a white solid (13.8 g, 90%). ¹H NMR (600 MHz, Chloroform-d) δ 7.44-7.35 (m, 3H), 7.16 (s, 2H), 3.62 (p, J=6.8 Hz, 1H), 3.08-2.88 (m, 1H), 2.49 (p, J=6.9 Hz, 1H), 1.32 (d, J=6.9 Hz, 12H), 1.07 (d, J=6.9 Hz, 6H) ppm.

### Di-tert-butyl(2′,4′,6′-triisopropyl-4″-(trifluoromethyl)-[1,1′:3′,1″-terphenyl]-2-yl)phosphane (TIP-tBu*Phine-CF₃)

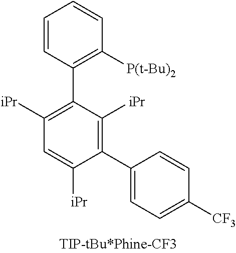

To an oven-dried flask fitted with a septum was sequentially added activated Mg with DIBAL (105.4 mg, 4.4 mmol, 2.2 equiv), 1 mL of THF and 3-bromo-2,4,6-triisopropyl-4′-(trifluoromethyl)-1,1′-biphenyl (855 mg, 2 mmol, 1.0 equiv). The mixture was then heated to 70° C. in an oil bath and stirred for 2 h prior to the dropwise addition of 2-bromochlorobenzene (421 mg, 2.2 mmol, 1.1 equiv) at 70° C., after which was stirred for another 3 h. At this time, the mixture was cooled to room temperature and was transferred to another oven-dried flask with charge of CuCl (214 mg, 2 mmol, 1.0 equiv). Additional of 1 mL anhydrous THF was rinsed and also was transferred. P(t-Bu)₂Cl (398 mg, 2.2 mmol, 1.1 equiv) was subsequently added at 70° C. and stirred 24 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (318 mg, 28%), labelled as TIP-tBu*Phine-CF₃. ¹H NMR (600 MHz, Chloroform-d) δ 7.87 (d, J=7.3 Hz, 1H), 7.66-7.62 (m, 1H), 7.60 (d, J=8.1 Hz, 1H), 7.42 (dd, J=13.4, 7.7 Hz, 2H), 7.34 (s, 2H), 7.17 (s, 1H), 2.81 (t, J=7.4 Hz, 1H), 2.45 (dd, J=9.9, 4.2 Hz, 1H), 2.35 (p, J=6.8 Hz, 1H), 1.24 (d, J=6.8 Hz, 3H), 1.17 (t, J=12.7 Hz, 17H), 1.12 (d, J=6.8 Hz, 3H), 1.06 (d, J=6.9 Hz, 3H), 1.01 (d, J=6.7 Hz, 3H), 0.91 (d, J=7.1 Hz, 3H) ppm. ¹³C NMR (151 MHz, Chloroform-d) δ 149.31 (d, J_{CP}=33.1 Hz), 146.69, 146.55, 146.20, 142.14, 138.22 (d, J_{CP}=4.5 Hz), 136.89 (d, J_{CP}=30.9 Hz), 136.17, 135.61, 132.53, 132.31 (d, J_{CP}=7.0 Hz), 130.98, 129.03, 128.22, 127.54, 125.37, 123.98 (d, J_{CP}=3.9 Hz), 123.76 (d, J_{CP}=3.7 Hz), 119.35, 32.75 (dd, J_{CP}=25.3, 13.8 Hz), 32.29 (d, J_{CP}=2.5 Hz), 31.00 (t, J_{CP}=15.1 Hz), 29.43, 26.45, 24.31, 24.22, 23.49, 22.79 ppm. ³¹P NMR (243 MHz, CDCl₃) δ=22.42 ppm. ¹⁹F NMR (376 MHz; CDCl₃) δ=−61.91 ppm.

### Synthesis of Ligand TIP-Cy*Phine-CF₃

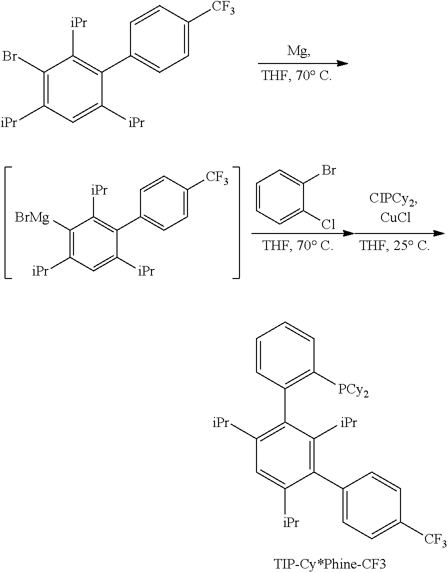

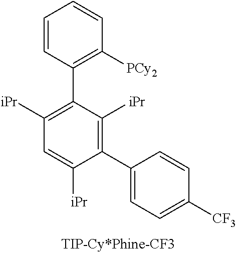

Dicyclohexyl(2′,4′,6′-triisopropyl-4″-(trifluoromethyl)-[1,1′:3′,1″-terphenyl]-2-yl)phosphane (TIP-Cy*Phine-CF₃). To an oven-dried flask fitted with a septum was sequentially added activated Mg with DIBAL (54.8 mg, 2.2 mmol, 2.2 equiv), 1 mL of THF and 3-bromo-2,4,6-triisopropyl-4′-(trifluoromethyl)-1,1′-biphenyl (428 mg, 1 mmol, 1.0 equiv). The mixture was then heated to 70° C. in an oil bath and stirred for 2 h prior to the dropwise addition of 2-bromochlorobenzene (211 mg, 1.1 mmol, 1.1 equiv) at 70° C., after which was stirred for another 3 h. At this time, the mixture was cooled to room temperature and was transferred to another oven-dried flask with charge of CuCl (5 mg, 0.05 mmol, 5 mol %). Additional of 1 mL anhydrous THF was rinsed and also was transferred. PCy₂Cl (245 mg, 1.1 mmol, 1.1 equiv) was subsequently added at room temperature and stirred 24 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (360 mg, 58%), labelled as TIP-Cy*Phine-CF₃. ¹H NMR (400 MHz, Benzene-d₆) δ 7.71 (d, J=6.7 Hz, 1H), 7.63 (s, 1H), 7.58-7.47 (m, 4H), 7.46-7.39 (m, 2H), 3.21 (p, J=7.1 Hz, 1H), 2.86 (p, J=6.9 Hz, 1H), 2.67 (p, J=6.8 Hz, 1H), 2.15 (d, J=12.5 Hz, 3H), 2.06-1.77 (m, 16H), 1.65 (d, J=6.9 Hz, 3H), 1.51-1.38 (m, 3H), 1.33 (dd, J=8.6, 6.7 Hz, 8H), 1.24 (d, J=7.2 Hz, 3H), 0.95 (d, J=7.2 Hz, 3H) ppm. ¹³C NMR (151 MHz, Benzene-d₆) δ 148.59 (d, J_{CP}=31.3 Hz), 146.17, 139.88 (d, J_{CP}=6.0 Hz), 138.27, 136.24 (d, J_{CP}=20.9 Hz), 135.44, 134.14, 132.72 (d, J_{CP}=2.8 Hz), 130.67 (d, J_{CP}=5.9 Hz), 130.07, 129.82, 129.03, 128.66, 126.51, 125.54 (dd, J_{CP}=12.6, 3.8 Hz), 34.33 (d, J_{CP}=16.5 Hz), 34.01 (d, J_{CP}=16.1 Hz), 31.17-30.69 (m), 30.62, 29.99 (d, J_{CP}=14.9 Hz), 29.75 (d, J_{CP}=13.4 Hz), 28.12-26.87 (m), 26.59 (d, J_{CP}=9.2 Hz), 21.57, 20.62, 20.06 ppm. ³¹P NMR (243 MHz, CDCl₃) δ=−11.67 ppm. ¹⁹F NMR (376 MHz, CDCl₃) δ=−61.92 ppm.

### Synthesis of Ligand TIP-tBu*Phine-OMe

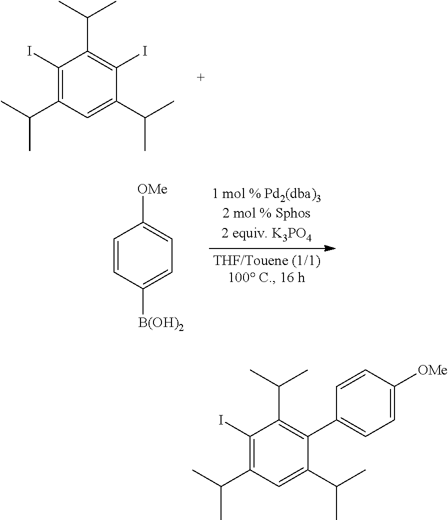

### 3-iodo-2,4,6-triisopropyl-4′-methoxy-1,1′-biphenyl

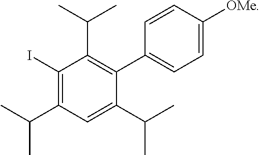 [SMILES: COc1ccc(-c2c(C(C)C)cc(C(C)C)c(I)c2C(C)C)cc1]

To an oven-dried 50 mL flask, 2,4-diiodo-1,3,5-triisopropylbenzene (2.5 g, 5.5 mmol, 1.0 equiv.), 4-methoxyphenylboronic acid (1.25 g, 8.3 mmol, 1.5 equiv.), K₃PO₄ (2.33 g, 11 mmol, 2 equiv.), Pd₂(dba)₃ (51 mg, 0.005 mmol, 1 mol %) and Sphos (45 mg, 0.011 mmol, 2 mol %) were added. The flask was then vacated for 5 min and back-filled with argon prior to the addition of anhydrous THF (5.5 mL) and toluene (5.5 mL). The resulting mixture was heated to 100° C. in a preheated oil bath with vigorous stirring for 16 h. At this time, the mixture was cooled to room temperature and was filtered through a pad of silica and washed with ethyl acetate. The filtrate was collected and the solvent was removed via rotavap. The crude was purified through silica column with gradient solvent of pure hexane to ethyl acetate/hexane (1:10) to get a white solid (720 mg, 30%). ¹H NMR (600 MHz, CDCl₃) δ=7.09-7.03 (m, 3H), 6.95-6.90 (m, 2H), 3.88 (s, 3H), 3.52 (q, J=6.6 Hz, 1H), 3.16 (t, J=6.52 Hz, 1H), 2.51-0.49 (m, 1H), 1.37 (d, J=7.2 Hz, 2H), 1.31 (d, J=6.6 Hz, 6H), 1.04 (t, J=7.2 Hz, 6H), 0.89 (d, J=7.2 Hz, 3H) ppm. ¹³C NMR (150 MHz, CDCl₃) δ=158.49, 150.74, 150.46, 148.71, 146.71, 138.33, 132.22, 132.01, 129.84, 120.67, 120.52, 113.61, 112.50, 109.99, 55.19, 42.56, 40.39, 38.97, 34.09, 30.47, 29.08, 24.05, 23.95, 23.56, 23.51, 22.79, 20.09 ppm.

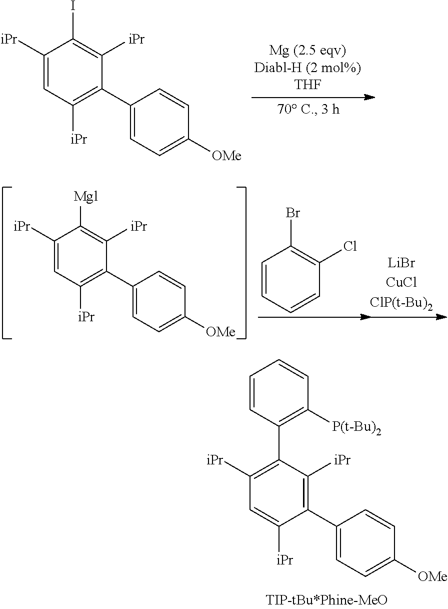

### di-tert-butyl(2′,4′,6′-triisopropyl-4″-methoxy-[1,1′:3′,1″-terphenyl]-2-yl)phosphane (TIP-tBu*Phine-OMe)

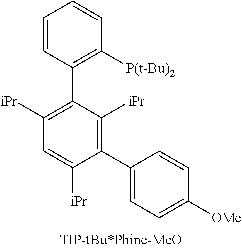

To an oven-dried flask fitted with a septum was sequentially added activated Mg with DIBAL (54.8 mg, 2.2 mmol, 2.2 equiv), 1 mL of THF and 3-iodo-2,4,6-triisopropyl-4′-methoxy-1,1′-biphenyl (435 mg, 1 mmol, 1.0 equiv). The mixture was then heated to 70° C. in an oil bath and stirred for 3 h prior to the dropwise addition of 2-bromochlorobenzene (286 mg, 1.5 mmol, 1.5 equiv) at 70° C., after which was stirred for another 16 h. At this time, the mixture was cooled to room temperature. On a separated oven-dried 20 mL flask was charged with CuCl (10 mg, 0.1 mmol, 10 mol %) and LiBr (17.4 mg, 0.2 mmol, 20 mol %) and anhydrous toluene (2 mL) and was allowed to stir for 5 min. The Grignard reagent was diluted with 5 mL of anhydrous toluene and was transferred into the flask followed by addition of P(t-Bu)₂Cl (200 mg, 1.2 mmol, 1.2 equiv.). The flask was capped under argon and the resulting mixture was heated to 120° C. in a preheated oil bath with vigorous stirring for 18 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in hexane/MeOH affords pure white solid (165 mg, 31%), labelled as TIP-tBu*Phine-OMe. ¹H NMR (400 MHz, CDCl₃) δ 7.85 (d, J=7.62 Hz, 1H), 7.44 (s, 1H), 7.43-7.27 (m, 1H), 7.27-7.26 (m, 1H), 7.25-7.22 (m, 1H), 7.22-7.17 (m, 1H), 7.12-7.11 (m, 1H), 6.78-6.75 (m, 2H), 3.30 (s, 3H), 3.20 (q, J=7.08 Hz 1H), 2.80 (q, J=6.84, 1H), 2.66 (q, J=6.72, 1H), 1.39 (d, J=6.78 Hz, 3H), 1.20-1.13 (m, 30H), 0.92 (d, J=7.2 Hz, 3H) ppm. ¹³C NMR (151 MHz, CDCl₃) δ 148.59 (d, J_{CP}=31.3 Hz), 158.58, 150.11, 149.89, 147.91, 146.33, 143.03, 138.49, 137.41, 137.19, 136.98, 136.14, 133.07, 132.58, 132.54, 131.62, 125.39, 119.29, 112.29, 112.39, 54.28, 32.82, 32.80, 32.70, 32.61, 32.53, 32.43, 31.20, 31.06, 30.96, 30.69, 30.59, 29.52, 26.69, 24.56, 24.48, 24.11, 23.60, 22.8 ppm. ³¹P NMR (243 MHz, CDCl₃) δ=21.57 ppm.

### TOM/TOP Series

### Synthesis of Ligand TOM-Cy*Phine

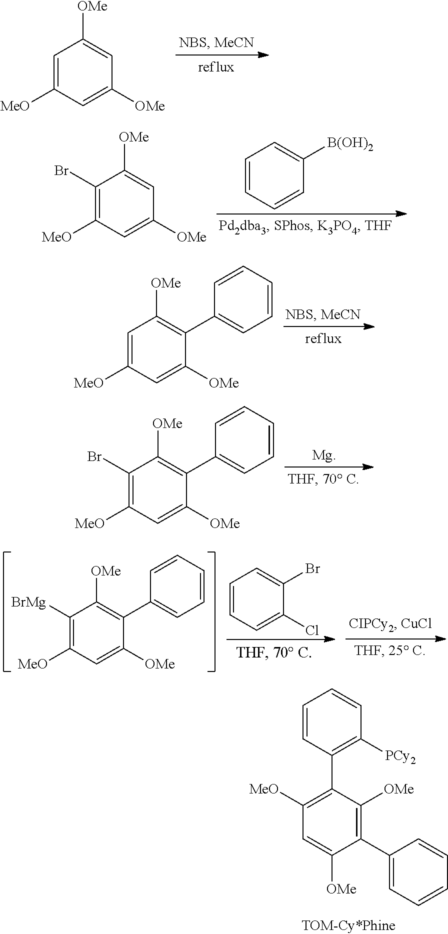

### 2-bromo-1,3,5-trimethoxybenzene

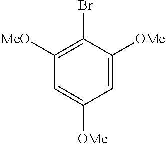 [SMILES: COc1cc(OC)c(Br)c(OC)c1]

To an oven-dried flask fitted with a septum was sequentially added 1,3,5-trimethoxybenzene (1.68 g, 10 mmol, 1.0 equiv.), N-Bromosuccinimide (1.78 g, 10 mmol, 1.0 equiv.) and 10 mL anhydrous acetonitrile. The resulting mixture was heated to 800° C. for 5 h under vigorous stirring. At this time, the mixture was cooled to room temperature and filtered. The solvent was removed under vacuum to get a crude product which was purified through silica column with 8 v % of ethyl acetate in hexane to get a white solid (1.48 g, 60%). ¹H NMR (600 MHz, Chloroform-d) δ 6.18 (s, 2H), 3.89 (s, 6H), 3.83 (s, 3H) ppm. 2,4,6-trimethoxy-1,1′-biphenyl.

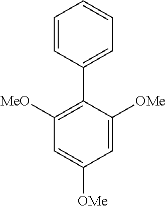 [SMILES: COc1cc(OC)c(-c2ccccc2)c(OC)c1]

To a 50 mL oven-dried flask was added 2-bromo-1,3,5-trimethoxybenzene (4.9 g, 20 mmol, 1.0 equiv.), phenylboronic acid (3.6 g, 30 mmol, 1.5 equiv.), K₃PO₄ (8.5 g, 40 mmol, 2.0 equiv), Pd₂(dba)₃ (0.368 g, 0.4 mmol) and SPhos (0.328 g, 0.8 mmol). The flask was evacuated and back-filled with argon three times prior to 12 mL of dry THF and 12 mL of dry toluene was added via syringe. The mixture was then stirred and heated in a 100° C. oil bath for 16 hr. Upon reaction completion, as determined by GC-MS, the mixture was filtered through silica and washed with EtOAc. The filtrate was collected and the solvent was removed under vacuum. The crude residue was purified by flash chromatography on silica gel using an EtOAc/hexanes mixture (0 to 10% EtOAc) as the eluent to get a white solid 2,4,6-trimethoxy-1,1′-biphenyl (3.5 g, 72%). ¹H NMR (600 MHz, Chloroform-d) δ 7.50-7.28 (m, 5H), 6.26 (s, 2H), 3.89 (s, 3H), 3.74 (s, 6H) ppm.

### 3-bromo-2,4,6-trimethoxy-1,1′-biphenyl

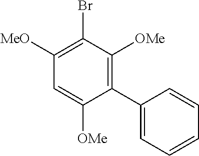 [SMILES: COc1cc(OC)c(-c2ccccc2)c(OC)c1Br]

To an oven-dried flask fitted with a septum was sequentially added 2,4,6-trimethoxy-1,1′-biphenyl (2.53 g, 10.4 mmol, 1.0 equiv.), N-Bromosuccinimide (1.84 g, 10.4 mmol, 1.0 equiv.) and 20 mL anhydrous acetonitrile. The resulting mixture was stirred at room temperature overnight At this time, the mixture was cooled to room temperature and filtered. The solvent was removed under vacuum to get a crude product which was purified through silica column with 8 v % of ethyl acetate in hexane to get a white solid (3.06 g, 95%). ¹H NMR (600 MHz, Chloroform-d) δ 7.49-7.20 (m, 5H), 6.40 (s, 1H), 3.95 (s, 3H), 3.74 (s, 3H), 3.37 (s, 3H) ppm.

### Dicyclohexyl(2′,4′,6′-trimethoxy-[1,1′:3′,1″-terphenyl]-2-yl) phosphane (TOM-Cy*Phine)

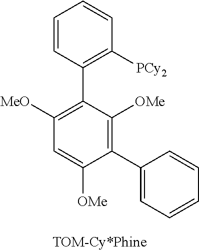

To an oven-dried flask fitted with a septum was sequentially added activated Mg with DIBAL (52.8 mg, 2.2 mmol, 2.2 equiv), 1 mL of THF and 3-bromo-2,4,6-trimethoxy-1,1′-biphenyl (323 mg, 1 mmol, 1.0 equiv). The mixture was then heated to 70° C. in an oil bath and stirred for 2 h prior to the dropwise addition of 2-bromochlorobenzene (237 mg, 1.2 mmol, 1.2 equiv) at 70° C., after which was stirred for another 3 h. At this time, the mixture was cooled to room temperature and was transferred to another oven-dried flask with charge of CuCl (9.9 mg, 0.1 mmol, 10 mol %). Additional of 1 mL anhydrous THF was rinsed and also was transferred. PCy₂Cl (245 mg, 1.1 mmol, 1.1 equiv) was subsequently added and stirred 16 h at room temperature. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (232 mg, 45%), labelled as TOM-Cy*Phine. ¹H NMR (600 MHz, Chloroform-d) δ 7.61 (d, J=7.6 Hz, 1H), 7.48-7.40 (dd, J=8.4, 6.9 Hz, 3H), 7.35 (td, J=7.4, 1.6 Hz, 3H), 7.32-7.23 (m, 2H), 6.40 (s, 1H), 3.82 (s, 3H), 3.78 (s, 3H), 3.01 (s, 3H), 1.95-1.58 (m, 11H), 1.39-0.94 (m, 11H) ppm. ¹³C NMR (151 MHz, Chloroform-d) δ 157.46, 156.91 (d, J_{CP}=9.9 Hz), 143.02, 142.80, 136.87, 136.75, 134.71, 132.37 (d, J_{CP}=3.7 Hz), 131.19, 127.95, 127.61, 126.35, 126.16, 118.18, 116.25, 90.45, 60.16, 55.62, 55.24, 34.27 (t, J_{CP}=13.7 Hz), 30.29 (d, J_{CP}=17.1 Hz), 30.02 (d, J_{CP}=12.7 Hz), 29.79 (d, J_{CP}=14.9 Hz), 29.30 (d, J_{CP}=8.8 Hz), 27.59-27.35 (m), 26.56 (d, J_{CP}=15.9 Hz) ppm. ³¹P NMR (243 MHz, CDCl₃) δ=−8.55 ppm.

### Synthesis of Ligand TOM-tBu*Phine

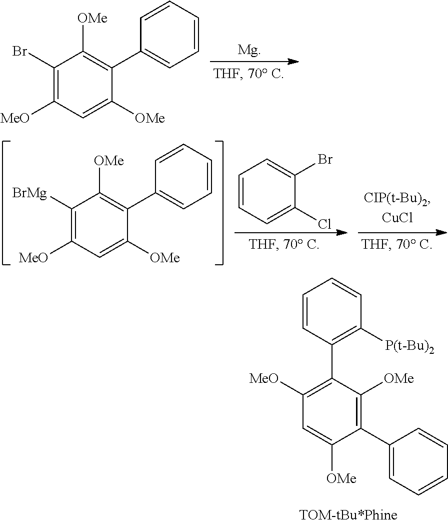

### Di-tert-butyl(2′,4′,6′-trimethoxy-[1,1′:3′,1″-terphenyl]-2-yl)phosphane (TOM-tBu*Phine)

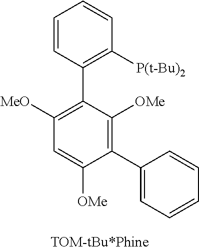

To an oven-dried flask fitted with a septum was sequentially added activated Mg with DIBAL (52.8 mg, 2.2 mmol, 2.2 equiv), 1 mL of THF and 3-bromo-2,4,6-trimethoxy-1,1′-biphenyl (323 mg, 1 mmol, 1.0 equiv). The mixture was then heated to 70° C. in an oil bath and stirred for 2 h prior to the dropwise addition of 2-bromochlorobenzene (237 mg, 1.2 mmol, 1.2 equiv) at 70° C., after which was stirred for another 3 h. At this time, the mixture was cooled to room temperature and was transferred to another oven-dried flask with charge of CuCl (99 mg, 1 mmol, 1.0 equiv). Additional of 1 mL anhydrous THF was rinsed and also was transferred. P(t-Bu)₂Cl (199 mg, 1.1 mmol, 1.1 equiv) was subsequently added and stirred 16 h at 70° C. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (121 mg, 26%). ¹H NMR (600 MHz, Chloroform-d) δ 7.87 (d, J=7.7 Hz, 1H), 7.48-7.43 (m, 2H), 7.42-7.36 (m, 3H), 7.34-7.27 (m, 2H), 7.25 (ddd, J=7.8, 3.8, 1.6 Hz, 1H), 6.39 (s, 1H), 3.84 (s, 3H), 3.76 (s, 3H), 2.98 (s, 3H), 1.24 (d, J=11.6 Hz, 9H), 1.18 (d, J=11.4 Hz, 9H) ppm. ¹³C NMR (151 MHz, Chloroform-d) δ 157.28, 156.89, 156.68, 143.81, 143.58, 138.38 (d, J_{CP}=25.9 Hz), 135.30 (d, J_{CP}=3.7 Hz), 134.72, 131.37 (d, J_{CP}=6.6 Hz), 131.20, 127.78, 127.59, 126.28, 125.43, 118.92, 116.16, 90.54, 59.84, 55.65, 55.03, 32.03 (dd, J_{CP}=24.7, 15.3 Hz), 30.68 (dd, J_{CP}=15.6, 10.3 Hz) ppm. ³¹P NMR (243 MHz, CDCl₃) δ=24.17 ppm.

### Synthesis of Ligand 25OMe-TOM-Cy*Phine

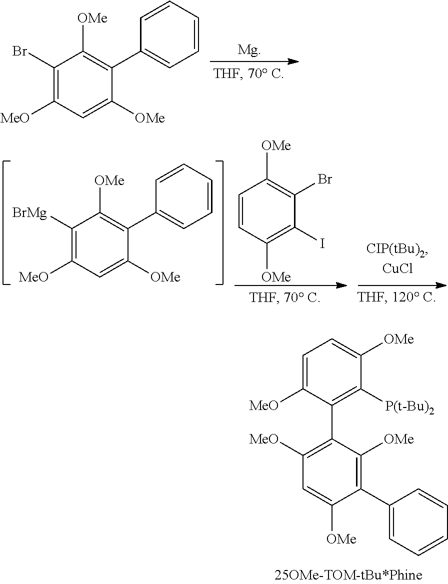

### Di-tert-butyl(2′,3,4′,6,6′-pentamethoxy-[1,1′:3′,1″-terphenyl]-2-yl)phosphane (25OMe-TOM-tBu*Phine)

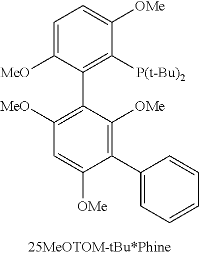

To an oven-dried flask fitted with a septum was sequentially added activated Mg with DIBAL (52.8 mg, 2.2 mmol, 2.2 equiv), 1 mL of THF and 3-bromo-2,4,6-trimethoxy-1,1′-biphenyl (323 mg, 1 mmol, 1.0 equiv). The mixture was then heated to 70° C. in an oil bath and stirred for 2 h prior to the dropwise addition of 2-bromo-3-iodo-1,4-dimethoxybenzene (410 mg, 1.2 mmol, 1.2 equiv) dissolved in anhydrous THF at 70° C., after which was stirred for another 12 h. At this time, the mixture was cooled to room temperature and was transferred to another oven-dried flask with charge of CuCl (99 mg, 1 mmol, 1.0 equiv). Additional of 1 mL anhydrous THF was rinsed and also was transferred. P(t-Bu)₂Cl (199 mg, 1.1 mmol, 1.1 equiv) was subsequently added at 120° C. and stirred 24 h. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (157 mg, 30%), labelled as 25OMe-TOM-tBu*Phine. ¹H NMR (600 MHz, Chloroform-d) δ 7.48 (dd, J=8.2, 1.4 Hz, 1H), 7.39 (t, J=7.7 Hz, 2H), 7.29 (m, 2H), 6.95 (d, J=9.0 Hz, 1H), 6.82 (d, J=9.0 Hz, 1H), 6.46 (s, 1H), 3.88 (s, 3H), 3.82 (s, 3H), 3.81 (s, 3H), 3.74 (s, 3H), 3.07 (s, 3H), 1.42 (d, J=14.6 Hz, 9H), 1.28 (d, J=15.0 Hz, 9H) ppm. ¹³C NMR (151 MHz, CDCl₃) δ 158.02, 157.04, 156.76, 152.75, 152.41, 134.33, 131.74, 131.12, 127.66, 126.49, 117.30, 116.96, 111.53, 109.98, 96.49, 91.80, 77.27, 77.06, 76.85, 60.13, 56.84, 56.76, 56.12, 55.76, 38.57, 38.46, 31.13, 31.10, 28.40, 28.33, 28.19, 28.13, 25.75 ppm. ³¹P NMR (243 MHz, CDCl₃) δ=65.94 ppm.

### Synthesis of Ligand TOP-Cy*Phine

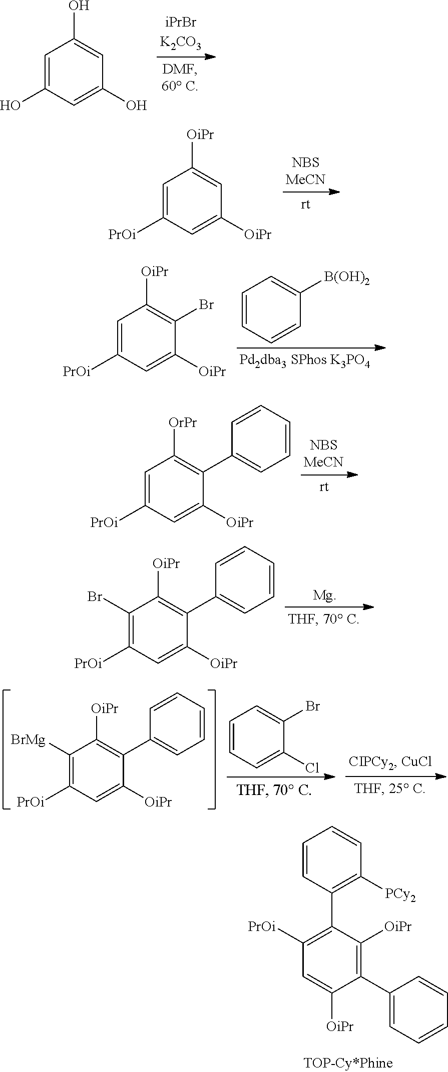

### 1,3,5-triisopropoxybenzene

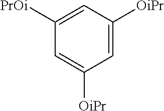

Phloroglucinol (1 equiv), K₂CO₃ (6 equiv), DMF (2 ml/mmol) and 2-bromo-propane (6 equiv) were added to a reaction vial and a screw cap was fitted to it. The reaction mixture was stirred under air in a closed system at 100° C. for 24 h. The resulting mixture was cooled to room temperature and H₂O (20 mL) was added and the product was extracted with Et₂O (3×20 mL).

The organic layer was washed with H₂O (4×20 mL), dried (Mg₂SO₄) and the solvent was removed under reduced pressure. The crude product was purified by silica-gel column chromatography to afford the pure product (92:8 petroleum ether/ethyl acetate) in 50% yield. ¹H NMR (600 MHz, CDCl₃): δ 6.03 (s, 3H), 1.32 (d, J=6.0 Hz, 18H). ¹³C NMR (600 MHz, CDCl₃): δ 159.7, 96.1, 69.8, 22.1.

### 2-bromo-1,3,5-triisopropoxybenzene

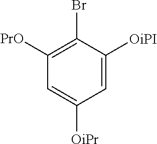

To an oven-dried flask fitted with a septum was sequentially added 1,3,5-triisopropylbenzene (0.25 g, 1 mmol, 1.0 equiv.), N-Bromosuccinimide (0.178 g, 0.1 mmol, 1.0 equiv.) and 2 mL anhydrous acetonitrile. The resulting mixture was stirred at room temperature overnight. The solvent was removed under vacuum to get a crude product which was purified through silica column with gradient solvent from pure hexane to 4 v % of ethyl acetate in hexane to get a pure product (0.33 g, 90%). ¹H NMR (600 MHz, Chloroform-d) δ 6.15 (s, 2H), 1.36 (d, J=6.1 Hz, 12H), 1.32 (d, J=6.0 Hz, 6H) ppm. 2,4,6-tripropoxy-1,1′-biphenyl.

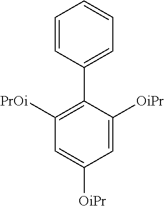

To a 50 mL oven-dried flask was added 2-bromo-1,3,5-triisopropoxybenzene (1 g, 3 mmol, 1.0 equiv.), phenylboronic acid (0.55 g, 4.5 mmol, 1.5 equiv.), K₃PO₄ (1.27 g, 6 mmol, 2.0 equiv), Pd₂(dba)₃ (0.0.27 g, 0.03 mmol) and SPhos (0.025 g, 0.06 mmol). The flask was evacuated and back-filled with argon three times prior to 12 mL of dry THF and 12 mL of dry toluene was added via syringe. The mixture was then stirred and heated in a 100° C. oil bath for 16 hr. Upon reaction completion, as determined by GC-MS, the mixture was filtered through silica and washed with EtOAc. The filtrate was collected and the solvent was removed under vacuum. The crude residue was purified by flash chromatography on silica gel using an petroleum ether/ethyl acetate (92:8) as the eluent to get a white solid (0.81 g, 82%). ¹H NMR (600 MHz, Chloroform-d) δ 7.45-7.42 (m, 1H), 7.36-7.29 (m, 4H), 6.21 (s, 2H), 1.37 (d, J=6.1 Hz, 6H), 1.14 (d, J=6.1 Hz, 12H) ppm.

### 3-bromo-2,4,6-triisopropoxy-1,1′-biphenyl

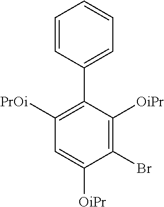

To an oven-dried flask fitted with a septum was sequentially added 2,4,6-tripropoxy-1,1′-biphenyl (0.8 g, 2.45 mmol, 1.0 equiv.), N-Bromosuccinimide (0.44 g, 2.45 mmol, 1.0 equiv.) and 5 mL anhydrous acetonitrile. The resulting mixture was stirred at room temperature overnight. At this time, the mixture was cooled to room temperature and filtered. The solvent was removed under vacuum to get a crude product which was purified through silica column with 6 v % of ethyl acetate in hexane to get a white solid (0.9 g, 72%). ¹H NMR (600 MHz, Chloroform-d) δ 7.41-7.31 (m, 5H), 6.41 (s, 2H), 1.41 (d, J=6.1 Hz, 6H), 1.13 (d, J=6.1 Hz, 6H), 0.94 (d, J=6.2 Hz, 6H) ppm.

### Diphenyl(2′,4′,6′-trimethoxy-[1,1′:3′,1″-terphenyl]-2-yl)phos-phane (TOP-Cy*Phine)

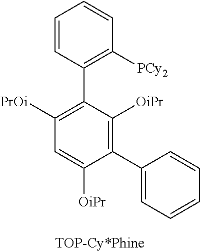

To an oven-dried flask fitted with a septum was sequentially added activated Mg with DIBAL (52.8 mg, 2.2 mmol, 2.2 equiv), 1 mL of THF and 3-bromo-2,4,6-triisopropyl-1,1′-biphenyl (348 mg, 1 mmol, 1.0 equiv). The mixture was then heated to 70° C. in an oil bath and stirred for 2 h prior to the dropwise addition of 2-bromochlorobenzene (237 mg, 1.2 mmol, 1.2 equiv) at 70° C., after which was stirred for another 3 h. At this time, the mixture was cooled to room temperature and was transferred to another oven-dried flask with charge of CuCl (9.9 mg, 0.1 mmol, 10 mol %). Additional of 1 mL anhydrous THF was rinsed and also was transferred. PCy₂Cl (245 mg, 1.1 mmol, 1.1 equiv) was subsequently added and stirred 24 h at room temperature. After the reaction was complete, as determined by ³¹P NMR and GC analysis, ethyl acetate was added and the mixture was washed several times with 50 mL of 30% aq NH₄OH. The organic layer was separated, dried with MgSO₄, filtered and concentrated to give the crude product; recrystallization in MeOH affords pure white solid (175 mg, 36%), labelled as TOP-Cy*Phine. ³¹P NMR (243 MHz, CDCl₃) δ=−8.15 ppm.

### Modified TIP Series

### Synthesis of Ligand 2MeOTIP-tBu*Phine

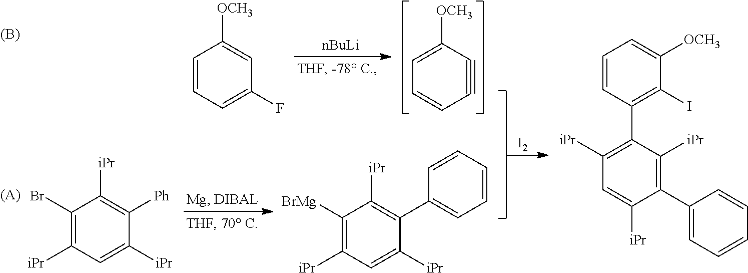

### 2-iodo-2′,4′,6′-triisopropyl-3-methoxy-1,1′:3′,1″-terphenyl

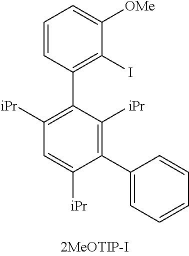 [SMILES: COc1cccc(-c2c(C(C)C)cc(C(C)C)c(-c3ccccc3)c2C(C)C)c1I]

A 10 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 2-bromo-1,3,5-triisopropylbenzene (566 mg, 2 mmol) was added potion-by-potion with vigorous stirring. The tube was capped with a rubber septum and was heated to 70° C. for 2 h (A). In another 10 mL oven-dried Schlenk tube was charged with 1-fluoro-3-methoxybenzene (126.2 mg, 1 mmol) and anhydrous THF (3.5 mL). The tube was cooled to −78° C. and 1.6 M n-BuLi solution in hexane (0.64 mL, 1 mmol) was added dropwise over 10 min under vigorous stirring. The mixture was stirred at −78° C. for 40 min (B). A was transferred into to B by syringe over 15 min at −78° C. An additional 1 mL of THF was used to rinse the reaction and was also transferred to B. The combined reaction mixture was stirred at −78° C. for another 1 h and then slowly warmed to room temperature and vigorously stirred for overnight. At this time, the mixture was cooled to 0° C. using ice bath, and iodine (762 mg, 3 mmol) in 4 mL anhydrous THF was added drop-wise over 5 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (10 mL×3) and the combined organic phases were dried over anhydrous MgSO₄. After concentration, the crude product was purified by silica gel chromatography (EtOAc/petroleum ether=1/20) followed by crystallization in methanol to get a white solid (205 mg, 40%), labeled as 2MeOTIP-I. ¹H NMR (600 MHz, Chloroform-d) δ 7.45-7.37 (m, 4H), 7.36-7.32 (m, 3H), 7.00-6.96 (m, 1H), 6.85 (dd, J=8.2, 1.4 Hz, 1H), 4.02 (s, 3H), 2.76 (p, J=7.2 Hz, 1H), 2.58 (p, J=6.9 Hz, 1H), 2.44 (p, J=6.9 Hz, 1H), 1.33 (d, J=6.8 Hz, 3H), 1.19 (d, J=6.9 Hz, 3H), 1.14 (d, J=6.9 Hz, 3H), 1.10 (d, J=6.9 Hz, 3H), 0.88 (d, J=7.3 Hz, 3H), 0.81 (d, J=7.3 Hz, 3H) ppm. ¹³C NMR (151 MHz, CDCl₃) δ 158.16, 148.92, 147.49, 145.41, 141.59, 140.23, 138.02, 130.75, 129.31, 128.40, 127.95, 127.09, 126.38, 119.70, 108.54, 77.24, 77.03, 76.82, 76.82, 56.41, 32.36, 30.59, 29.70, 25.09, 24.41, 24.33, 24.26, 23.55, 23.16, 22.81, 20.85 ppm.

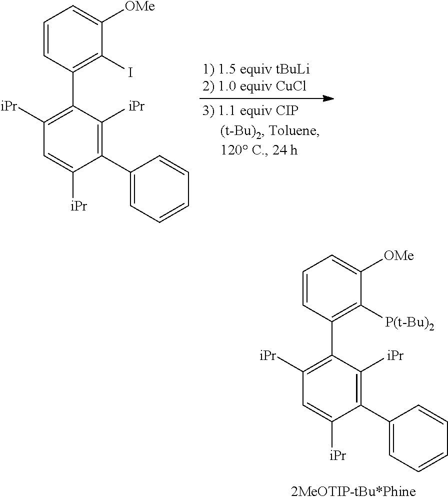

### Di-tert-butyl(2′,4′,6′-triisopropyl-3-methoxy-[1,1′:3′,1″-terphenyl]-2-yl)phosphane (2MeOTIP-tBu*Phine)

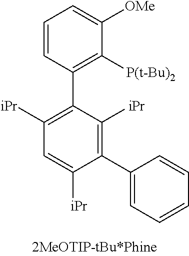

A 10 mL oven-dried Schlenk tube was charged with 2MeOTIP-iodide (256 mg, 0.5 mmol) and anhydrous toluene (1.0 mL). The tube was capped with a rubber septum and was cooled to −78° C. and 1.7 M t-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (108 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (160 mg, 60%), labeled as 2MeOTIP-tBu*Phine. ¹H NMR (400 MHz, Benzene-d₆) δ 7.57 (s, 1H), 7.55-7.42 (m, 2H), 7.25-7.14 (m, 5H), 6.57 (dd, J=7.6, 1.7 Hz, 1H), 3.46-3.36 (m, 1H), 3.36 (s, 3H), 2.94-2.72 (m, 2H), 1.55 (d, J=6.7 Hz, 3H), 1.42 (dd, J=17.9, 12.0 Hz, 18H), 1.33 (dd, J=16.0, 6.8 Hz, 6H), 1.27 (dd, J=6.9, 2.5 Hz, 6H) ppm. ¹³C NMR (151 MHz, Benzene-d₆) δ 161.90, 152.51, 147.08, 142.77, 142.12, 139.30, 137.51, 132.19, 130.77, 128.90, 126.39, 124.86, 119.34 (d, J_{C-P}=54.0 Hz), 112.17, 108.67, 53.11, 34.13, 32.81 (d, J_{C-P}=3.5 Hz), 32.30, 31.56 (dd, J_{C-P}=16.3, 11.8 Hz), 29.50, 26.82, 24.59 (d, J_{C-P}=29.7 Hz), 24.04, 23.93-22.62 (m) ppm. ³¹P NMR (243 MHz, CDCl₃) δ=33.05 ppm.

### Synthesis of Ligand 2MeO-5MeTIP-tBu*Phine

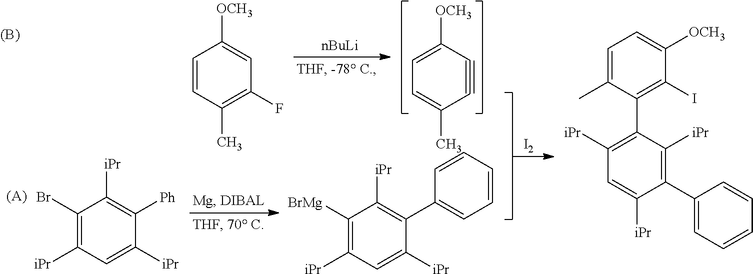

### 2-iodo-2′,4′,6′-triisopropyl-3-methoxy-6-methyl-1,1′:3′,1″-terphenyl

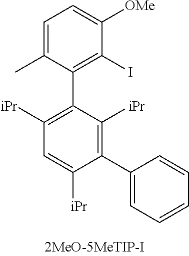 [SMILES: COc1ccc(C)c(-c2c(C(C)C)cc(C(C)C)c(-c3ccccc3)c2C(C)C)c1I]

A 10 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 2-bromo-1,3,5-triisopropylbenzene (566 mg, 2 mmol) was added potion-by-potion with vigorous stirring. The tube was capped with a rubber septum and was heated to 70° C. for 2 h (A). In another 10 mL oven-dried Schlenk tube was charged with 2-fluoro-4-methoxy-1-methylbenzene (140 mg, 1 mmol) and anhydrous THF (3.5 mL). The tube was cooled to −78° C. and 1.6 M n-BuLi solution in hexane (0.64 mL, 1 mmol) was added dropwise over 10 min under vigorous stirring. The mixture was stirred at −78° C. for 40 min (B). A was transferred into to B by syringe over 15 min at −78° C. An additional 1 mL of THF was used to rinse the reaction and was also transferred to B. The combined reaction mixture was stirred at −78° C. for another 1 h and then slowly warmed to room temperature and vigorously stirred for overnight. At this time, the mixture was cooled to 0° C. using ice bath, and iodine (762 mg, 3 mmol) in 4 mL anhydrous THF was added drop-wise over 5 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (10 mL×3) and the combined organic phases were dried over anhydrous MgSO₄. After concentration, the crude product was purified by silica gel chromatography (EtOAc/petroleum ether=1/20) followed by crystallization in methanol to get a white solid (185 mg, 34%), labeled as 2MeO-SMeTIP-I. ¹H NMR (600 MHz, Chloroform-d) δ 7.46-7.29 (m, 4H), 7.25 (dd, J=8.3, 0.8 Hz, 2H), 7.12 (s, 1H), 6.78 (d, J=8.3 Hz, 1H), 3.97 (s, 3H), 2.73-2.59 (m, 1H), 2.54 (p, J=6.8 Hz, 1H), 2.37 (p, J=6.8 Hz, 1H), 2.13 (s, 3H), 1.37 (d, J=7.0 Hz, 3H), 1.28 (d, J=6.8 Hz, 3H), 1.18-1.07 (m, 6H), 0.88 (d, J=7.2 Hz, 3H), 0.82 (d, J=7.2 Hz, 3H) ppm. ¹³C NMR (151 MHz, Chloroform-d) δ 156.49, 147.77, 146.49, 144.69, 141.28 (d, J=23.7 Hz), 139.64, 138.22, 131.97, 131.47, 130.62, 130.17, 129.81, 127.88, 126.87 (d, J=11.2 Hz), 126.38, 120.38 (d, J=35.2 Hz), 109.03, 56.39, 34.27, 32.38, 30.78, 30.26, 29.50, 24.63 (d, J=5.6 Hz), 24.47, 24.29, 24.22, 24.11, 23.42 (d, J=11.7 Hz), 21.54 ppm.

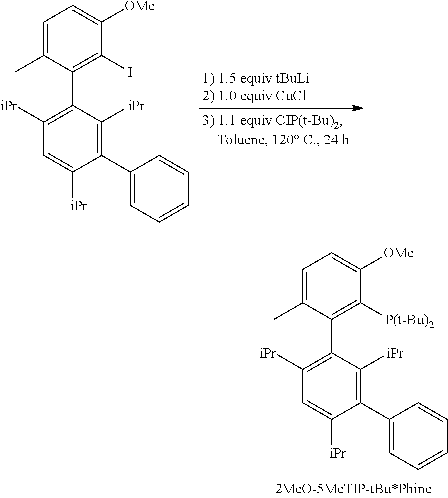

### Di-tert-butyl(2(2′,4′,6′-triisopropyl-3-methoxy-6-methyl-[1,1′:3′,1″-terphenyl]-2-yl)phosphane

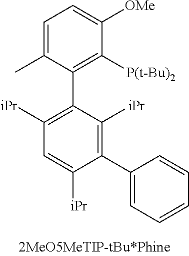

A 10 mL oven-dried Schlenk tube was charged with 2MeO-5MeTIP-iodide (263 mg, 0.5 mmol) and anhydrous toluene (1.0 mL). The tube was capped with a rubber septum and was cooled to −78° C. and 1.7 M t-BuLi solution in petane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (108 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (171 mg, 63%), labeled as 2MeO-5MeTIP-tBu*Phine. ¹H NMR (400 MHz, Benzene-d₆) δ 7.64 (s, 1H), 7.57-7.51 (m, 2H), 7.36-7.27 (m, 4H), 6.68 (d, J=8.4 Hz, 1H), 3.46 (s, 3H), 3.43-3.28 (m, 1H), 2.90 (p, J=6.8 Hz, 2H), 2.22 (s, 3H), 1.64 (d, J=6.7 Hz, 3H), 1.52 (dd, J=11.9, 6.2 Hz, 18H), 1.39-1.28 (m, 12H), 1.25 (d, J=7.2 Hz, 3H) ppm. ¹³C NMR (151 MHz, Benzene-d₆) δ 160.48, 151.94 (d, J_{CP}=38.4 Hz), 147.39, 145.95, 142.14 (d, J_{CP}=25.8 Hz), 138.32, 132.35, 131.54, 130.59 (d, J_{CP}=8.2 Hz), 127.02 (d, J_{CP}=2.8 Hz), 126.52, 125.09 (d, J_{CP}=46.5 Hz), 120.12, 108.60, 53.11, 34.32 (d, J_{CP}=29.3 Hz), 33.86 (d, J_{CP}=30.3 Hz), 32.98 (d, J_{CP}=3.7 Hz), 32.46-31.32 (m), 29.60, 25.57, 25.30 (d, J_{CP}=3.3 Hz), 24.56, 24.19, 23.79, 22.52 (d, J=2.5 Hz) ppm. ³¹P NMR (243 MHz, CDCl₃) δ=35.90 ppm.

### Synthesis of Ligand 25MeOTIP-tBu*Phine

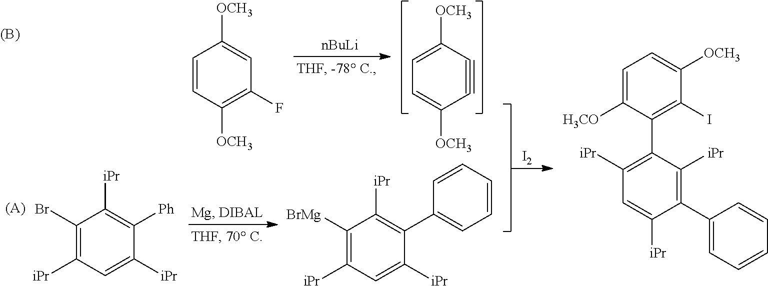

### 2-iodo-2′,4′,6′-triisopropyl-3,6-dimethoxy-1,1′:3′,1″-terphenyl

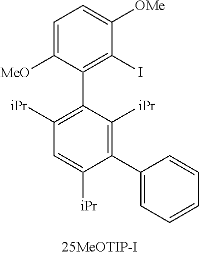 [SMILES: COc1ccc(OC)c(-c2c(C(C)C)cc(C(C)C)c(-c3ccccc3)c2C(C)C)c1I]

A 10 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 2-bromo-1,3,5-triisopropylbenzene (566 mg, 2 mmol) was added potion-by-potion with vigorous stirring. The tube was capped with a rubber septum and was heated to 70° C. for 2 h (A). In another 10 mL oven-dried Schlenk tube was charged with 2-fluoro-1,4-dimethoxybenzene (156 mg, 1 mmol) and anhydrous THF (3.5 mL). The tube was cooled to −78° C. and 1.6 M n-BuLi solution in hexane (0.64 mL, 1 mmol) was added dropwise over 10 min under vigorous stirring. The mixture was stirred at −78° C. for 40 min (B). A was transferred into to B by syringe over 15 min at −78° C. An additional 1 mL of THF was used to rinse the reaction and was also transferred to B. The combined reaction mixture was stirred at −78° C. for another 1 h and then slowly warmed to room temperature and vigorously stirred for overnight. At this time, the mixture was cooled to 0° C. using ice bath, and iodine (762 mg, 3 mmol) in 4 mL anhydrous THF was added drop-wise over 5 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (10 mL×3) and the combined organic phases were dried over anhydrous MgSO₄. After concentration, the crude product was purified by silica gel chromatography (EtOAc/petroleum ether=1/20) followed by crystallization in methanol to get a white solid (162 mg, 30%), labeled as 25MeOTIP-I. ¹H NMR (600 MHz, Chloroform-d) δ 7.39-7.29 (m, 4H), 7.28-7.24 (m, 1H), 7.21 (s, 1H), 6.90 (d, J=8.9 Hz, 1H), 6.82 (d, J=8.9 Hz, 1H), 3.91 (s, 3H), 3.71 (s, 3H), 2.66 (p, J=7.2 Hz, 1H), 2.55-2.45 (m, 1H), 2.34 (p, J=6.8 Hz, 1H), 1.23 (d, J=6.9 Hz, 3H), 1.11 (dd, J=12.6, 6.9 Hz, 6H), 1.04 (d, J=6.8 Hz, 3H), 0.81 (d, J=7.2 Hz, 3H), 0.72 (d, J=7.2 Hz, 3H) ppm. ¹³C NMR (151 MHz, Chloroform-d) δ 152.42 (d, J=24.3 Hz), 147.34, 145.33, 141.78, 137.81, 137.38, 137.03, 131.88, 131.03, 126.95, 126.24, 119.68, 109.92, 109.16, 56.81, 55.32, 32.61, 30.91, 29.66, 24.68 (d, J=2.3 Hz), 24.06, 23.75, 22.91 (d, J=21.4 Hz) ppm.

### Alternative Route for Synthesis of 25MeOTIP-tBu*Phine

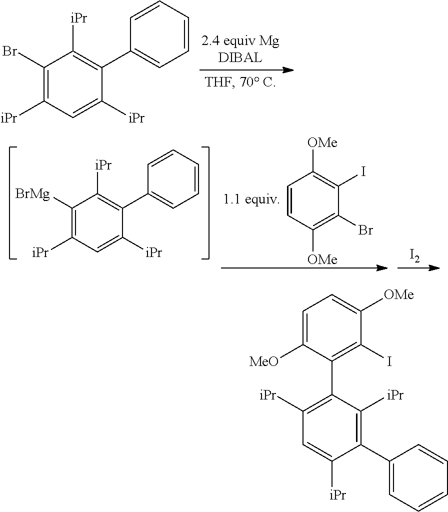

2-iodo-2′,4′,6′-triisopropyl-3,6-dimethoxy-1,1′:3′,1″-terphenyl. A 20 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 2-bromo-1,3,5-triisopropylbenzene (566 mg, 2 mmol) was added potion-by-potion with vigorous stirring. The tube was capped with a rubber septum and was heated to 70° C. for 2 h. 1-bromo-2-iodo-3,6-dimethoxybenzene (700 mg, 2.1 mmol) in anhydrous THF (3 mL) was injected through syringe. An additional 1 mL of THF was used to rinse the reaction and was also transferred into the mixture. The combined reaction mixture was stirred at 70° C. overnight. At this time, the mixture was cooled to 0° C. using ice bath, and iodine (1.3 g, 5 mmol) in 6 mL anhydrous THF was added drop-wise over 10 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (20 mL×3) and the combined organic phases were dried over anhydrous MgSO₄. After concentration, the crude product was purified by silica gel chromatography (EtOAc/petroleum ether=1/20) followed by crystallization in methanol to get a white solid (415 mg, 40%), labeled as 25MeOTIP-I.

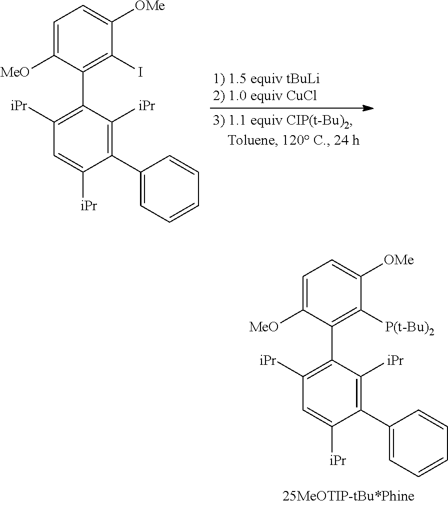

### Di-tert-butyl(2′,4′,6′-triisopropyl-3,6-dimethoxy-[1,1′:3′,1″-terphenyl]-2-yl)phosphane (25MeOTIP-tBu*Phine)

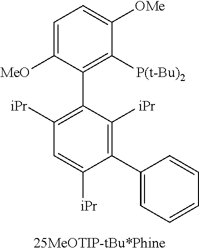

A 10 mL oven-dried Schlenk tube was charged with 25MeOTIP-iodide (263 mg, 0.5 mmol) and anhydrous toluene (1.0 mL). The tube was capped with a rubber septum and was cooled to −78° C. and 1.7 M t-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (108 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled to room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (157 mg, 56%), labeled as 25MeOTIP-tBu*Phine. ¹H NMR (600 MHz, Chloroform-d) δ 7.36-7.29 (m, 3H), 7.27 (dd, J=4.7, 2.4 Hz, 1H), 7.10 (s, 1H), 6.87 (d, J=8.9 Hz, 1H), 6.81 (d, J=8.9 Hz, 1H), 3.78 (s, 3H), 3.61 (s, 3H), 2.74 (p, J=7.0 Hz, 1H), 2.43 (dp, J=35.3, 6.8 Hz, 2H), 1.22 (d, J=6.6 Hz, 4H), 1.13 (dd, J=12.0, 2.8 Hz, 18H), 1.09 (dd, J=9.0, 6.8 Hz, 6H), 0.97 (d, J=6.6 Hz, 3H), 0.90 (d, J=7.0 Hz, 3H) ppm. ¹³C NMR (151 MHz, Chloroform-d) δ 155.84, 152.34 (d, J_{CP}=11.5 Hz), 146.30 (d, J_{CP}=17.8 Hz), 142.38, 141.18 (d, J_{CP}=37.5 Hz), 136.60, 133.95 (d, J_{CP}=7.1 Hz), 132.44, 131.02, 126.65 (d, J_{CP}=23.1 Hz), 125.97, 118.64, 110.60, 107.82, 53.98, 53.79, 33.97, 33.48, 32.79 (d, J_{CP}=2.2 Hz), 31.73 (dd, J_{CP}=16.6, 3.9 Hz), 31.23 (d, J_{CP}=2.0 Hz), 29.35, 25.54, 24.71, 24.14 (d, J_{CP}=14.5 Hz), 23.86, 23.49 ppm. ³¹P NMR (243 MHz, CDCl₃) S=34.78 ppm.

### Synthesis of Ligand 25MeOTIP-Ad*Phine

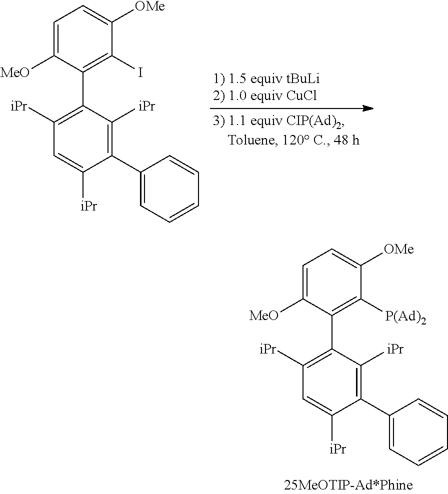

### Di((1s,3R,5R,7S)-adamantan-1-yl)(2′,4′,6′-triisopropyl-3,6-dimethoxy-[1,1′:3,1″-terphenyl]-2-yl)phosphane (25MeOTIP-Ad*Phine)

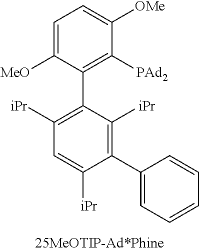

A 10 mL oven-dried Schlenk tube was charged with 25MeOTIP-iodide (263 mg, 0.5 mmol) and anhydrous toluene (1.0 mL). The tube was capped with a rubber septum and was cooled to −78° C. and 1.7 M t-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-adamantylchlorophosphine (202 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 48 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (240 mg, 67%), labeled as 25MeOTIP-Ad*Phine. ¹H NMR (400 MHz, Benzene-d₆) δ 7.71 (d, J=7.6 Hz, 1H), 7.69-7.62 (m, 2H), 7.56-7.36 (m, 3H), 7.16 (s, 1H), 6.76 (d, J=8.9 Hz, 1H), 6.69 (d, J=8.9 Hz, 1H), 3.59 (s, 3H), 3.47 (d, J=7.2 Hz, 1H), 3.36 (s, 3H), 3.06-2.81 (m, 2H), 2.35 (d, J=25.9 Hz, 10H), 2.15 (s, 7H), 2.00-1.85 (m, 10H), 1.73 (d, J=6.8 Hz, 3H), 1.53 (d, J=7.1 Hz, 3H), 1.47 (d, J=6.6 Hz, 3H), 1.37 (d, J=6.8 Hz, 3H), 1.32 (d, J=7.0 Hz, 3H), 1.21 (d, J=7.2 Hz, 3H) ppm. ¹³C NMR (151 MHz, Benzene-d₆) δ 152.89 (d, J=11.2 Hz), 147.15 (d, J_{CP}=16.5 Hz), 143.42, 142.80, 142.16 (d, J_{CP}=38.3 Hz), 137.63, 134.99 (d, J_{CP}=6.6 Hz), 132.72, 131.29, 127.31 (d, J_{CP}=9.7 Hz), 126.59, 125.37, 119.31, 113.24, 111.19, 108.31, 55.17, 53.69, 42.50 (t, J_{CP}=14.6 Hz), 40.15-38.51 (m), 37.48 (d, J_{CP}=6.8 Hz), 33.60, 31.88, 29.77 (dd, J_{CP}=23.9, 9.0 Hz), 26.23, 25.23, 24.96-24.06 (m), 24.32, 24.16 ppm. ³¹P NMR (243 MHz, CDCl₃) δ=37.08 ppm.

### Synthesis of Ligand 25MeOTIP-Cy*Phine

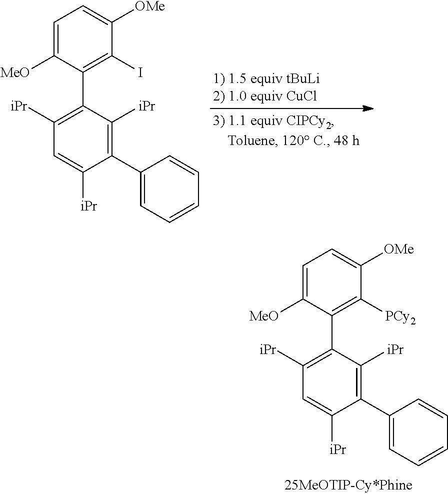

### Dicyclohexyl(2′,4′,6′-triisopropyl-3,6-dimethoxy-[1,1′:3′,1″-terphenyl]-2-yl)phosphane (25MeOTIP-Cy*Phine)

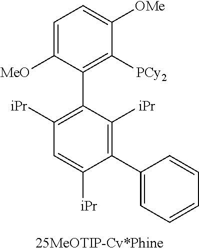

A 10 mL oven-dried Schlenk tube was charged with 25MeOTIP-iodide (263 mg, 0.5 mmol) and anhydrous toluene (1.0 mL). The tube was capped with a rubber septum and was cooled to −78° C. and 1.7 M t-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, PCy₂Cl (134 mg, 0.6 mmol, 1.1 equiv.) in anhydrous toluene (1 mL) was injected slowly through syringe into the above mixture at −78° C. The combined reaction mixture was stirred vigorously at room temperature for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (240 mg, 67%), labeled as 25MeOTIP-Cy*Phine. ¹H NMR (600 MHz, Chloroform-d) δ 7.33 (dddd, J=11.6, 9.5, 6.5, 3.6 Hz, 5H), 7.14 (s, 1H), 6.87 (d, J=8.9 Hz, 1H), 6.80 (d, J=8.6 Hz, 1H), 3.84 (s, 3H), 3.62 (s, 3H), 2.71 (p, J=7.2 Hz, 1H), 2.49 (p, J=6.9 Hz, 1H), 2.38 (p, J=7.0 Hz, 1H), 1.94-1.84 (m, 2H), 1.79-1.54 (m, 6H), 1.49-1.37 (m, 2H), 1.27 (d, J=6.9 Hz, 14H), 1.14 (d, J=6.8 Hz, 14H), 1.08 (d, J=6.9 Hz, 3H), 0.97 (d, J=6.9 Hz, 3H), 0.88 (d, J=7.1 Hz, 3H), 0.59 (d, J=7.2 Hz, 3H) ppm. ¹³C NMR (151 MHz, CDCl₃) δ 156.26, 156.24, 152.19, 152.12, 146.33, 145.64, 142.41, 142.21, 140.17, 139.93, 136.87, 134.04, 133.98, 132.41, 131.36, 126.68, 126.60, 126.40, 126.04, 118.76, 110.62, 108.46, 55.02, 54.37, 36.19, 36.17, 36.09, 36.07, 32.67, 32.65, 32.53, 32.51, 32.20, 31.20, 31.16, 31.12, 31.07, 30.76, 29.38, 27.99, 27.93, 27.91, 27.85, 27.60, 27.51, 26.54, 26.44, 25.09, 24.48, 24.44, 24.40, 23.82, 23.57, 23.36 ppm. ³¹P NMR (243 MHz, CDCl₃) δ=−1.77 ppm.

### Synthesis of Ligand 25MeOTIP-tBu*Phine-CF3

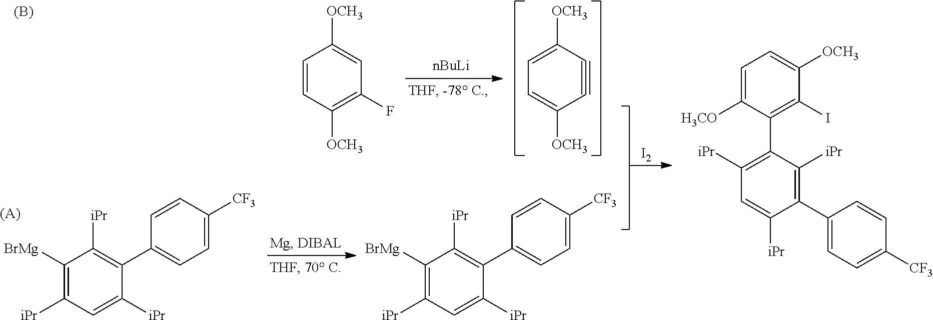

### 2-iodo-2′,4′,6′-triisopropyl-3,6-dimethoxy-4″-(trifluoro-methyl)-1,1′:3′,1″-terphenyl (25MeOTIP-I—CF3)

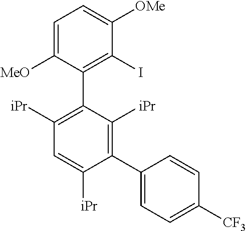 [SMILES: COc1ccc(OC)c(-c2c(C(C)C)cc(C(C)C)c(-c3ccc(C(F)(F)F)cc3)c2C(C)C)c1I]

A 10 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 3-bromo-2,4,6-triisopropyl-4′-(trifluoromethyl)-1,1′-biphenyl (852 mg, 2 mmol) was added potion-by-potion with vigorous stirring. The tube was capped with a rubber septum and was heated to 70° C. for 2 h (A). In another 10 mL oven-dried Schlenk tube was charged with 2-fluoro-1,4-dimethoxybenzene (156 mg, 1 mmol) and anhydrous THF (3.5 mL). The tube was cooled to −78° C. and 1.6 M n-BuLi solution in hexane (0.64 mL, 1 mmol) was added dropwise over 10 min under vigorous stirring. The mixture was stirred at −78° C. for 40 min (B). A was transferred into to B by syringe over 15 min at −78° C. An additional 1 mL of THF was used to rinse the reaction and was also transferred to B. The combined reaction mixture was stirred at −78° C. for another 1 h and then slowly warmed to room temperature and vigorously stirred for overnight. At this time, the mixture was cooled to 0° C. using ice bath, and iodine (762 mg, 3 mmol) in 4 mL anhydrous THF was added drop-wise over 5 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (10 mL×3) and the combined organic phases were dried over anhydrous MgSO₄. After concentration, the crude product was purified by silica gel chromatography (EtOAc/petroleum ether=1/20) followed by crystallization in methanol to get a white solid (226 mg, 37%), labeled as 25MeOTIP-I. ¹H NMR (600 MHz, Chloroform-d) δ 7.66-7.61 (m, 3H), 7.46-7.39 (m, 2H), 7.23 (s, 1H), 6.90 (d, J=8.9 Hz, 1H), 6.82 (d, J=8.9 Hz, 1H), 3.91 (s, 3H), 3.71 (s, 3H), 2.65 (p, J=7.2 Hz, 1H), 2.37 (dp, J=25.3, 6.9 Hz, 2H), 1.23 (d, J=6.8 Hz, 3H), 1.11 (dd, J=10.3, 6.9 Hz, 6H), 1.04 (d, J=6.9 Hz, 3H), 0.80 (d, J=7.2 Hz, 3H), 0.71 (d, J=7.2 Hz, 3H) ppm. ¹³C NMR (151 MHz, Chloroform-d) δ 152.40 (d, J=40.7 Hz), 147.01, 146.10, 145.85, 141.68, 137.11 (d, J=48.9 Hz), 136.32, 132.12, 131.30, 128.68 (d, J=32.3 Hz), 124.02, 119.94, 110.00, 109.30, 56.81, 55.31, 32.51, 30.93, 29.72, 24.61 (d, J=4.2 Hz), 24.00, 23.69, 22.94 (d, J=12.1 Hz) ppm. ¹⁹F NMR (376 MHz, CDCl₃) δ=−62.15 ppm.

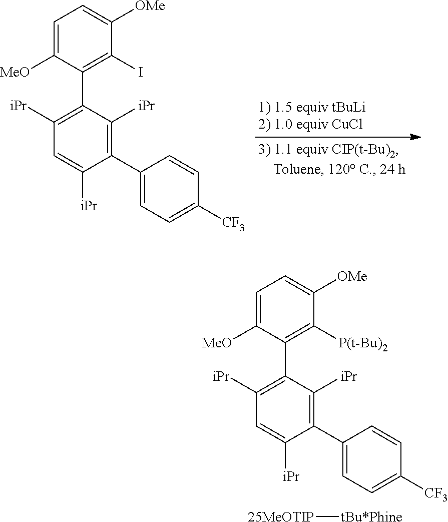

### Di-tert-butyl(2′,4′,6′-triisopropyl-3,6-dimethoxy-4″-(trifluoromethyl)-[1,1′:3′,1″-terphenyl]-2-yl)phosphane (25MeOTIP-tBu*Phine-CF3)

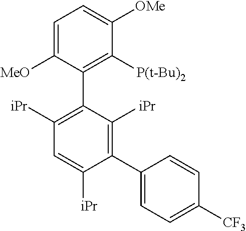

A 10 mL oven-dried Schlenk tube was charged with 25MeOTIP-iodide (263 mg, 0.5 mmol) and anhydrous toluene (1.0 mL). The tube was capped with a rubber septum and was cooled to −78° C. and 1.7 M t-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (108 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (166 mg, 56%), labeled as 25MeOTIP-tBu*Phine-CF3. ¹H NMR (400 MHz, Benzene-d₆) δ 7.63 (s, 1H), 7.57 (dd, J=4.1, 2.1 Hz, 4H), 7.52 (s, 1H), 6.76 (d, J=8.9 Hz, 1H), 6.67 (d, J=9.0 Hz, 1H), 3.48 (s, 3H), 3.36 (s, 3H), 2.90 (p, J=6.7 Hz, 1H), 2.64 (p, J=6.8 Hz, 1H), 1.64 (d, J=6.7 Hz, 3H), 1.51 (dd, J=14.5, 12.0 Hz, 18H), 1.42 (d, J=6.6 Hz, 3H), 1.37 (d, J=7.0 Hz, 3H), 1.29 (d, J=7.0 Hz, 4H), 1.23 (d, J=6.8 Hz, 3H), 0.97 (d, J=7.2 Hz, 3H) ppm. ¹³C NMR (151 MHz, Chloroform-d) δ 155.89, 146.97, 146.00, 142.64, 140.74 (d, J_{CP}=37.8 Hz), 135.12, 132.70, 131.20, 123.68 (dd, J_{CP}=35.5, 3.6 Hz), 119.93, 118.87, 110.71, 110.00, 109.29, 108.01, 53.98, 53.81, 33.74 (dd, J_{CP}=51.1, 29.2 Hz), 32.68 (d, J_{CP}=2.4 Hz), 31.77 (d, J_{CP}=3.5 Hz), 31.66 (d, J_{CP}=3.5 Hz), 29.40, 25.52, 24.62, 24.39, 24.04, 23.90, 23.45 ppm. ³¹P NMR (243 MHz, CDCl₃) δ=34.54 ppm. ¹⁹F NMR (376 MHz, CDCl₃) δ=−61.84 ppm.

### Synthesis of Ligand 25MeOTIP-tBu*Phine-MeO

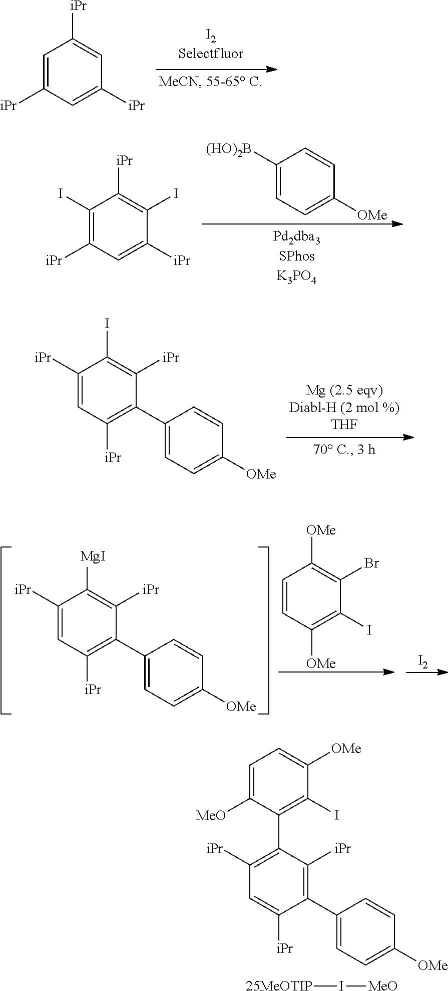

### 2,4-diiodo-1,3,5-triisopropylbenzene

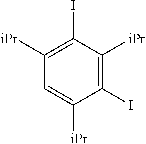 [SMILES: CC(C)c1cc(C(C)C)c(I)c(C(C)C)c1I]

A 250 mL flask wrapped with aluminium foil equipped with a magnetic bar and a reflux condenser. To the flask 2.04 g (10 mmol) of 1,3,5-triisopropylbenzene, 7.62 g (30 mmol) of iodine, 10.62 g (30 mmol) of selectfluor and acetonitrile (70 mL) was added and the mixture will turn dark purple. The reaction was then allowed to stir a 55° C.-65° C. for 20 h. The solvent was removed and dichloromethane was added. The mixture was filtered. The filtrates were then collected and wash with concentrated sodium thiosulfate solution. The organic layer will decolourise to bright orange colour. The organic layer was collected and dry with MgSO₄. The crude was then purified by column with hexane to afford a white solid (0.51 g, 55%). ¹H (600M Hz, CDCl₃) δ: 6.98 (s, 1H), 4.15 (quintet, ³J_{H,H}=7.38 Hz, 1H), 3.48 (quintet, ³J_{H,H}=6.78 Hz, 1H), 3.42 (quintet, ³J_{H,H}=6.78, 1H), 1.50 (s, 3H), 1.49 (s, 3H), 1.24 (d, ³J_{H,H}=4.92 Hz, 6H), 1.23 (d, ³J_{H,H}=4.98 Hz, 6H) ppm. ¹³C (150 MHz, CDCl₃) δ: 151.90, 151.66, 149.04, 121.32, 110.72, 98.54, 45.20, 40.39, 38.75, 23.35, 20.01 ppm. 3-iodo-2,4,6-triisopropyl-4′-methoxy-1,1′-biphenyl.

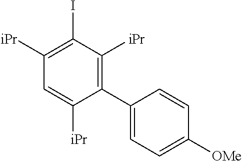 [SMILES: COc1ccc(-c2c(C(C)C)cc(C(C)C)c(I)c2C(C)C)cc1]

To an oven dry 2 neck 50 mL flask equip was a condenser and magnetic stir bar, 2.5 g (5.5 mmol) of 2,4-diiodo-1,3,5-triisopropylbenzene, 1.25 g (8.25 mmol) of (4-methoxyphenyl)boronic acid, 2.33 g (11 mmol) of potassium phosphate tribasic, 51 mg (0.005 mmol) of tris(dibenzylideneacetone)dipalladium(0)₃, 45 mg (0.011 mmol) of SPhos was added. The flask was then vacuum for 5 min and flush with argon after which dry tetrahydrofuran (5.5 mL) and dry toluene (5.5 mL) was added to the flask. The black mixture was then allowed to stir at 100° C. for 16 h. The mixture was filter through silica and wash with ethyl acetate. The reddish filtrate was collect and solvent. The crude was purified by column gradient with hexane and ethyl acetate mixture (10:1) to afford a white solid (0.72 g, 30%). ¹H (600 MHz, CDCl₃) δ; 7.09-7.03 (m, 3H), 6.95-6.90 (m, 2H), 3.88 (s, 3H), 3.52 (quintet, ³J_{H,H}=6.6, 1H), 3.16 (t, ³J_{H,H} 6.52 Hz, 1H), 2.51-0.49 (m, 1H), 1.37 (d, ³J_{H,H}=7.2 Hz, 2H), 1.31 (d, ³J_{H,H}=6.6 Hz, 6H), 1.04 (t, ³J_{H,H}=7.2 Hz, 6H), 0.89 (d, ³J_{H,H}=7.2 Hz, 3H) ppm. ¹³C (150 MHz, CDCl₃) δ: 158.49, 150.74, 150.46, 148.71, 146.71, 138.33, 132.22, 132.01, 129.84, 120.67, 120.52, 113.61, 112.50, 109.99, 55.19, 42.56, 40.39, 38.97, 34.09, 30.47, 29.08, 24.05, 23.95, 23.56, 23.51, 22.79, 20.09 ppm.

### 2-iodo-2′,4′,6′-triisopropyl-3,4″,6-trimethoxy-1,1′:3′,1″-terphenyl

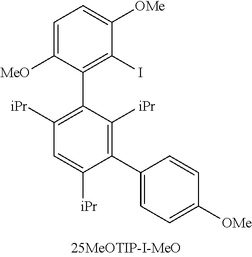 [SMILES: COc1ccc(-c2c(C(C)C)cc(C(C)C)c(-c3c(OC)ccc(OC)c3I)c2C(C)C)cc1]

A 20 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 3-iodo-2,4,6-triisopropyl-4′-methoxy-1,1′-biphenyl (435 mg, 1 mmol, 1.0 equiv.) was added potion-by-potion with vigorous stirring. The tube was capped with a rubber septum and was heated to 70° C. for 3 h. 1-bromo-2-iodo-3,6-dimethoxybenzene (511 mg, 1.5 mmol) in anhydrous THF (3 mL) was injected through syringe. An additional 1 mL of THF was used to rinse the reaction and was also transferred into the mixture. The combined reaction mixture was stirred at 70° C. for 16 h. At this time, the mixture was cooled to 0° C. using ice bath, and iodine (508 mg, 2 mmol, 2.0 equiv.) in 6 mL anhydrous THF was added drop-wise over 10 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (20 mL×3) and the combined organic phases were dried over anhydrous MgSO₄. After concentration, the crude product was purified by silica gel chromatography with gradient of hexane and ethyl acetate to get a white solid (176 mg, 30%), labeled as 25MeOTIP-I-MeO. ¹H NMR (600 MHz, Chloroform-d) δ 7.19-7.18 (m, 2H), 7.16-7.14 (m, 1H), 6.90-6.88 (m, 3H), 6.80 (d, J=9 Hz, 1H), 3.90 (s, 3H), 3.87 (s, 3H), 3.70 (s, 3H), 2.65 (q, J=7.2 Hz, 1H), 2.54 (q, J=6.6 Hz, 1H), 2.32 (q, J=6.6 Hz, 1H), 1.22 (d, J=7.2 Hz, 3H), 1.11 (d, J=6 Hz, 3H), 1.09 (d, J=6 Hz, 3H), 1.08 (d, J=6 Hz, 3H), 0.816 (d, J=7.2 Hz, 3H), 0.746 (d, J=7.2 Hz, 3H) ppm. ¹³C NMR (151 MHz, Chloroform-d) δ 158.07, 152.48, 152.31, 147.87, 145.20, 142.26, 137.44, 137.00, 133.82, 132.78, 131.91, 119.61, 112.35, 112.26, 109.91, 109.11, 56.80, 55.32, 55.15, 32.62, 30.90, 29.60, 24.68, 24.66, 24.04, 23.72, 22.98, 22.89 ppm.

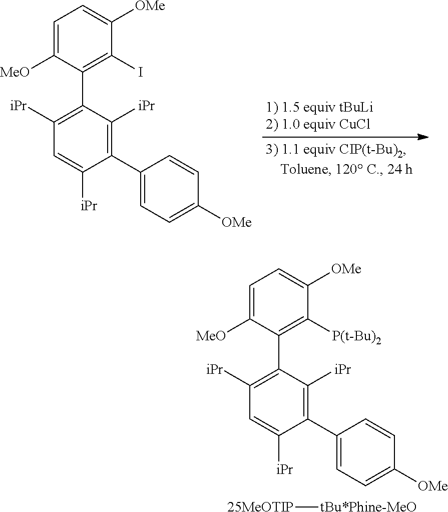

### Di-tert-butyl(2′,4′,6′-triisopropyl-3,4″,6-trimethoxy-[1,1′:3′,1″-terphenyl]-2-yl)phosphane

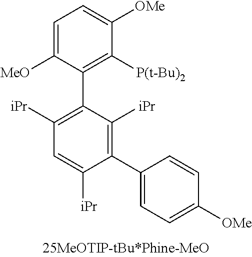

A 10 mL oven-dried Schlenk tube was charged with 25MeOTIP-I-MeO (570 mg, 1.0 mmol, 1.0 equiv) and anhydrous toluene (1.0 mL). The tube was capped with a rubber septum and was cooled to −78° C. and 1.7 M t-BuLi solution in pentane (1.2 mL, 2.0 mmol, 2.0 equiv.) was added dropwise over 10 min under vigorous stirring. The mixture was stirred at −78° C. for 1 h (A). In another 10 mL oven-dried Schlenk tube was charged with CuCl (99 mg, 1.0 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (119 mg, 1.2 mmol, 1.2 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (195 mg, 33%), labeled as 25MeOTIP-tBu*Phine-MeO. ¹H NMR (400 MHz, Benzene-d₆) δ 7.45 (s, 1H), 7.38-7.36 (m, 1H), 7.31-7.29 (m, 1H), 6.82-6.78 (m, 2H), 6.55 (d, J=8.88 Hz, 1H), 6.45 (d, J=8.88 Hz, 1H), 3.31 (s, 3H), 3.27 (s, 3H), 3.21 (q, J=7.08 Hz, 1H), 3.14 (s, 1H), 2.78 (q, J=6.9 Hz, 1H), 2.73 (q, J=6.66 Hz, 1H), 1.47 (d, J=3.48 Hz, 3H), 1.34 (d, J=1-1.94 Hz, 11H), 1.30 (d, J=11.94 Hz, 11H), 1.23 (d, J=6.66 Hz, 3H), 1.17 (d, J=6.9 Hz, 3H), 1.13 (d, J=6.9 Hz, 3H), 1.01 (d, J=7.06 Hz, 3H) ppm. ¹³C NMR (150 MHz, CDCl₃) δ 158.49, 155.97, 152.50, 152.42, 147.46, 146.49, 143.52, 141.48, 141.23, 137.05, 134.54, 134.49, 134.29, 133.31, 131.74, 118.97, 112.75, 112.28, 110.92, 108.13, 54.27, 53.41, 53.19, 34.08, 33.88, 33.65, 33.45, 33.32, 33.30, 31.86, 31.84, 31.73, 31.46, 29.52, 25.80, 24.71 ppm. ³¹P NMR (243 MHz, CDCl₃) δ=34.23 ppm.

### Synthesis of Ligand 25MeOTIP-tBu*Phine-nBu

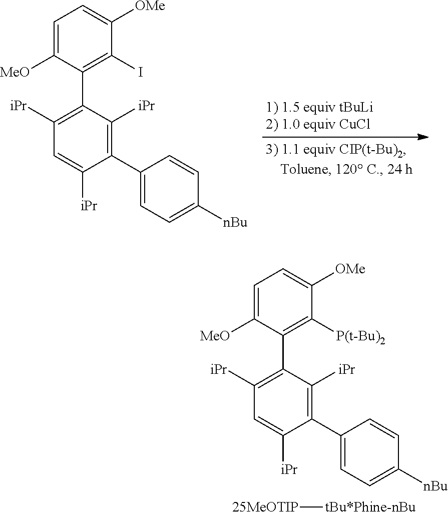

### Di-tert-butyl(4″-butyl-2′,4′,6′-triisopropyl-3,6-dimethoxy-[1,1′:3′,1″-terphenyl]-2-yl)phosphane (25MeOTIP-tBu*Phine-MeO)

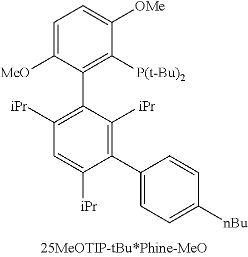

A 10 mL oven-dried Schlenk tube was charged with 25MeOTIP-I-nBu (4″-butyl-2-iodo-2′,4′,6′-triisopropyl-3,6-dimethoxy-1,1′:3′,1″-terphenyl) (550 mg, 1.0 mmol, 1.0 equiv) and anhydrous toluene (1.0 mL). The tube was capped with a rubber septum and was cooled to −78° C. and 1.7 M t-BuLi solution in pentane (1.2 mL, 2.0 mmol, 2.0 equiv.) was added dropwise over 10 min under vigorous stirring. The mixture was stirred at −78° C. for 1 h (A). In another 10 mL oven-dried Schlenk tube was charged with CuCl (99 mg, 1.0 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (119 mg, 1.2 mmol, 1.2 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (265 mg, 43%), labeled as 25MeOTIP-tBu*Phine-nBu. ¹H NMR (400 MHz, Benzene-d₆) δ 7.45-7.43 (m, 2H), 7.38-7.37 (m, 1H), 7.08-7.06 (m, 2H), 6.54 (d, J=8.88 Hz, 1H), 6.45 (d, J=8.94 Hz, 1H), 3.26 (s, 3H), 3.23-3.19 (m, 1H), 3.14 (s, 3H), 2.78 (q, J=6.84 Hz, 1H), 2.73 (q, J=6.66 Hz, 1H), 2.49 (t, J=7.8 Hz, 2H), 1.50-1.46 (m, 5H), 1.32 (d, J=11.8 Hz, 9H), 1.29 (d, J=11.8 Hz, 9H), 1.23 (d, J=6.45 Hz, 4H), 1.17 (d, J=6.9 Hz, 3H), 1.11 (d, J=6.9 Hz, 3H), 0.98 (d, J=7.14 Hz, 3H), 0.82 (t, J=7.32 Hz, 3H) ppm. ¹³C NMR (150 MHz, CDCl₃) δ 155.96, 155.95, 152.50, 152.42, 147.12, 146.48, 143.24, 141.46, 141.21, 140.65, 139.58, 137.37, 134.53, 134.48, 134.30, 132.30, 130.84, 118.99, 110.92, 108.14, 53.41, 53.19, 35.41, 34.08, 33.89, 33.65, 33.45, 33.31, 33.30, 31.86, 31.84, 31.75, 31.73, 31.45, 29.45, 25.80, 24.61, 24.42, 24.27, 24.06, 23.71, 22.37, 13.80 ppm. ³¹P NMR (243 MHz, CDCl₃) δ=34.22 ppm.

### Synthesis of Ligand 24MeOTIP-tBu*Phine

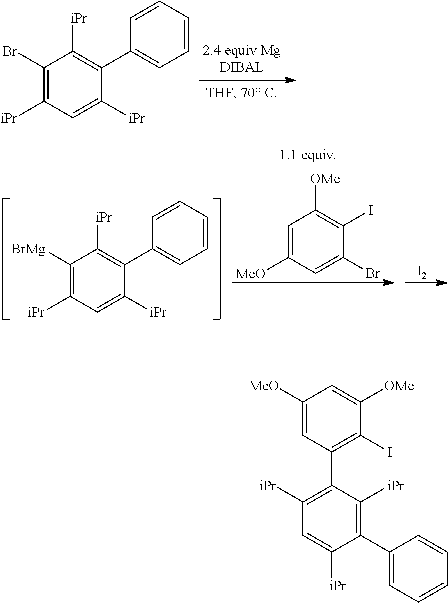

### 2-iodo-2′,4′,6′-triisopropyl-3,5-dimethoxy-1,1′:3′,1″-terphenyl (24MeOTIP-I)

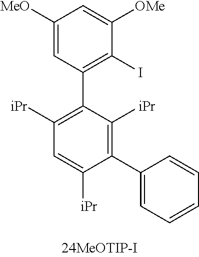 [SMILES: COc1cc(OC)c(I)c(-c2c(C(C)C)cc(C(C)C)c(-c3ccccc3)c2C(C)C)c1]

A 20 mL oven-dried Schlenk tube was charged with magnesium (57.6 mg, 2.4 mmol, 1.2 equiv.) and anhydrous THF (1.5 mL), 5 drops of diisobutylaluminium hydride (DIBAL) was added and stirred for 5 min to activate magnesium. 2-bromo-1,3,5-triisopropylbenzene (566 mg, 2 mmol, 1.0 equiv.) was added potion-by-potion with vigorous stirring. The tube was capped with a rubber septum and was heated to 70° C. for 2 h. 1-bromo-2-iodo-3,5-dimethoxybenzene (700 mg, 2.1 mmol, 1.1 equiv.) in anhydrous THF (3 mL) was injected through syringe. An additional 1 mL of THF was used to rinse the reaction and was also transferred into the mixture. The combined reaction mixture was stirred at 70° C. overnight. At this time, the mixture was cooled to 0° C. using ice bath, and iodine (1.3 g, 5 mmol) in 6 mL anhydrous THF was added drop-wise over 10 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (20 mL×3) and the combined organic phases were dried over anhydrous MgSO₄. After concentration, the crude product was purified by silica gel chromatography (EtOAc/petroleum ether=1/20) followed by crystallization in methanol to get a white solid (401 mg, 37%), labeled as 24MeOTIP-I. ¹H NMR (600 MHz, Chloroform-d) δ 7.29 (s, 2H), 7.07 (s, 2H), 6.46 (d, J=2.7 Hz, 2H), 6.44 (d, J=2.6 Hz, 2H), 3.94 (s, 1H), 3.80 (s, 1H), 2.98 (p, J=6.8 Hz, 1H), 2.48 (p, J=6.8 Hz, 2H), 1.33 (d, J=7.0 Hz, 6H), 1.23 (d, J=6.9 Hz, 6H), 1.07 (d, J=6.8 Hz, 6H) ppm. ¹³C NMR (151 MHz, Chloroform-d) δ 160.54, 158.87, 148.18 (d, J=63.2 Hz), 145.49, 139.35, 120.79, 107.34, 97.19, 83.18, 56.36, 55.58, 34.18, 30.57, 25.20, 24.08, 23.52 ppm.

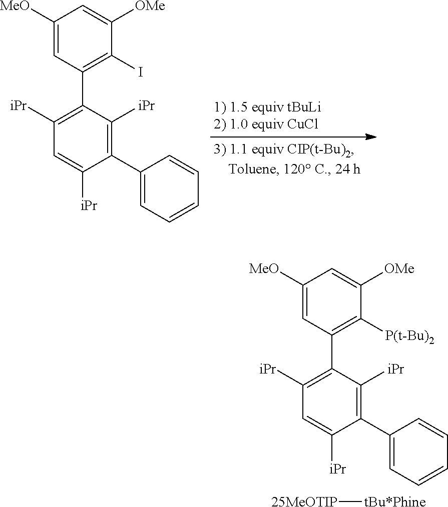

### Di-tert-butyl(2′,4′,6′-triisopropyl-3,5-dimethoxy-[1′,1′:3′,1″-terphenyl]-2-yl)phosphane (24MeOTIP-tBu*Phine)

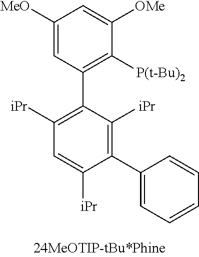

A 10 mL oven-dried Schlenk tube was charged with 24MeOTIP-iodide (271 mg, 0.5 mmol) and anhydrous toluene (1.0 mL). The tube was capped with a rubber septum and was cooled to −78° C. and 1.7 M t-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (108 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (187 mg, 67%), labeled as 24MeOTIP-tBu*Phine. ¹H NMR (400 MHz, Benzene-d₆) δ 7.66 (s, 1H), 7.65-7.59 (m, 1H), 7.56 (d, J=7.2 Hz, 1H), 7.43-7.37 (m, 2H), 7.34-7.27 (m, 2H), 6.86 (d, J=2.3 Hz, 1H), 6.82 (t, J=2.8 Hz, 1H), 6.56 (d, J=2.5 Hz, 1H), 3.57 (s, 3H), 3.40 (s, 3H), 3.05 (p, J=6.8 Hz, 1H), 2.93 (p, J=6.7 Hz, 1H), 1.66 (d, J=6.8 Hz, 3H), 1.52 (dd, J=19.0, 11.9 Hz, 18H), 1.46 (d, J=7.0 Hz, 3H), 1.42-1.27 (m, 9H), 1.12 (d, J=7.2 Hz, 3H) ppm. ¹³C NMR (151 MHz, Benzene-d₆) δ 163.28 (d, J_{CP}=2.5 Hz), 153.47, 147.21, 146.16, 142.23, 139.91, 137.72, 132.38, 130.84, 127.19, 126.55, 119.41, 110.24 (d, J_{CP}=8.0 Hz), 97.41, 54.54, 53.21, 34.08 (d, J_{CP}=27.4 Hz), 33.53 (d, J_{CP}=28.5 Hz), 32.88 (d, J_{CP}=3.7 Hz), 31.76 (t, J_{CP}=16.8 Hz), 31.35, 29.63, 27.24, 25.06, 24.67, 24.11, 23.95, 23.12 ppm. ³¹P NMR (243 MHz, CDCl₃) δ=30.63 ppm.

### Synthesis of Ligand 24MeOTIP-Ad*Phine

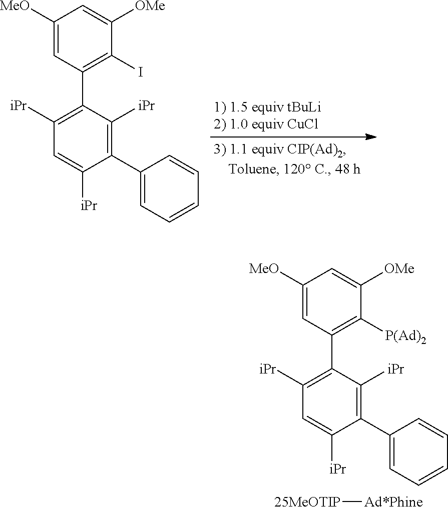

### Di((1s,3R,5R,7S)-adamantan-1-yl)(2′,4′,6′-triisopropyl-3,5-dimethoxy-[1,1′:3′,1″-terphenyl]-2-yl)phosphane (24MeOTIP-Ad*Phine)

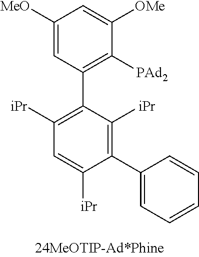

A 10 mL oven-dried Schlenk tube was charged with 24MeOTIP-iodide (263 mg, 0.5 mmol) and anhydrous toluene (1.0 mL). The tube was capped with a rubber septum and was cooled to −78° C. and 1.7 M t-BuLi solution in petane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-adamantylchlorophosphine (202 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 48 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (240 mg, 67%), labeled as 24MeOTIP-Ad*Phine. ¹H NMR (400 MHz, Benzene-d₆) δ 7.71 (d, J=7.6 Hz, 1H), 7.69-7.62 (m, 2H), 7.56-7.36 (m, 3H), 7.16 (s, 1H), 6.76 (d, J=8.9 Hz, 1H), 6.69 (d, J=8.9 Hz, 1H), 3.59 (s, 3H), 3.47 (d, J=7.2 Hz, 1H), 3.36 (s, 3H), 3.06-2.81 (m, 2H), 2.35 (d, J=25.9 Hz, 10H), 2.15 (s, 7H), 2.00-1.85 (m, 10H), 1.73 (d, J=6.8 Hz, 3H), 1.53 (d, J=7.1 Hz, 3H), 1.47 (d, J=6.6 Hz, 3H), 1.37 (d, J=6.8 Hz, 3H), 1.32 (d, J=7.0 Hz, 3H), 1.21 (d, J=7.2 Hz, 3H) ppm. ¹³C NMR (151 MHz, Benzene-d₆) δ 152.89 (d, J=11.2 Hz), 147.15 (d, J_{CP}=16.5 Hz), 143.42, 142.80, 142.16 (d, J_{CP}=38.3 Hz), 137.63, 134.99 (d, J_{CP}=6.6 Hz), 132.72, 131.29, 127.31 (d, J_{CP}=9.7 Hz), 126.59, 125.37, 119.31, 113.24, 111.19, 108.31, 55.17, 53.69, 42.50 (t, J_{CP}=14.6 Hz), 40.15-38.51 (m), 37.48 (d, J_{CP}=6.8 Hz), 33.60, 31.88, 29.77 (dd, J_{CP}=23.9, 9.0 Hz), 26.23, 25.23, 24.96-24.06 (m), 24.32, 24.16 ppm. ³¹P NMR (243 MHz, CDCl₃) δ=37.08 ppm.

### Synthesis of Ligand 23MeOTIP-tBu*Phine

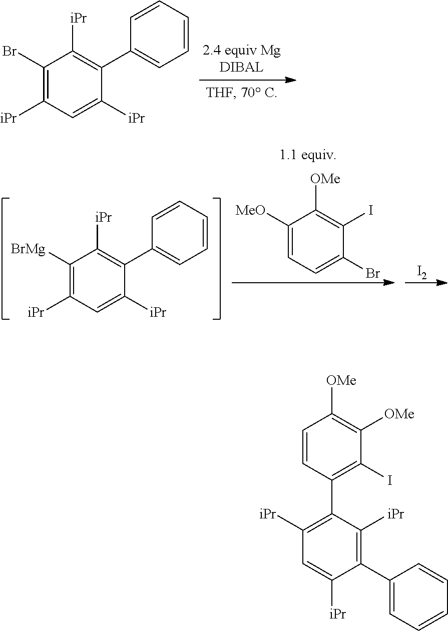

### 2-iodo-2′,4′,6′-triisopropyl-3,4-dimethoxy-1,1′:3′,1″-terphenyl (23MeOTIP-I)

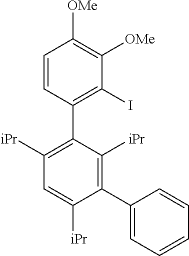 [SMILES: COc1ccc(-c2c(C(C)C)cc(C(C)C)c(-c3ccccc3)c2C(C)C)c(I)c1OC]

A 20 mL oven-dried Schlenk tube was charged with magnesium (57.6 mg, 2.4 mmol) and anhydrous THF (1.5 mL), 5 drops of diisobutylaluminium hydride (DIBAL) was added and stirred for 5 min to activate magnesium. 2-bromo-1,3,5-triisopropylbenzene (566 mg, 2 mmol) was added potion-by-potion with vigorous stirring. The tube was capped with a rubber septum and was heated to 70° C. for 2 h. 1-bromo-2-iodo-3,4-dimethoxybenzene (700 mg, 2.1 mmol) in anhydrous THF (3 mL) was injected through syringe. An additional 1 mL of THF was used to rinse the reaction and was also transferred into the mixture. The combined reaction mixture was stirred at 70° C. overnight. At this time, the mixture was cooled to 0° C. using ice bath, and iodine (1.3 g, 5 mmol) in 6 mL anhydrous THF was added drop-wise over 10 min, then the reaction mixture was stirred for 30 min, and then warmed to stir at room temperature for 2 h. Then, the mixture was quenched with saturated Na₂S₂O₃ (aq.) solution until the red color of bromine disappeared. The aqueous phase was extracted with Et₂O (20 mL×3) and the combined organic phases were dried over anhydrous MgSO₄. After concentration, the crude product was purified by silica gel chromatography (EtOAc/petroleum ether=1/20) followed by crystallization in methanol to get a white solid (415 mg, 40%), labeled as 23MeOTIP-I. ¹H NMR (600 MHz, Chloroform-d) δ 7.41-7.31 (m, 4H), 7.27-7.23 (m, 1H), 7.20 (s, 1H), 7.00 (d, J=8.3 Hz, 1H), 6.94 (d, J=8.3 Hz, 1H), 3.94 (s, 3H), 3.90 (s, 3H), 2.76-2.66 (m, 1H), 2.51 (p, J=6.9 Hz, 1H), 2.38 (p, J=6.9 Hz, 1H), 1.26 (d, J=6.9 Hz, 3H), 1.13 (d, J=6.9 Hz, 6H), 1.08 (d, J=6.9 Hz, 3H), 1.04 (d, J=6.8 Hz, 3H), 0.80 (d, J=7.3 Hz, 3H), 0.76 (d, J=7.2 Hz, 3H) ppm. ¹³C NMR (151 MHz, CDCl₃) δ 150.79, 148.74, 144.81, 143.30, 142.70, 142.16, 137.90, 131.47, 130.65, 127.18, 126.35, 126.27, 119.60, 119.38, 111.62, 60.37, 55.93, 32.28, 31.97, 30.58 (d), 29.79 (d), 25.09, 24.98, 24.32 (t), 23.58, 23.45 ppm.

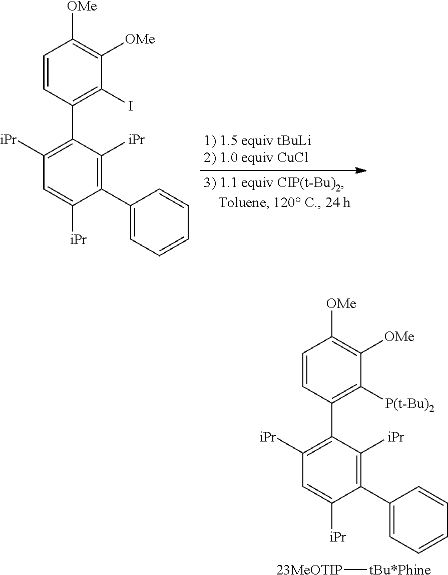

### Di-tert-butyl(2′,4′,6′-triisopropyl-3,4-dimethoxy-[1,1′:3′,1″-terphenyl]-2-yl)phosphane (23MeOTIP-tBu*Phine)

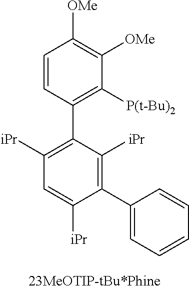

A 10 mL oven-dried Schlenk tube was charged with 23MeOTIP-iodide (271 mg, 0.5 mmol) and anhydrous toluene (1.0 mL). The tube was capped with a rubber septum and was cooled to −78° C. and 1.7 M n-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-butylchlorophosphine (108 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 24 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (183 mg, 65%), labeled as 23MeOTIP-tBu*Phine. ¹H NMR (600 MHz, Chloroform-d) δ 7.38-7.28 (m, 4H), 7.26-7.20 (m, 1H), 7.11 (s, 1H), 6.97-6.86 (m, 2H), 4.03 (s, 3H), 3.91 (s, 3H), 2.86-2.74 (m, 1H), 2.47 (dq, J=13.5, 6.6 Hz, 2H), 1.25 (d, J=6.9 Hz, 3H), 1.17 (dd, J=12.0, 5.4 Hz, 18H), 1.08 (dd, J=18.0, 6.9 Hz, 6H), 1.01 (d, J=6.6 Hz, 3H), 0.91 (d, J=7.0 Hz, 3H), 0.47 (d, J=7.2 Hz, 3H) ppm. ¹³C NMR (151 MHz, Benzene-d₆) δ 152.21, 149.94, 146.35, 145.49 (d, J_{CP}=13.5 Hz), 142.63, 142.01, 136.70, 132.62, 130.54, 127.88, 127.72, 127.56, 126.83, 126.70, 126.06, 118.52, 114.60, 112.96, 59.97, 55.72, 32.46, 31.42, 31.08, 29.28, 27.98, 26.56, 24.64, 24.36, 23.37, 22.65 ppm. ³¹P NMR (243 MHz, CDCl₃) δ=39.11 ppm.

### Synthesis of Ligand 23MeTIP-Ad*Phine

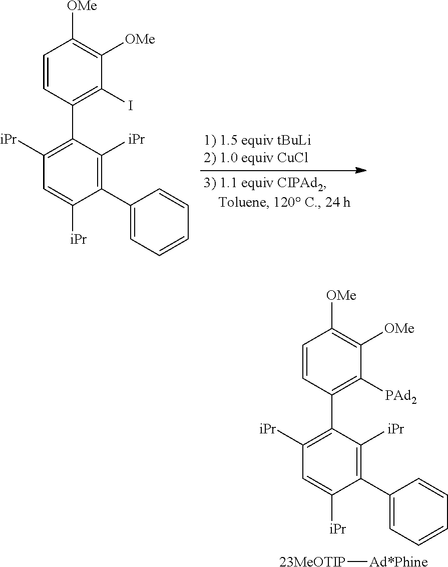

### Di((1S,3R,5R,7S)-adamantan-1-yl)(2′,4′,6′-triisopropyl-3,4-dimethoxy-[1,1′:3′,1″-terphenyl]-2-yl)phosphane (23MeOTIP-Ad*Phine)

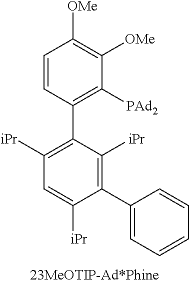

A 10 mL oven-dried Schlenk tube was charged with 23MeOTIP-I (263 mg, 0.5 mmol) and anhydrous toluene (1.0 mL). The tube was capped with a rubber septum and was cooled to −78° C. and 1.7 M n-BuLi solution in pentane (0.45 mL, 0.75 mmol, 1.5 equiv.) was added dropwise over 10 min under vigorous stirring. The mixture was stirred at −78° C. for 1 h (A). In another 10 mL oven-dried Schlenk tube was charged with CuCl (49.5 mg, 0.5 mmol, 1.0 equiv.). The tube was capped under nitrogen atmosphere and was cooled to −78° C. (B). A was transferred into to B by syringe over 15 min at −78° C. An additional 1 mL of anhydrous toluene was used to rinse the reaction and was also transferred to B. Subsequently, di-tert-adamantylchlorophosphine (202 mg, 0.6 mmol, 1.1 equiv.) in anhydrous toluene (1 mL) was injected slowly through syringe into the above mixture at −78° C. The combined reaction mixture was placed into preheated 120° C. oil bath and stirred vigorously for 48 h. At this time, the mixture was cooled room temperature, diluted with water (10 mL) and EtOAc (50 mL), and the layers were separated. The organic phase was washed with 28% NH₄OH (aq.) (20 mL×5), and brine (10 mL), successively. The combined aqueous phases were extracted with additional EtOAc (30 mL). The combined organic phases were dried over anhydrous MgSO₄ and filtered. After concentration, the crude product was recrystallized with MeOH to get a white solid (190 mg, 53%). ¹H NMR (600 MHz, Chloroform-d) δ 7.43-7.30 (m, 4H), 7.26-7.23 (m, 1H), 7.09 (s, 1H), 6.93 (d, J=8.3 Hz, 1H), 6.90 (d, J=3.9 Hz, 1H), 4.07 (s, 3H), 3.92 (s, 3H), 2.84-2.76 (m, 1H), 2.48 (dt, J=20.6, 6.8 Hz, 1H), 2.25-1.42 (m, 15H), 1.26 (d, J=6.8 Hz, 3H), 1.10 (d, J=6.8 Hz, 6H), 1.06 (d, J=6.9 Hz, 3H), 1.01 (d, J=6.7 Hz, 3H), 0.91 (d, J=7.0 Hz, 3H), 0.48 (d, J=7.2 Hz, 3H) ppm. ¹³C NMR (151 MHz, Benzene-d₆) δ 152.19 (d, J_{CP}=3.0 Hz), 149.66, 146.37 (d, J_{CP}=4.1 Hz), 143.82, 143.57, 142.96, 142.64, 142.18, 138.54, 136.62, 132.18, 130.58, 126.80, 126.68, 125.99, 119.23, 118.55, 113.01, 60.00, 55.71, 41.65 (dd, J_{CP}=21.4, 13.7 Hz), 40.71 (d, J_{CP}=11.1 Hz), 39.85 (d, J_{CP}=12.3 Hz), 38.87 (d, J_{CP}=13.5 Hz), 38.54, 38.36 (d, J_{CP}=4.2 Hz), 38.18, 38.05, 36.96 (d, J_{CP}=6.6 Hz), 36.74 (d, J_{CP}=7.6 Hz), 36.63-36.42 (m), 36.30 (d, J_{CP}=10.4 Hz), 35.36, 32.41 (d, J_{CP}=2.9 Hz), 31.08 (d, J_{CP}=2.3 Hz), 29.25, 28.73 (d, J_{CP}=7.7 Hz), 28.13 (d, J_{CP}=10.4 Hz), 26.61, 24.80, 24.38, 24.17 (d, J_{CP}=5.3 Hz), 23.47, 22.74 ppm. ³¹P NMR (243 MHz, CDCl₃) S=40.94 ppm.

### Synthesis of Ligand Npip-TIP-tBu*Phine

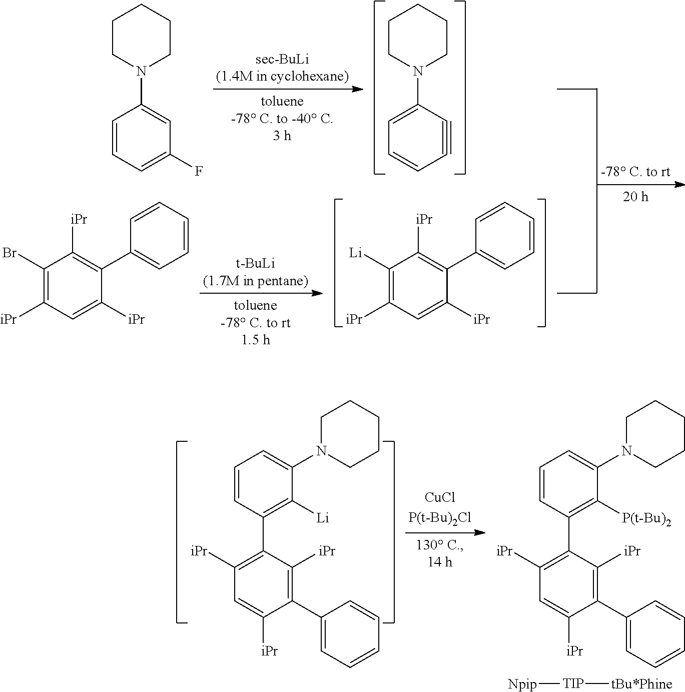

### 1-(2-(di-tert-butylphosphanyl)-2′,4′,6′-triisopropyl-[1,1:3′,1″-terphenyl]-3-yl)piperidine (Npip-TIP-tBu*Phine)

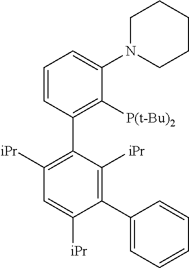

An oven-dried 20 mL microwave reaction vial (vial 1) which was equipped with a magnetic stir bar was charged with 1-(3-fluorophenyl)piperidine (0.28 g, 1.56 mmol) and anhydrous toluene (1.2 mL) inside a glove-box. The vial was sealed and brought out of the glove-box. The reaction mixture was cooled to −78° C. and sec-BuLi (1.4 M in cyclohexane, 1.23 mL, 1.72 mmol) was added dropwise in 10 min. The reaction mixture was allowed to warm to −40° C. in 2.5 h-3 h after which it was again cooled to −78° C. To a separate oven-dried 10 mL microwave reaction vial (vial 2) which was equipped with a magnetic stir bar was charged with 3-bromo-2,4,6-triisopropyl-1,1′-biphenyl (0.45 g, 1.25 mmol) and anhydrous toluene (1.8 mL) inside a glove-box. The vial was sealed and brought out of the glove-box. The reaction mixture was cooled to −78° C. and t-BuLi (1.7 M in pentane, 1.54 mL, 2.63 mmol) was added dropwise in 10 min. The reaction mixture was allowed to stir at −78° C. for 30 min after which the acetone:dry-ice bath was removed and the reaction mixture was allowed to stir at room temperature for 1 h. The above reaction mixture was added dropwise via a cannula to vial 1 at −78° C. in 20 min and the reaction mixture was allowed to warm to room temperature overnight (20 h). The above was again transferred to a glove-box and CuCl (155 mg, 1.56 mmol) was added to it. The above was stirred for 10 min before adding di-tert-butyl chlorophosphine (0.3 mL, 1.56 mmol) slowly. The vial was sealed, brought out of the glove-box and the reaction mixture was allowed to stir at 130° C. for 14 h. The reaction mixture was cooled and EtOAc (50 mL) and 30% NH₄OH (50 mL) were added. The two layers were shaken in a separatory funnel and separated. The organic phase was washed with 30% NH₄OH until the aqueous phase was no longer blue. The combined aqueous washes were back-extracted with EtOAc (100 mL) and the combined organic extracts were washed with brine, dried over Na₂SO₄, filtered and the solvent was removed with the aid of a rotary evaporator. The crude material was purified by trituration in 2:3 Et₂O:MeOH (2.0 mL) to afford an off-white colored solid (0.255 g, 35% yield), labeled as Npip-TIP-tBu*Phine. ¹H NMR (600 MHz, CDCl₃) δ 7.42-7.40 (m, 1H), 7.31-7.25 (m, 5H), 7.19-7.17 (m, 1H), 7.07-7.05 (m, 2H), 3.06 (bs, 2H), 2.74-2.64 (m, 3H), 2.45-2.40 (m, 1H), 2.25-2.20 (m, 1H), 1.77-1.62 (m, 6H), 1.20-1.66 (m, 20H), 1.06-1.03 (m, 7H), 0.93-0.92 (m, 3H), 0.86-0.84 (m, 3H), 0.45-0.43 (m, 3H); ¹³C NMR (150 MHz, CDCl₃) δ 161.22, 146.76, 146.30, 142.50, 142.41, 136.81, 132.41, 130.83, 130.17, 130.11, 128.24, 127.17, 127.00, 126.33, 122.59, 118.70, 34.11, 34.02, 33.90, 33.81, 33.18, 33.15, 32.67, 32.56, 32.44, 31.38, 29.56, 26.66, 25.86, 24.87, 24.67, 24.48, 24.30, 23.66, 22.86; ³¹P NMR (161 MHz, CDCl₃) δ 45.49.

### Synthesis of Ligand Npip-TIP-Ad*Phine

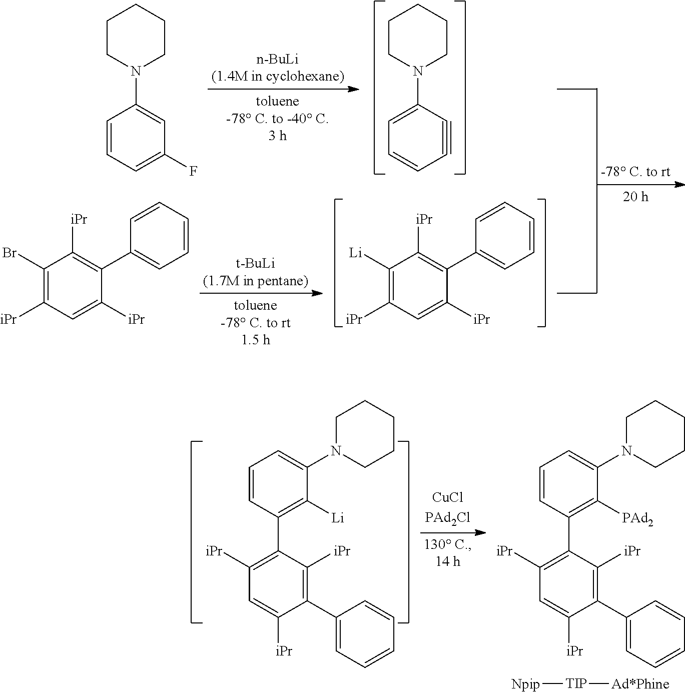

### 1-(2-(di((1s,3R,5R,7S)-adamantan-1-yl)phosphanyl)-2′,4′,6′-triisopropyl-[1,1′:3′,1″-terphenyl]-3-yl)piperidine (Npip-TIP-Ad*Phine)

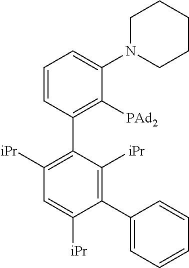

An oven-dried 20 mL microwave reaction vial (vial 1) which was equipped with a magnetic stir bar was charged with 1-(3-fluorophenyl)piperidine (0.30 g, 1.67 mmol) and anhydrous toluene (1.2 mL) inside a glove-box. The vial was sealed and brought out of the glove-box. The reaction mixture was cooled to −78° C. and n-BuLi (1.6 M in hexane, 1.15 mL, 1.84 mmol) was added dropwise in 10 min. The reaction mixture was allowed to warm to −40° C. in 2.5 h-3 h after which it was again cooled to −78° C. To a separate oven-dried 10 mL microwave reaction vial (vial 2) which was equipped with a magnetic stir bar was charged with 3-bromo-2,4,6-triisopropyl-1,1′-biphenyl (0.421 g, 1.17 mmol) and anhydrous toluene (1.8 mL) inside a glove-box. The vial was sealed and brought out of the glove-box. The reaction mixture was cooled to −78° C. and t-BuLi (1.7 M in pentane, 1.41 mL, 2.40 mmol) was added dropwise in 10 min. The reaction mixture was allowed to stir at −78° C. for 30 min after which the acetone:dry-ice bath was removed and the reaction mixture was allowed to stir at room temperature for 1 h. The above reaction mixture was added dropwise via a cannula to vial 1 at −78° C. in 20 min and the reaction mixture was allowed to warm to room temperature overnight (20 h). The above was again transferred to a glove-box and CuCl (165 mg, 1.67 mmol) was added to it. The above was stirred for 10 min before adding di(1-adamantyl)chlorophosphine (0.563 mg, 1.67 mmol). The vial was sealed, brought out of the glove-box and the reaction mixture was allowed to stir at 130° C. for 18 h. The reaction mixture was cooled and EtOAc (50 mL) and 30% NH₄OH (50 mL) were added. The two layers were shaken in a separatory funnel and separated. The organic phase was washed with 30% NH₄OH until the aqueous phase was no longer blue. The combined aqueous washes were back-extracted with EtOAc (100 mL) and the combined organic extracts were washed with brine, dried over Na₂SO₄, filtered and the solvent was removed with the aid of a rotary evaporator. The resulting mixture was suspended in Et₂O (3.0 mL) and allowed to stir at 0° C. for 1 h and filtered. The resulting filtrate was concentrated completely and the crude material was purified by trituration in 1:4 Et₂O: MeOH (3.0 mL) to afford an off-white solid (0.173 g, 20% yield), labeled as Npip-TIP-Ad*Phine. H NMR (600 MHz, CDCl₃) δ 7.41-7.39 (m, 1H), 7.33-7.24 (m, 5H), 7.20-7.18 (m, 1H), 7.09-7.07 (m, 1H), 7.05 (s, 1H), 3.24-3.19 (m, 2H), 2.78-2.63 (m, 3H), 2.45-2.40 (m, 1H), 2.23-2.19 (m, 1H), 2.15-2.02 (m, 2H), 2.00-1.91 (m, 4H), 1.84-1.80 (m, 12H), 1.76-1.70 (m, 6H), 1.61-1.58 (m, 12H), 1.20-1.18 (m, 3H), 1.05-1.02 (m, 6H), 0.94-0.92 (m, 3H), 0.88-0.86 (m, 3H), 0.48-0.46 (m, 3H); ¹³C NMR (150 MHz, CDCl₃) δ 161.38, 161.35, 154.82, 154.53, 146.76, 146.44, 142.57, 142.54, 140.04, 139.98, 136.68, 133.50, 132.44, 130.79, 130.09, 130.02, 128.01, 127.22, 127.00, 126.31, 122.11, 118.55, 59.61, 58.12, 42.55, 42.46, 42.15, 42.05, 39.30, 39.25, 3920, 39.12, 39.03, 38.91, 38.82, 37.42, 37.26, 33.28, 33.25, 31.28, 33.25, 31.41, 29.55, 29.37, 29.31, 28.65, 28.63, 26.84, 25.82, 25.72, 25.01, 24.56, 24.19, 23.77, 22.78; ³¹P NMR (161 MHz, CDCl₃) δ 46.35 ppm.

### Synthesis of Ligand Npip-TIP-Cy*Phine

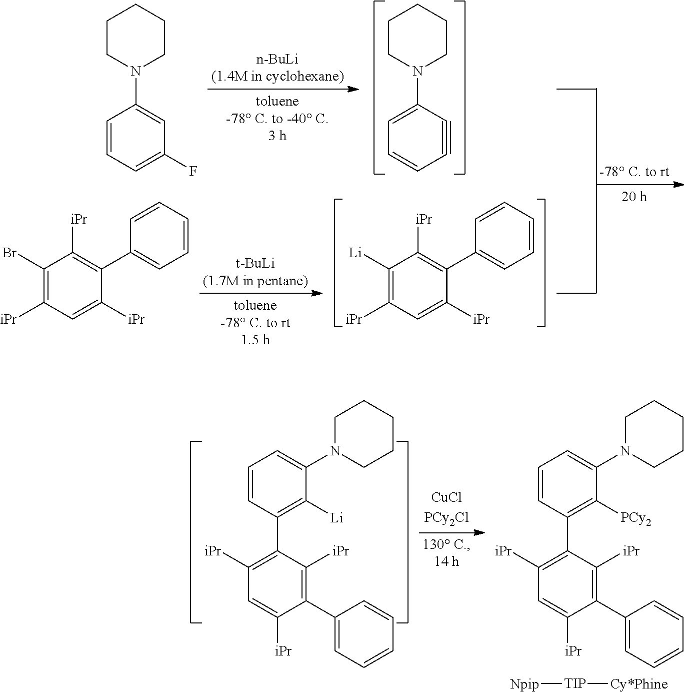

### 1-(2-(dicyclohexylphosphanyl)-2′,4′,6′-triisopropyl-[1,1′:3′,1″-terphenyl]-3-yl)piperidine (Npip-TIP-Cy*Phine)

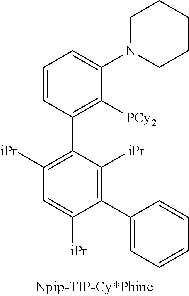

An oven-dried 20 mL microwave reaction vial (vial 1) which was equipped with a magnetic stir bar was charged with 1-(3-fluorophenyl)piperidine (0.30 g, 1.67 mmol) and anhydrous toluene (1.2 mL) inside a glove-box. The vial was sealed and brought out of the glove-box. The reaction mixture was cooled to −78° C. and n-BuLi (1.6 M in hexane, 1.15 mL, 1.84 mmol) was added dropwise in 10 min. The reaction mixture was allowed to warm to −40° C. in 2.5 h-3 h after which it was again cooled to −78° C. To a separate oven-dried 10 mL microwave reaction vial (vial 2) which was equipped with a magnetic stir bar was charged with 3-bromo-2,4,6-triisopropyl-1,1′-biphenyl (0.421 g, 1.17 mmol) and anhydrous toluene (1.8 mL) inside a glove-box. The vial was sealed and brought out of the glove-box. The reaction mixture was cooled to −78° C. and t-BuLi (1.7 M in pentane, 1.41 mL, 2.40 mmol) was added dropwise in 10 min. The reaction mixture was allowed to stir at −78° C. for 30 min after which the acetone:dry-ice bath was removed and the reaction mixture was allowed to stir at room temperature for 1 h. The above reaction mixture was added dropwise via a cannula to vial 1 at −78° C. in 20 min and the reaction mixture was allowed to warm to room temperature overnight (20 h). The above was again transferred to a glove-box and CuCl (165 mg, 1.67 mmol) was added to it. The above was stirred for 10 min before adding chlorodicyclohexylphosphine (0.37 mL, 1.67 mmol). The vial was sealed, brought out of the glove-box and the reaction mixture was allowed to stir at 130° C. for 8 h. The reaction mixture was cooled and EtOAc (50 mL) and 30% NH₄OH (50 mL) were added. The two layers were shaken in a separatory funnel and separated. The organic phase was washed with 30% NH₄OH until the aqueous phase was no longer blue. The combined aqueous washes were back-extracted with EtOAc (100 mL) and the combined organic extracts were washed with brine, dried over Na₂SO₄, filtered and the solvent was removed with the aid of a rotary evaporator. The crude material was purified by trituration in 2:3 acetone:MeOH (2.0 mL) twice to afford an off-white solid (0.223 mg, 30% yield), labeled as Npip-TIP-Cy*Phine. ¹H NMR (600 MHz, C₆D₆) δ 7.48 (s, 1H), 7.45-7.41 (m, 2H), 7.22-7.18 (m, 4H), 7.15-7.12 (m, 2H), 3:19-3.12 (m, 1H), 2.92-2.84 (m, 2H), 2.78-2.66 (m, 4H), 2.54-2.48 (m, 2H), 2.09-2.01 (m, 2H), 1.83-1.58 (m, 12H), 1.52-1.51 (m, 3H), 1.38-1.22 (m, 14H), 1.18-1.12 (m, 10H), 0.98-0.97 (m, 3H); ¹³C NMR (150 MHz, CDCl₃) δ 160.79, 147.72, 142.89, 138.36, 132.94, 131.60, 128.81, 128.68, 127.82, 127.76, 127.15, 119.88, 33.35, 31.61, 30.24, 28.20, 28.14, 28.06, 27.40, 27.27, 27.15, 27.00, 25.26, 25.17, 25.08, 24.74, 24.49, 23.77; ³¹P NMR (161 MHz, CDCl₃) δ 1.23 ppm.

### Palladium-*Phine Complexes

### Synthesis of PdCl₂(TIP-Cy*Phine)₂

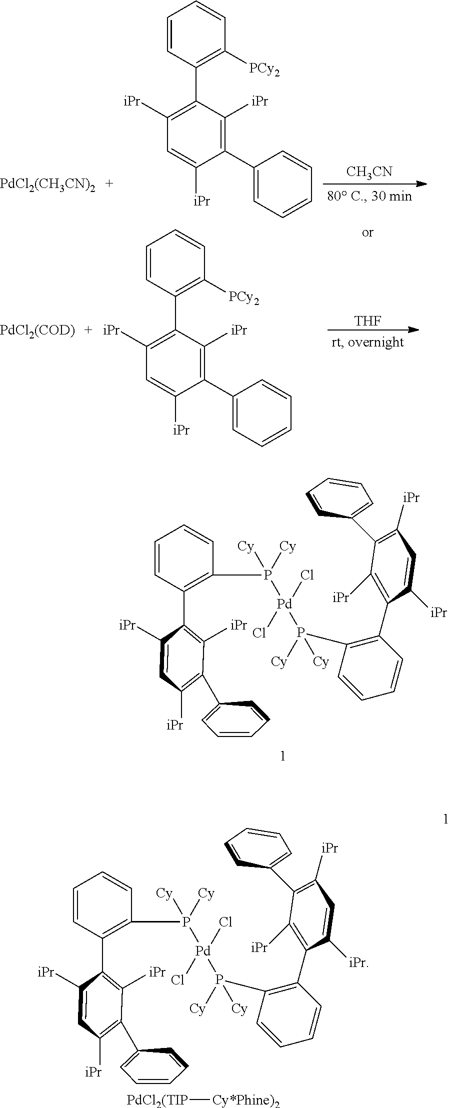

To an oven-dried vial was added PdCl₂(CH₃CN)₂ (520 mg, 2 mmol, 1.0 equiv.) and anhydrous acetonitrile (20 mL). With rapid stirring, TIP-Cy*Phine (L, 1.1 g, 4 mmol, 2.0 equiv.) was added potion-by-potion. The vial was capped under nitrogen and placed into an 80° C. preheated oil bath for 30 min with vigorously stirring, during which period an orange precipitate formed. The precipitate was filtered through a sintered glass frit, washed with pentane (3×8 mL), and dried under reduced pressure to afford a yellow solid (1.7 g, 70%). Alternative procedure: To an oven-dried vial was added PdCl₂(COD) (28.6 mg, 0.1 mmol, 1.0 equiv.) and anhydrous THF (5 mL). With rapid stirring, TIP-Cy*Phine (L, 110.8 mg, 0.2 mmol, 2.0 equiv.) was added. The vial was capped under nitrogen and stirred vigorously at room temperature overnight. The solvent was removed in vacuo and pentane (10 mL) was added. The yellow solid precipitates out immediately and was filtered through a sintered glass frit, washed with pentane (3×5 mL), and dried under reduced pressure to afford a yellow solid (111 mg, 85%). ³¹P NMR (243 MHz, CDCl₃) δ=45.3.

### Synthesis of PdBr(TIP-tBu*Phine)

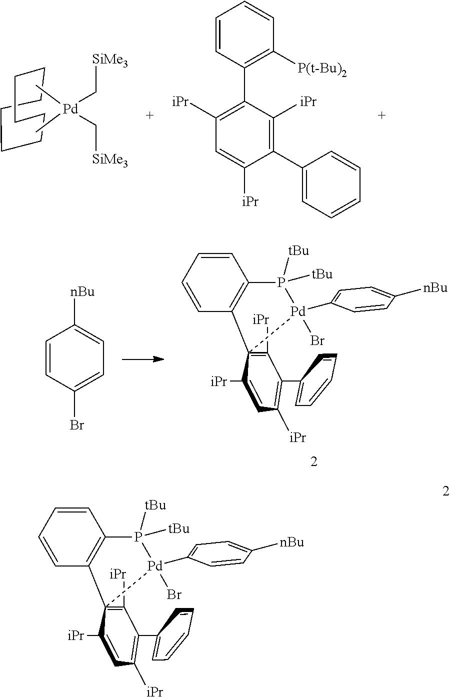

To an oven-dried vial was added TIP-tBu*Phine (450 mg, 0.9 mmol, 1.0 equiv.) and 4-bromo-nbutylbenzene (959 mg, 4.5 mmol, 5 equiv.). With rapid stirring, cyclohexane was added dropwise until all reagents had completely dissolved (3 mL total). (COD)Pd(CH2TMS)2 (350 mg, 0.9 mmol, 1.0 equiv.) was added rapidly in one portion and the mixture was vigorously stirred at room temperature for 16 hours, during which period a precipitate formed. Pentane (2 ml) was added and the mixture placed in a −30° C. freezer for at least 1 hour. The mixture was filtered through a sintered glass frit, washed with pentane (3×6 mL), and dried under reduced pressure to afford 2 as a bright yellow solid (570 mg, 77%). ¹H NMR (400 MHz, Methylene Chloride-d₂) δ 8.05 (ddd, J=7.8, 6.0, 1.8 Hz, 1H), 8.01-7.89 (m, 1H), 7.54-7.36 (m, 5H), 7.34-7.30 (m, 1H), 7.06 (dd, J=8.4, 1.9 Hz, 2H), 6.97-6.88 (m, 1H), 6.76 (d, J=8.0 Hz, 3H), 5.56-4.94 (m, 2H), 3.85 (s, 2H), 2.91 (p, J=7.1 Hz, 1H), 2.54 (td, J=8.9, 8.2, 6.5 Hz, 4H), 1.64 (d, J=6.7 Hz, 3H), 1.50 (d, J=14.0 Hz, 10H), 1.37 (d, J=14.0 Hz, 10H), 1.33 (s, 3H), 1.09 (d, J=6.8 Hz, 3H), 1.04-0.89 (m, 10H), 0.86 (d, J=7.0 Hz, 3H). ppm. ¹³C NMR (151 MHz, Chloroform-d) δ 156.99, 151.47, 148.70, 148.25 (d, J_{CP}=19.5 Hz), 140.69, 139.59, 138.62, 137.98, 136.95, 136.39 (d, J_{CP}=23.4 Hz), 135.30, 135.14 (d, J_{CP}=10.5 Hz), 132.81, 130.98, 129.63, 127.11, 126.76 (d, J_{CP}=15.8 Hz), 126.60, 126.27, 125.37 (d, J_{CP}=4.1 Hz), 123.43, 39.61 (d, J_{CP}=14.4 Hz), 39.34 (d, J_{CP}=14.3 Hz), 34.60, 33.71, 33.21, 31.93 (d, J_{CP}=5.0 Hz), 31.50 (d, J_{CP}=4.9 Hz), 30.02, 25.54 (d, J_{CP}=12.1 Hz), 25.03, 24.72, 24.55, 23.25, 22.24, 14.00 ppm. ³¹P NMR (162 MHz, Methylene Chloride-d₂) δ 49.88 ppm.

### Synthesis of PdBr(TIP-tBu*Phine)

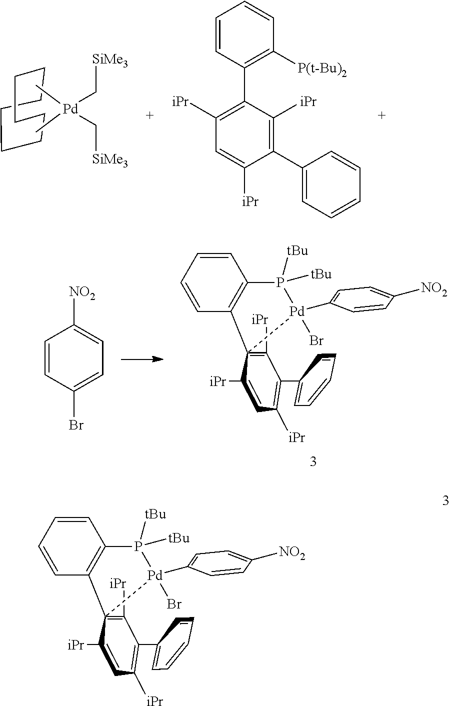

To an oven-dried vial was added TIP-tBu*Phine (250 mg, 0.5 mmol, 1.0 equiv.) and 4-bromo-nitrobenzene (502 mg, 0.5 mmol, 5 equiv.). With rapid stirring, cyclohexane was added dropwise until all reagents had completely dissolved (2 mL total). (COD)Pd(CH2TMS)2 (209 mg, 0.5 mmol, 1.0 equiv.) was added rapidly in one portion and the mixture was vigorously stirred at room temperature for 16 hours, during which period a precipitate formed. Pentane (3 ml) was added and the mixture placed in a −30° C. freezer for at least 1 hour. The mixture was filtered through a sintered glass frit, washed with pentane (3×6 mL), and dried under reduced pressure to afford 3 as a bright yellow solid (291 mg, 71%). ¹H NMR (600 MHz, Benzene-d₆) δ 7.85-7.75 (m, 2H), 7.62 (td, J=6.5, 5.9, 3.4 Hz, 2H), 7.56 (s, 1H), 7.54-7.48 (m, 1H), 7.41-7.32 (m, 2H), 7.01 (tt, J=7.6, 1.7 Hz, 1H), 6.96 (t, J=7.6 Hz, 1H), 6.70 (ddd, J=7.8, 3.5, 1.5 Hz, 1H), 3.04 (p, J=7.0 Hz, 1H), 2.97 (p, J=6.8 Hz, 1H), 2.61 (p, J=6.7 Hz, 1H), 1.79 (d, J=6.8 Hz, 3H), 1.64 (d, J=6.8 Hz, 3H), 1.36 (d, J=7.2 Hz, 3H), 1.26 (d, J=6.8 Hz, 12H), 1.09 (d, J=14.2 Hz, 12H), 0.99 (d, J=6.8 Hz, 3H) ppm. ³¹P NMR (162 MHz, Benzene-d₆) δ 50.44 ppm.

### Synthesis of PdBr(TIP-tBu*Phine)

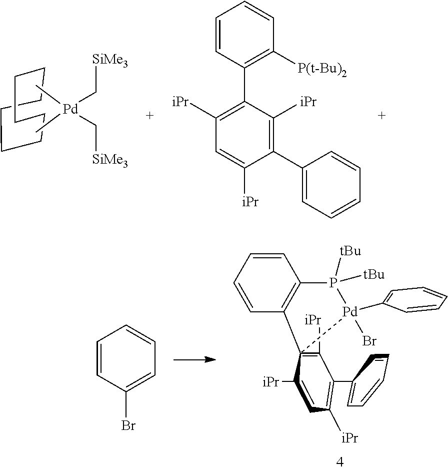

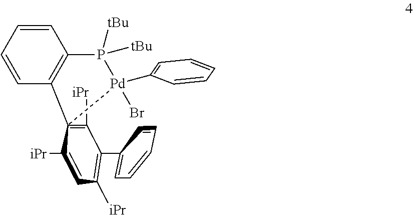

To an oven-dried vial was added TIP-tBu*Phine (36 mg, 0.07 mmol, 1.0 equiv.) and benzenebromide (56.2 mg, 0.07 mmol, 5 equiv.). With rapid stirring, cyclohexane was added dropwise until all reagents had completely dissolved (1 mL total). (COD)Pd(CH2TMS)2 (30 mg, 0.07 mmol, 1.0 equiv.) was added rapidly in one portion and the mixture was vigorously stirred at room temperature for 16 hours, during which period a precipitate formed. Pentane (2 ml) was added and the mixture placed in a −30° C. freezer for at least 1 hour. The mixture was filtered through a sintered glass frit, washed with pentane (3×6 mL), and dried under reduced pressure to afford 4 as a bright yellow solid (31 mg, 5%). ¹H NMR (400 MHz, THF-d₈) δ 8.12 (ddd, J=8.0, 6.1, 1.8 Hz, 1H), 8.06-7.99 (m, 1H), 7.50-7.41 (m, 2H), 7.34 (dtt, J=5.8, 4.0, 2.3 Hz, 3H), 7.13 (dd, J=6.8, 1.4 Hz, 2H), 6.94 (ddd, J=7.0, 3.4, 1.9 Hz, 1H), 6.76 (dd, J=8.2, 6.9 Hz, 2H), 6.71-6.62 (m, 1H), 2.94 (p, J=7.1 Hz, 1H), 2.57 (dp, J=13.7, 6.8 Hz, 2H), 1.63 (d, J=6.8 Hz, 3H), 1.50 (d, J=13.8 Hz, 9H), 1.37 (d, J=13.8 Hz, 9H), 1.32 (d, J=6.9 Hz, 3H), 1.06 (d, J=6.8 Hz, 3H), 1.00 (d, J=7.2 Hz, 3H), 0.93 (d, J=6.7 Hz, 3H), 0.85 (d, J=7.0 Hz, 3H) ppm. ¹³C NMR (151 MHz, THF-ds) δ 154.65, 149.40, 146.49, 146.16 (d, J_{CP}=19.7 Hz), 139.02, 137.67, 137.09, 134.46 (d, J_{CP}=22.8 Hz), 133.84, 133.37 (d, J_{CP}=10.5 Hz), 130.71, 129.34 (d, J_{CP}=2.4 Hz), 129.14, 127.85 (d, J_{CP}=2.2 Hz), 125.57 (d, J_{CP}=3.2 Hz), 124.95, 124.72, 124.34, 123.87-123.06 (m), 121.32, 120.25, 37.70-37.17 (m), 37.09, 31.26, 29.47, 29.07 (d, J_{CP}=4.9 Hz), 28.00, 23.18-22.34 (m), 20.79 ppm. ³¹P NMR (162 MHz, THF-ds) δ 49.13 ppm.

### *Phinite Ligand Series

### Overall Scheme for Synthesis of *Phinite Ligands and Pd*Phinite Precatalysts

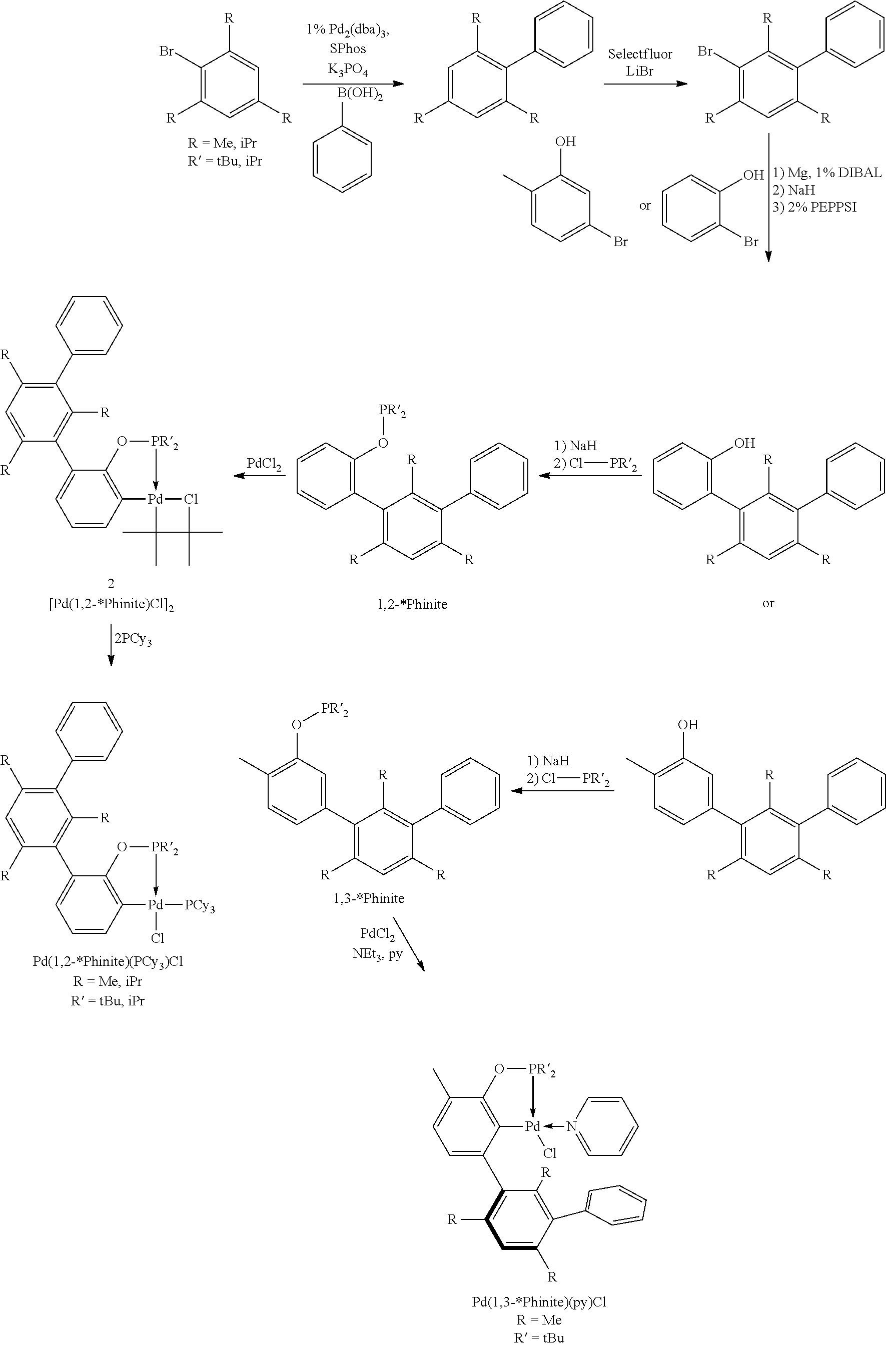

### Synthesis 1,2-tBu*Phinite

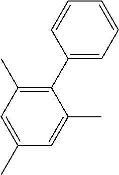

2,4,6-Trimethyl-1,1′-biphenyl. To an oven dried 2 neck 50 mL flask equipped with a magnetic stir bar and condenser, 1.98 g (10 mmol) of mestlyene bromide, 2.44 g (15 mmol) of phenylbornic acid, 4.224 g (20 mmol) of potassium phosphate tribasic, 0.820 g (2 mol %) of SPhos and 0.910 g (1 mol %) of tris(dibenzylideneacetone)dipalladium(0) was added. The mixture was then evacuated and backfilled with argon three times then anhydrous tetrahydrofuran (10 mL) and toluene (10 mL) were added to the flask. The mixture was stirred at 60° C. for 16 h then cooled to room temperature prior to filtration through a pad of Celite, washing thoroughly with EtOAc. The red filtrate was concentrated in vacuo yielding a crude residue that was purified by flash chromatography on silica gel using hexanes as the eluent to afford a colorless liquid (1.56 g, 80%). ¹H (600 MHz, CDCl₃) δ: 7.59 (t, J=7.44 Hz, 2H), 7.51-7.49 (m, 1H), 7.33 (dd, J=6.84 Hz, 1.38 Hz, 2H), 7.14 (s, 2H), 2.53 (s, 3H), 2.21 (s, 6H). ¹³C (150 MHz, CDCl₃) δ: 141.34, 139.28, 136.69, 136.11, 129.49, 128.58, 128.28, 126.71, 21.24, 20.95.

### 3-Bromo-2,4,6-trimethyl-1,1′-biphenyl

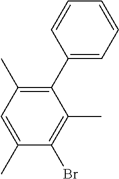 [SMILES: Cc1cc(C)c(-c2ccccc2)c(C)c1Br]

To a 250 mL flask wrapped with aluminium foil equipped with a magnetic bar was charged with 1.96 g (10 mmol) of 2,4,6-trimethyl-1,1′-biphenyl, 0.869 g (10 mmol) of lithium bromide, 3.98 g (11 mmol) of selectfluor and acetonitrile (70 mL). The mixture was stirred at room temperature for 16 h after which the solvent was removed under vacuum. The residue was extracted into dichloromethane and filtered through a pad of Celite. The filtrate was collected and washed with concentrated sodium thiosulfate. The organic layer was collected, dried with MgSO₄, filtered and concentrated to dryness under reduced pressure. The crude residue was then purified by flash chromatography on silica gel using hexanes and the eluent to afford a colorless liquid (0.220 g, 80%). ¹H (600 MHz, CDCl₃) δ: 7.42 (t, J=7.3 Hz, 2H), 7.35-7.33 (m, 1H), 7.09 (dd, J=6.84, 1.44 Hz, 2H), 7.02 (s, 1H), 2.43 (s, 3H), 2.12 (s, 3H), 1.93 (s, 3H). ¹³C (150 MHz, CDCl₃) δ: 141.45, 141.11, 137.13, 136.25, 135.01, 129.62, 129.32, 128.73, 127.07, 125.60, 24.20, 22.19, 20.81.

### 2′,4′,6′-trimethyl-[1,1′:3′,1″-terphenyl]-2-ol

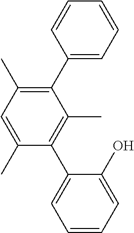 [SMILES: Cc1cc(C)c(-c2ccccc2O)c(C)c1-c1ccccc1]

To an oven-dried flask fitted with a magnetic stir-bar was sequentially added 194.5 mg of Mg (8.0 mmol), 2 mL of THF and 80 μL (0.08 mmol) of a 1.0 M solution of DIBAL in THF.

After stirring the mixture for 2 min at 25° C., 1.65 g (6.0 mmol) of 3-Bromo-2,4,6-trimethyl-1,1′-biphenyl was added and stirred overnight (16 h) at 25° C. until completion as judged by GC-MS. In a separate flask, a magnetic stir-bar, 100.8 mg (4.2 mmol) of NaH, 1.0 mL of THF was added, followed by the dropwise addition of 692 mg (4.0 mmol) of 2-bromophenol at 25° C. The mixture was stirred for 10 min (until the effervescence ceases) at which point the white suspension has become a transparent solution and 54.4 mg (0.08 mmol) of PEPPSI-IPr is added. The Grignard solution formed earlier was then diluted with 1 mL of THF and rapidly added (in one shot) to the other mixture, which resulted in the formation of a mustard yellow suspension. The flask was quickly sealed under argon and heated in a 70° C. oil bath for 16 h. After the reaction was complete, as determined by GC-MS, the resultant grey suspension was cooled to room temperature and quenched with 6.0 mL of a 0.5 M aq Na₃EDTA solution. The crude product was extracted with 3×20 mL of EtOAc. The organic portions were combined, washed with H₂O and brine, dried with MgSO₄, filtered and concentrated in vacuo affording viscous yellow oil. The crude product was purified by flash chromatography on silica gel using petroleum ether/EtOAc (0 to 20% EtOAc) as the column eluent to yield a white solid. The product can be recrystallized using a 95:5 petroleum ether/EtOAc mixture to give colorless crystalline blocks (865 mg, 75%). ¹H NMR (600 MHz, Benzene-d₆) δ 7.23-7.18 (m, 2H), 7.14-7.01 (m, 5H), 6.98 (dd, J=7.5, 1.7 Hz, 1H), 6.94 (s, 1H), 6.85 (td, J=7.4, 1.3 Hz, 1H), 4.58 (s, 1H), 2.03 (s, 3H), 2.02 (s, 3H), 1.91 (s, 3H). ¹³C NMR (151 MHz, Benzene-d₆) δ 153.68, 142.12, 141.23, 137.14, 136.71, 136.21, 133.57, 130.73, 130.24, 129.87, 129.85, 129.68, 129.24, 129.22, 128.68, 127.31, 121.42, 116.14, 21.41, 20.74, 19.01.

### 2′,4′,6′-triisopropyl-[1,1′:3′,1″-terphenyl]-2-ol

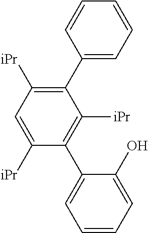 [SMILES: CC(C)c1cc(C(C)C)c(-c2ccccc2O)c(C(C)C)c1-c1ccccc1]

The procedure used was the same as 2′,4′,6′-trimethyl-[1,1′:3′,1″-terphenyl]-2-ol with the exception of the usage of 2.16 g (6 mmol) of 3-bromo-2,4,6-triisopropyl-1,1′-biphenyl as the starting material (969 mg, 65%). ¹H NMR (600 MHz, Chloroform-d) δ 7.36 (m, 2H), 7.34-7.30 (m, 1H), 7.28-7.25 (m, 1H), 7.25 (s, 1H), 7.23-7.20 (m, 1H), 7.19-7.16 (m, 1H), 7.10 (d, J=7.5 Hz, 1H), 6.97-6.92 (m, 2H), 4.72 (br, 1H), 2.83 (p, J=7.3 Hz, 1H), 2.58-2.42 (m, 2H), 1.15 (d, J=6.9 Hz, 3H), 1.08 (t, J=7.0 Hz, 6H), 1.04 (d, J=6.8 Hz, 3H), 0.80-0.75 (m, 3H), 0.73 (d, J=7.3 Hz, 3H). ¹³C NMR (151 MHz, Chloroform-d) δ 153.63, 148.67, 145.15, 141.48, 139.24, 129.12, 127.84, 127.32, 126.79, 120.66, 120.01, 114.82, 32.30, 30.32, 24.89, 24.40, 24.30, 24.13, 23.43.

### Di-tert-butyl((2′,4′,6′-trimethyl-[1,1′:3′,1″-terphenyl]-2-yl)oxy)phosphane (1,2-tBu*Phinite)

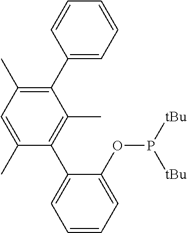 [SMILES: Cc1cc(C)c(-c2ccccc2OP(C(C)(C)C)C(C)(C)C)c(C)c1-c1ccccc1]

To an oven-dried flask fitted with a magnetic stir-bar was added 50 mg (2.2 mmol) of NaH and 5 mL of THF. To the stirring white suspension, 577 mg (2.0 mmol) of 2′,4′,6′-trimethyl-[1,1′:3′,1″-terphenyl]-2-ol dissolved in 10 mL of THF was added dropwise at 25° C. After effervescence ceases, 394 mg (2.18 mmol) of P(tBu)₂Cl was added dropwise, which resulted in the emergence of a fine suspension. The flask was sealed and heated in a 70° C. oil bath for 16 h. After cooling to room temperature, 10 mL of petroleum ether was added and the mixture was filtered through a pad of Celite. The colorless filtrate was concentrated to near dryness and the addition of 5 mL of anhydrous MeOH resulted in the precipitation of a white solid. The solid was collected by vacuum filtration and washed with pentane to afford a pure white powder (692 mg, 80%). ¹H NMR (600 MHz, Benzene-d₆) δ 7.86 (ddd, J=8.4, 4.4, 1.1 Hz, 1H), 7.46-7.42 (m, 1H), 7.41-7.38 (m, 1H), 7.37-7.33 (m, 1H), 7.29-7.25 (m, 3H), 7.25 (d, J=1.7 Hz, 1H), 7.22 (dd, J=7.4, 1.8 Hz, 1H), 7.01 (td, J=7.4, 1.1 Hz, 1H), 2.30 (s, 3H), 2.18 (s, 3H), 2.13 (s, 3H), 1.10 (d, J=0.6 Hz, 9H), 1.10 (d, J=22.7 Hz, 9H). ¹³C NMR (151 MHz, Benzene-d₆) δ 157.19 (d, J=9.3 Hz), 143.11, 140.47, 136.99, 135.82, 135.05, 134.86, 132.18, 131.61, 130.06, 129.89, 129.32 (d, J=6.6 Hz), 129.17, 128.90, 127.11, 121.89, 117.46, 117.31, 36.07 (d, J=3.4 Hz), 35.90 (d, J=3.3 Hz), 27.80 (d, J=3.5 Hz), 27.70 (d, J=3.7 Hz), 21.46, 21.39, 19.84. ³¹P NMR (162 MHz, Benzene-d₆) δ 146.70.

### Synthesis 1,2-iPr*Phinite

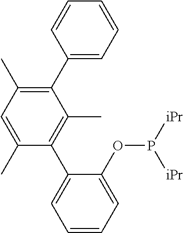 [SMILES: Cc1cc(C)c(-c2ccccc2OP(C(C)C)C(C)C)c(C)c1-c1ccccc1]

Diisopropyl((2′,4′,6′-trimethyl-[1,1′:3′,1″-terphenyl]-2-yl)oxy)phosphane (1,2-iPr-*Phinite). The procedure used was the same as 1,2-tBu*Phinite with the exception of the usage of 335 mg (2.2 mmol) of P(iPr)₂Cl as starting material (485 mg, 60%). ¹H NMR (400 MHz, Benzene-d₆) δ 7.58 (ddd, J=8.2, 3.5, 1.1 Hz, 1H), 7.36-7.20 (ni, 4H), 7.15-7.06 (m, 3H), 7.04 (s, 1H), 6.91 (td, J=7.4, 1.2 Hz, 1H), 2.17 (s, 3H), 2.08 (s, 3H), 2.00 (s, 3H), 1.65-1.57 (m, 2H), 0.99-0.83 (m, 12H). ¹³C NMR (151 MHz, Benzene-d₆) δ 156.65 (d, J=8.6 Hz), 143.05, 140.31, 137.03, 135.94, 135.03, 134.98, 131.58, 130.12, 129.91, 129.30, 129.27, 129.16, 128.87, 128.82, 127.10, 122.27, 118.02 (d, J=19.7 Hz), 28.77 (d, J=7.9 Hz), 28.65 (d, J=8.0 Hz), 21.42, 21.35, 19.58, 18.18, 18.04, ³¹P NMR (162 MHz, Benzene-d₆) δ 144.32.

### Synthesis 1,2-tBu*Phinite-TIP

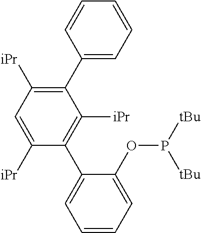 [SMILES: CC(C)c1cc(C(C)C)c(-c2ccccc2OP(C(C)(C)C)C(C)(C)C)c(C(C)C)c1-c1ccccc1]

Di-tert-butyl((2′,4′,6′-triisopropyl-[1,1′:3′,1″-terphenyl]-2-yl)oxy)phosphane (1,2-tBu*Phinite-TIP). The procedure used was the same as 1,2-tBu*Phinite with the exception of the usage of 745 mg (2 mmol) of 2′,4′,6′-triisopropyl-[1,1′:3′,1″-terphenyl]-2-ol as starting material (827 mg, 80%). ¹H NMR (600 MHz, Benzene-d₆) δ 7.85 (ddd, J=8.3, 5.4, 1.1 Hz, 1H), 7.43 (s, 1H), 7.40-7.32 (m, 3H), 7.26-7.17 (m, 3H), 7.15-7.11 (m, 1H), 6.91 (td, J=7.4, 1.1 Hz, 1H), 3.11 (hept, J=7.1 Hz, 1H), 2.78 (heptd, J=6.8, 4.3 Hz, 2H), 1.36 (d, J=6.9 Hz, 3H), 1.17 (d, J=6.9 Hz, 3H), 1.12 (d, J=6.7 Hz, 3H), 1.04 (d, J=5.9 Hz, 9H), 1.02 (d, J=5.9 Hz, 9H). ¹³C NMR (151 MHz, Benzene-d₆) δ 156.52 (d, J=9.5 Hz), 147.01, 146.52, 143.17, 142.02, 137.57, 134.75, 132.23, 131.57, 130.99, 130.61, 128.22 (d, J=2.5 Hz), 128.09, 127.06, 126.50, 120.13, 118.86, 116.24 (d, J=25.6 Hz), 35.22 (d, J=26.2 Hz), 35.10 (d, J=25.7 Hz), 32.38, 30.77, 29.63, 27.14 (dd, J=15.6, 10.0 Hz), 26.31, 24.42, 22.30. ³¹P NMR (243 MHz, Benzene-d₆) δ 143.70.

### Synthesis 1,3-tBu*Phinite

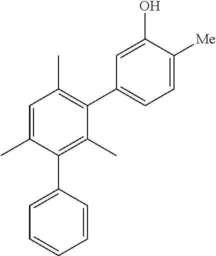 [SMILES: Cc1ccc(-c2c(C)cc(C)c(-c3ccccc3)c2C)cc1O]

2′,4,4′,6′-Tetramethyl-[1,1′:3′,1″-terphenyl]-3-ol. To a 5 mL oven dried microwave vial equipped with a magnetic stir bar 26.4 mg (1.1 mmol) of magnesium and 2 mol % of DiBal-H was added and allow to stir for 5 min in the glove box. 0.414 g (1.5 mmol) of 3-bromo-2,4,6-trimethyl-1,1′-biphenyl was dissolve in dry tetrahydrofuran (0.3 mL). The vial was seal and allowed to stir at 70° C. for 2 h. The mixture will the turn into a viscous brown liquid. To another 5 mL oven dried microwave vial equipped with a magnetic stir bar 25.2 mg (1.05 mmol) of sodium hydride was mix with dry tetrahydrofuran (0.5 mL) in the glove box was allowed to stir for 5 min next 0.187 g (1 mmol) of 5-bromo-2-methylphenol was added slowly into the mixture until the bubbling stop and the mixture turn into a clear solution then 14 mg (2 mol %) of PEPPSI-iPr was added. The Grignard was then diluted with dry tetrahydrofuran (0.5 mL) and added to the second microwave vial fast in the glove box. The vial was then seal in argon and allowed to stir for 18 h at 70° C. in a preheated oil bath outside of the glove box. The Reaction was diluted with Na₃EDTA 0.5 M, extract with diethyl ether 3 time the organic layer was then wash with water 2 times and brine 2 time. The organic layer was collected and dry with MgSO₄. The crude was purified by column (hexane: ethyl acetate 10:1) ratio to afford a white solid (211 mg, yield 70%). ¹H (600 MHz, CDCl₃) δ 7.47 (t, J=7.8 Hz, 2H), 7.38 (t, J=7.2 Hz, 1H), 7.24-7.22 (m, 3H), 7.10 (s, 1H), 6.73 (dd, J=6, 1.2 Hz, 1H), 6.65 (s, 1H), 4.90 (s, 1H), 2.35 (s, 3H), 2.12 (s, 3H), 2.09 (s, 3H), 1.78 (s, 3H). ¹³C (150 MHz, CDCl₃) δ: 153.75, 141.66, 140.72, 139.72, 139.34, 135.01, 134.90, 134.21, 131.13, 129.38, 129.36, 128.53, 128.47, 126.54, 121.86, 115.87, 20.88, 20.83, 19.03, 15.61.

### Di-tert-butyl((2′,4,4′,6′-tetramethyl-[1,1′:3′,1″-terphenyl]-3-yl)oxy)phosphane (1,3-tBu*Phinite)

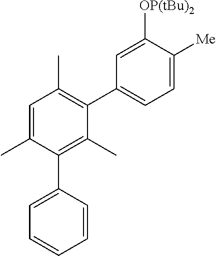

In an oven dried 5 mL microwave vial equipped with a magnetic stir bar 25.5 mg (1.05 mmol) of sodium hydride was mix with dry tetrahydrofuran (0.5 mL) and was allowed to stir of 5 min. 0.302 g (1 mmol) of 2′,4,4′,6′-tetramethyl-[1,1′:3′,1″-terphenyl]-3-ol was dissolve in dry tetrahydrofuran (0.5 mL) and added slowly to the vial until the bubbling stop and the mixture will then turned into a clear solution. 0.216 g (1.2 mmol) of di-tert-butylcholrophosohine was added drop-wise to the vial the mixture then turn into a white suspension again in the glove box. The vial was then seal in argon and allowed to stir at 70° C. for 16 h. Reaction was cool to room temperature, filter through celite and solvent was removed. The crude was then purified by re-crystallization with dry methanol in the glove box to afford a white solid (335 mg, yield 75%). ¹H (600 MHz, C₆D₆) δ: 7.62 (dd, ⁴J_{H,H}=3.24 1.5 Hz, 1H), 7.16-7.13 (m, 2H), 7.08-7.06 (m, 2H), 7.04 (d, ³J_{H,H}=7.5 Hz, 1H), 6.97 (d, ³J_{H,H}=1.08 Hz, 1H), 6.96 (s, 1H), 6.71 (dd, ³J_{H,H}=5.94 Hz, 1.34 Hz, 1H), 2.29 (s, 3H), 2.15 (s, 3H), 2.03 (s, 3H), 2.00 (s, 3H), 1.05 (d, ³J_{H,H}=10.4 Hz, 9H), 1.02 (d, ³J_{H,H}=11.4 Hz, 9H). ¹³C (150 MHz, C₆D₆) δ:157.86, 157.79, 142.10, 140.63, 1329.94, 139.89, 134.68, 134.37, 133.69, 131.07, 129.41, 129.27, 128.87, 128.42, 128.38, 128.31, 126.31, 125.06, 121.77, 117.41, 117.26, 67.44, 35.33, 35.16, 27.15, 27.13, 27.05, 27.03, 25.15, 20.75, 20.67, 19.02, 16.32. ³¹P {¹H} (161 MHz, C₆D₆) δ: 146.5.

### Pd*Phinite Precatalyst Series

### Synthesis [Pd(1,2-tBu*Phinite)Cl]₂

### [Pd(1,2-tBu*Phinite)Cl]₂

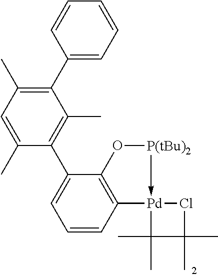

An oven-dried flask fitted with a magnetic stir-bar was charged with 910 mg (2.1 mmol) of 1,2-tBu*Phinite, 355 mg (2 mmol) of PdCl₂ and 50 mL of toluene. The flask was sealed, stirred and heated in a 110° C. oil bath for 16 h. After cooling to room temperature, the yellow solution was filtered through a pad of Celite and concentrated to dryness in vacuo to yield a yellow solid. The crude product was dissolved in 10 mL of CH₂Cl₂, filtered through Celite and concentrated to near dryness prior to the addition of 5 mL of anhydrous MeOH to induce precipitation. The pale yellow powder was collected by vacuum filtration and washed with pentane to afford the pure product (1.38 g, 60%). ¹H NMR (600 MHz, Benzene-d₆) δ 8.45* (ddd, J=7.1, 5.1, 1.6 Hz, 0.4H), 8.25 (ddd, J=7.2, 5.1, 1.8 Hz, 1H), 7.29 (td, J=7.5, 3.7 Hz, 1.4H), 7.25-7.18 (m, 3H), 7.15-7.10 (m, 2.4H), 7.05* (s, 0.4H), 7.01 (s, 1H), 6.95-6.84 (m, 2.8H), 2.23* (s, 1H), 2.16 (d, J=2.9 Hz, 3H), 2.07* (s, 1H), 2.04* (s, 1H), 2.02 (s, 3H), 2.00 (d, J=3.3 Hz, 3H), 1.25 (d, J=5.9 Hz, 9H), 1.23-1.21 (d, J=5.9 Hz, 9H), 1.20* (d, J=2.5 Hz, 3H), 1.18* (d, J=2.5 Hz, 3H). ¹³C NMR (151 MHz, Benzene-d₆) δ 164.10, 142.88, 140.33, 137.72, 137.08, 135.32, 135.05, 134.40, 130.15, 129.80, 129.33, 129.27, 129.08, 128.82, 128.68, 128.31, 127.13, 122.93, 40.75 (dd, J=20.3, 8.3 Hz), 28.17 (dd, J=14.1, 4.3 Hz), 21.40, 21.33, 19.66 (major isomer only). ³¹P NMR (243 MHz, Benzene-d₆) δ 205.97, 204.95*. * The compound exists as cis and trans isomers; resonances of the minor isomer exists in approx. 1/3 the intensity of the major isomer.

### Synthesis [Pd(1,2-iPr*Phinite)Cl]₂

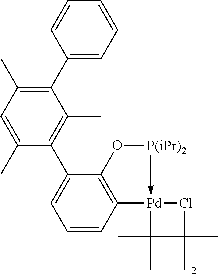

[Pd(1,2-iPr-*Phinite)Cl]₂. The procedure used was the same as [Pd(1,2-tBu*Phinite)Cl]₂ with the exception of the usage of 849.5 mg (2.1 mmol) of 1,2-iPr-*Phinite as starting material (894 mg, 41%). ¹H NMR (600 MHz, Benzene-d₆) δ 8.43* (t, J=6.4 Hz, 0.4H), 8.22 (t, J=7.5 Hz, 1H), 7.22 (m, 3.3H), 7.10 (m, 4.2H), 7.03* (s, 0.4H), 6.99 (s, 1H), 6.95-6.85 (m, 3H), 2.24* (d, J=2.3 Hz, 1H), 2.18 (d, J=3.2 Hz, 3H), 2.08* (d, J=2.4 Hz, 1H), 2.03* (s, 1H), 2.02 (d, J=3.0 Hz, 3H), 2.01 (s, 3H), 1.85 (m, 3H), 1.28-1.07 (m, 9H), 0.93 (m, 9H). ¹³C NMR (151 MHz, Benzene-d₆) δ 162.5, 142.78, 140.35, 137.59, 136.89, 136.77, 135.51, 135.23, 134.55, 130.21, 129.80, −129.43, 129.27, 129.09, 129.03, 128.82, 127.11, 123.13, 29.90 (d, J=28.5 Hz), 21.31 (d, J=8.2 Hz), 19.63, 17.94 (d, J=11.8 Hz), 17.11, 16.94 (major isomer only). ³¹P NMR (243 MHz, Benzene-d₆) δ 200.14, 198.72*. * The compound exists as cis and trans isomers; resonances of the minor isomer exists in approx. 1/3 the intensity of the major isomer.

### Synthesis [Pd(1,2-tBu*Phinite-TIP)Cl]₂

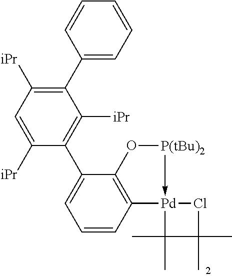

[Pd(1,2-tBu*Phinite-TIP)Cl]₂. The procedure used was the same as [Pd(1,2-tBu*Phinite)Cl]₂ with the exception of the usage of 1.08 g (2.1 mmol) of 1,2-tBu*Phinite-TIP as starting material (1.05 g, 40%). ¹H NMR (600 MHz, Chloroform-d) δ 7.78 (ddd, J=7.4, 5.0, 2.3 Hz, 0.5H), 7.59 (ddd, J=7.1, 5.0, 1.7 Hz, 0.5H), 7.37-7.24 (m, 4H), 7.11 (d, J=2.3 Hz, 2H), 6.86-6.75 (m, 2H), 2.80 (sep, J=7.0, 1H), 2.51 (sep, J=6.5 Hz, 1H), 2.44 (sep, J=6.8 Hz, 1H), 1.38 (m, 18H), 1.16 (t, J=7.4 Hz, 3H), 1.06 (d, J=6.8 Hz, 3H), 1.01 (d, J=6.9 Hz, 3H), 1.00-0.96 (m, 3H), 0.85 (m, 3H), 0.56-0.48 (m, 3H). ¹³C NMR (151 MHz, Chloroform-d) δ 163.24 (d, J=25.8 Hz), 146.95, 146.15, 142.62 (d, J=6.1 Hz), 141.85, 137.47, 136.05, 135.49, 135.13, 131.81, 131.15, 129.49, 127.36, 127.11, 126.54, 121.59, 121.34, 119.13, 40.68 (d, J=19.8 Hz), 40.42 (d, J=20.0 Hz), 32.27, 30.75, 29.68, 28.11, 25.93, 24.56, 24.42, 24.14, 23.41, 23.28. ³¹P NMR (243 MHz, Chloroform-d) δ 206.26, 206.0.

### Synthesis Pd(1,2-tBu*Phinite)(PCy₃)Cl

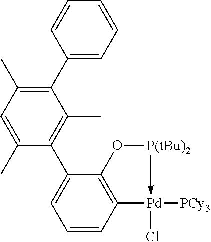

Pd(1,2-tBu*Phinite)(PCy₃)Cl. An oven-dried flask fitted with a magnetic stir-bar was charged with 574 mg (0.5 mmol) of [Pd(1,2-tBu*Phinite)Cl]₂, 280.7 mg of PCy₃ (1.0 mmol) and 15 mL of CH₃CN. The pale yellow suspension was stirred at 25° C. for 16 h to afford an off-white suspension to which was diluted with 15 mL of pentane. The solid was collected by vacuum filtration and washed with pentane to yield a pale yellow powder (692 mg, 81%). ¹H NMR (600 MHz, Benzene-d₆) δ 7.62 (m, 1H), 7.32 (t, J=7.6 Hz, 1H), 7.23 (dd, J=15.5, 7.8 Hz, 2H), 7.15-7.12 (m, 2H), 7.05 (s, 1H), 6.96 (m, 2H), 2.75 (q, J=11.6 Hz, 3H), 2.24 (s, 3H), 2.14 (m, 6H), 2.05 (s, 6H), 1.70 (m 12H), 1.60 (d, J=13.0 Hz, 3H), 1.44 (dd, J=14.3, 4.3 Hz, 18H), 1.25 (m, 6H), 1.15 (m, 3H). ¹³C NMR (151 MHz, Benzene-d₆) δ 165.61 (d, J=12.7 Hz), 143.06, 140.53 (d, J=8.8 Hz), 140.39, 138.67, 137.78, 135.23, 134.81, 134.32, 130.10, 129.90, 129.27, 129.12, 128.81, 127.70, 127.09, 126.01 (d, J=14.6 Hz), 121.74 (d, J=4.4 Hz), 41.40 (dd, J=12.3, 5.3 Hz), 33.12 (d, J=14.3 Hz), 31.19, 28.89 (dd, J=17.0, 4.7 Hz), 28.48 (d, J=10.4 Hz), 27.25, 21.44, 21.38, 19.70. ³¹P NMR (243 MHz, Benzene-d₆) δ 192.22 (d, J=386.8 Hz), 23.42 (d, J=386.5 Hz).

### Synthesis Pd(1,2-iPr*Phinite)(PCy₃)Cl

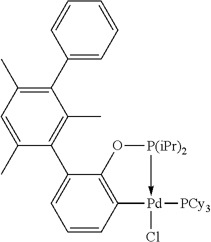

Pd(1,2-iPr-*Phinite)(PCy₃)Cl. The procedure used was the same as Pd(1,2-tBu*Phinite)(PCy₃)Cl with the exception of the usage of 546 mg (0.5 mmol) of [Pd(1,2-iPr-*Phinite)Cl]₂ as the starting material (703 mg, 85%). ¹H NMR (600 MHz, Benzene-d₆) δ 7.63-7.59 (m, 1H), 7.25 (t, J=7.4 Hz, 1H), 7.22-7.18 (m, 2H), 7.14-7.09 (m, 2H), 7.04-6.95 (m, 3H), 2.69 (m, 3H), 2.39 (m, 1H), 2.30 (m, 1H), 2.22 (s, 3H), 2.16-2.10 (m, 6H), 2.05 (s, 3H), 2.04 (s, 3H), 1.75-1.65 (m, 12H), 1.61 (d, J=13.1 Hz, 3H), 1.44 (ddd, J=29.5, 17.9, 7.2 Hz, 6H), 1.32-1.22 (m, 6H), 1.19-1.09 (m, 6H), 1.06 (dd, J=14.7, 7.0 Hz, 3H). ¹³C NMR (151 MHz, Benzene-d₆) δ 165.01 (d, J=14.1 Hz), 142.97, 140.62 (d, J=9.2 Hz), 140.30, 138.56, 137.48, 135.40, 134.85, 134.47, 130.17, 129.91, 129.36, 129.22, 129.08, 128.81, 127.05, 126.03 (d, J=15.5 Hz), 121.91 (d, J=3.8 Hz), 32.83 (d, J=13.7 Hz), 31.12, 29.91 (ddd, J=40.4, 23.6, 4.2 Hz), 28.44 (d, J=10.1 Hz), 27.25, 21.37, 19.67, 18.79 (dd, J=26.5, 4.4 Hz), 17.58, 17.09. ³¹P NMR (243 MHz, Benzene-d₆) δ 189.86 (d, J=391.6 Hz), 22.97 (d, J=391.5 Hz).

### Synthesis Pd(1,3-tBu*Phinite)(py)Cl

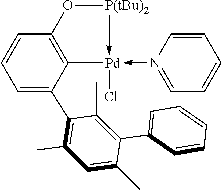

Pd(1,3-tBu*Phinite)(py)Cl. In an oven dried 5 mL microwave vial equipped with a magnetic stir bar, 0.446 g of (1 mmol) of 1,3-tBu*Phinite, 0.177 g (1 mmol) of palladium dichloride, 79 mg (1 mmol) of pyridine, 0.303 g (3 mmol) of triethylanime and dry toluene (1 mL) was added in into the vial in the glove box. The vial was seal under argon and allowed to stir at 100° C. for 18 h. The mixture will then turn black. The mixture was filter through celite in the glove box and the solvent was removed. The crude purified by re-crystallization with dry methanol/hexane mixture to afford a yellow solid (300 mg, yield 45%). ¹H (600 MHz, CDCl₃) δ: 8.28 (d, ³J_{H,H}=3.9 Hz, 2H), 7.58 (t, ³J_{H,H}=6 Hz, 1H), 7.46 (s, 1H), 7.38 (d, ³J_{H,H}=7.2 Hz, 1H), 7.32 (t, ³J_{H,H}=7.32 Hz, 1H), 7.09-7.04 (m, 4H), 6.88 (d, ³J_{H,H}=6.66 Hz, 1H), 6.41 (d, ³J_{H,H}=7.44 Hz, 2H), 2.33 (s, 3H), 2.10 (s, 3H), 1.83 (s, 6H), 1.59 (d, 3J_{H,H}=15.24 Hz, 9H), 1.49 (d, ³J_{H,H}=14.72 Hz, 9H). ¹³C (150 MHz, CDCl₃) δ: 150.03, 136.16, 128.41, 127.60, 125.42, 123.44, 22.08, 20.47, 17.90. ³¹P {¹H} (161 MHz, C₆D₆) δ: 202.18.

### Synthesis Pd(1,3-tBu*Phinite)(py)OAc

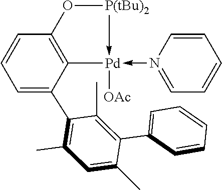

Pd(1,3-tBu*Phinite)(py)OAc. In an oven dried 5 ml microwave vial equipped with a magnetic stir bar 0.446 g of (1 mmol) of 1,3-tBu*Phinite, 0.224 g (1 mmol) of padallium acteate, 79 mg (1 mmol) of pyridine, 0.303 g (3 mmol) of triethylanime and 1 ml of dry toluene was added into the vial in the glove box. The vial was then seal under argon and allowed to stir at 100° C. for 18 h. The reaction mixture was then filter through celite in the glove box and the solvent was removed. The crude purified by re-crystallization with dry methanol/hexane mixture to afford a yellow solid (290 mg, yield 45%). ¹H (600 MHz, C₆D₆) δ: 8.45 (s, 2H), 7.48 (s, 2H), 7.20 (d, ³J_{H,H}=7.32 Hz, 2H), 7.08 (s, 1H), 6.82-6.80 (m, 2H), 6.58 (s, 2H), 6.45 (s, 2H), 2.29 (s, 3H), 2.24 (s, 3H), 2.20 (s, 3H), 1.95 (s, 3H), 1.92 (s, 3H), 1.35 (d, ³J_{H,H}=14.7 Hz, 9H), 1.27 (d, ³J_{H,H}=14.7 Hz, 9H). ¹³C (150 MHz, CDCl₃) δ: 165.508, 165.04, 149.84, 146.52, 141.90, 138.15, 136.15, 134.79, 133.73, 129.32, 128.43, 128.35, 128.20, 128.18, 127.78, 126.20, 124.80, 123.60, 118.87, 118.77, 39.711, 39.71, 39.63, 39.57, 28.13, 28.02, 27.55, 27.10, 20.65, 20.09, 17.56. ³¹P {¹H} (161 MHz, C₆D₆) δ: 200.5.

### Applied Examples of Catalysts

### Mizoroki-Heck Reaction (Alkenylation)

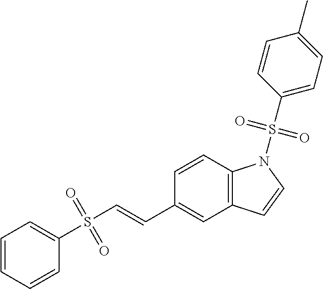 [SMILES: Cc1ccc(S(=O)(=O)n2ccc3cc(/C=C/S(=O)(=O)c4ccccc4)ccc32)cc1]

(E)-5-(2-(phenylsulfonyl)vinyl)-1-tosyl-1H-indole. To a stirred solution of phenyl vinyl sulfone (80 mg, 0.48 mmol, 1.2 equiv) and 5-bromo-1-tosyl-1H-indole (140 mg, 0.4 mmol, 1.0 equiv) in 0.80 mL MeCN was added TIP-Cy*Phine (13.3 mg, 0.024 mmol, 0.06 equiv), palladium diacetate (2.7 mg, 0.012 mmol, 0.03 equiv) and triethylamine (134 μL, 0.96 mmol, 2.4 equiv). The mixture was sealed in a tube and heated at 100° C. for 16 hours. The reaction mixture was cooled to room temperature and filtered. The filtrate was evaporated to obtain the crude product which was purified using flash column chromatography (hexanes/ethyl acetate 2:1, R_{f}=0.25) giving a white solid (122.5 mg, 70%). ¹H NMR (600 MHz, CDCl3) δ 7.98 (d, J=8.7 Hz, 1H), 7.96-7.89 (m, 2H), 7.77-7.70 (m, 3H), 7.65 (d, J=1.7 Hz, 1H), 7.64-7.58 (m, 2H), 7.56-7.51 (m, 2H), 7.43 (dd, J=8.7, 1.8 Hz, 1H), 7.23 (dd, J=8.7, 0.8 Hz, 2H), 6.83 (d, J=15.4 Hz, 1H), 6.66 (dd, J=3.7, 0.8 Hz, 1H), 2.34 (s, 3H). 13C NMR (151 MHz, CDCl3) δ 145:39, 142.70, 140:85, 136.12, 134.94, 133.30, 131.18, 130.02, 129.72, 129.30, 128.06, 127.71, 127.67, 127.57, 126.79, 126.26, 124.34, 122.67, 114.08, 108.98, 21.58. LC-MS (ESI) calcd for C23H19NO4S2Na m/z [M+Na]+: 460.06. found: 460.18.

### Suzuki-Miyaura Cross-Coupling (Arylation)

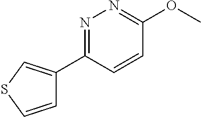

3-methoxy-6-(thiophen-3-yl)pyridazine. In open air, a flask fitted with a magnetic stir bar was charged with 3-thiophene boronic acid (192 mg, 1.5 mmol), 3-chloro-6-methoxypyridazine (144 mg, 1 mmol), KOH (112 mg, 2 mmol), Pd(TIP-Cy*Phine)₂Cl₂ (11 mg, 0.01 mmol) and 1.0 ml of technical grade EtOH. The reaction vessel was sealed with a Teflon-lined cap and mixture was heated to 70° C. with vigorous stirred for 16 h. Upon completion, as judged by GC-MS and TLC, the reaction mixture was filtered through Celite and the filtrate was concentrated under vacuum. The resulting crude residue was purified via column chromatography on silica gel using Hexane/EtOAc (10:1) as the eluent to yield the pure product (161 mg, 84%). ¹H NMR (600 MHz, Chloroform-d) δ 7.87 (dd, J=3.0, 1.3 Hz, 1H), 7.76 (dd, J=5.0, 1.3 Hz, 1H), 7.68 (d, J=9.2 Hz, 1H), 7.42 (dd, J=5.1, 3.0 Hz, 1H), 7.00 (d, J=9.1 Hz, 1H), 4.16 (s, 3H). ¹³C NMR (151 MHz, Chloroform-d) δ 164.00, 151.85, 138.70, 127.18, 126.88, 126.06, 123.49, 117.92, 54.97.

### Buchwald-Hartwig Amination

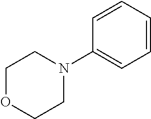 [SMILES: c1ccc(N2CCOCC2)cc1]

4-Phenylmorpholin. To a flask fitted with a magnetic stir bar was added 1.8 mg (0.01 mmol) of PdCl₂, 11.1 mg (0.02 mmol) of TIP-Cy*Phine, 0.105 mL (1.2 mmol) of morpholine, 0.102 mL (1.0 mmol) of chlorobenzene, 134.5 mg (1.4 mmol) of NaOtBu and 0.75 mL of THF. The flask was sealed under air and the mixture was stirred at 80° C. for 22 h. The mixture then cooled to room temperature and diluted with dichloromethane. The resulting solution was filtered through a pad of Celite and concentrated to dryness under reduced pressure. The crude product was purified by silica gel column chromatography using a hexanes/EtOAc (92:8) mixture as the eluent to afford the pure white product (161 mg, 99%). ¹H NMR (400 MHz, CDCl₃): δ 7.30-7.24 (m, 2H), 6.93-6.86 (m, 3H), 3.84 (t, J=4.7 Hz, 4H), 3.13 (t, J=4.7 Hz, 4H). ¹³C NMR (100 MHz, CDCl₃): δ 151.8, 129.7, 120.5, 116.2, 67.4, 49.8.

### Copper-Free Sonogashira Cross-Coupling (Alkynylation)

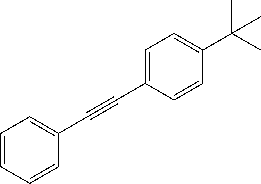 [SMILES: CC(C)(C)c1ccc(C#Cc2ccccc2)cc1]

1-(tert-butyl)-4-(phenylethynyl)benzene. To a sealable reaction tube equipped with a stirring bar was charged with PdCl₂(CH₃CN)₂ (2.6 mg, 0.01 mmol), TIP-Cy*Phine (11.1 mg, 0.02 mmol), Cs₂CO₃ (652 mg, 2 mmol), 1-tert-butyl-4-chlorobenzene (169 mg, 1 mmol), phenylacetylene (123 mg, 1 mmol) and 2 mL of acetonitrile. The tube was then sealed with a Teflon-lined septum and heated to 90° C. for 6 h with vigorous stirring. The resulting suspension was cooled to room temperature, diluted with EtOAc and filtered through a pad of Celite. The filtrate was concentrated in vacuo affording the crude product which was purified by flash chromatography on silica gel using hexanes as the eluent to give a pure white solid (213 mg, 91%). ¹H NMR (600 MHz, CDCl₃) δ=7.54 (d, J=5.8 Hz, 2H), 7.49 (d, J=8.5 Hz, 2H), 7.37 (dd, J=21.8, 7.6 Hz, 5H), 1.35 (s, 9H). ¹³C NMR (151 MHz, CDCl₃) δ=152.08, 132.13, 131.88, 128.86, 128.61, 125.90, 124.07, 120.79, 90.09, 89.28, 35.35, 31.75.

### Kumada-Tamao-Corriu Cross-Coupling

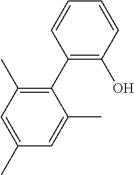 [SMILES: Cc1cc(C)c(-c2ccccc2O)c(C)c1]

2′,4′,6′-trimethyl-[1,1′-biphenyl]-2-ol. In a sealable reaction tube equipped with a stirring bar, NaH (25.2 mg, 1.05 mmol) was suspended in 0.3 mL of THF. At room temperature, 173 mg (1 mmol) of 2-bromophenol dissolved in 0.25 mL of THF was added to the suspension dropwise. After the effervescence had ceased, Pd₂(dba)₃ (9.2 mg, 0.01 mmol) and TIP-Cy*Phine (22.1 mg, 0.04 mmol) was added to the mixture. In a separate flask, 2,4,6-trimethylphenyl magnesium bromide was prepared by stirring 298.6 mg (1.5 mmol) of 2,4,6-trimethylphenyl bromide with 48.6 mg (2 mmol) of activated Mg turnings in 0.5 mL of THF at room temperature for 16 h. Upon formation of the Grignard solution, the reagent was rapidly added via syringe to the other reaction tube containing the catalyst mixture. The resultant yellow-brown suspension was stirred at 70° C. for 16 h. After the reaction was complete, as determined by GC-MS, the resultant grey suspension was cooled to room temperature and quenched with 2 mL of a 0.5 M aq Na₃EDTA solution. The crude product was extracted with 3×5 mL of EtOAc. The organic portions were combined, washed with H₂O and brine, dried with MgSO₄, filtered and concentrated in vacuo affording viscous yellow oil. The crude product was purified by flash chromatography on silica gel using petroleum ether/EtOAc (0 to 20% EtOAc) as the column eluent to yield an off-white solid (148 mg, 70%). ¹H NMR (600 MHz, Chloroform-d) δ 7.27 (ddd, J=8.0, 7.1, 2.0 Hz, 1H), 7.02-6.95 (m, 5H), 4.65 (s, 1H), 2.33 (s, 3H), 2.01 (s, 6H). ¹³C NMR (151 MHz, Chloroform-d) δ 152.62, 138.27, 137.99, 131.80, 130.22, 129.09, 128.93, 126.58, 120.89, 115.26, 21.30, 20.43.

### Aryl Fluorination

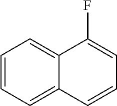 [SMILES: Fc1cccc2ccccc12]

1-fluoronapthalene. To an oven-dried flask equipped with a magnetic stir bar was added 3.9 mg (0.0075 mmol) of [Pd(cinnamy)Cl]₂, 8.4 mg (0.015 mmol) of 25OMe-TIP-tBu*Phine, 151.9 mg (1.0 mmol) of CsF, 138.1 mg (0.5 mmol) of 1-naphthyl-triflate and 2.5 mL of anhydrous toluene. The flask was crimp sealed with a Teflon-lined cap under argon, heated to 100° C. in an oil bath and vigorously stirred for 18 h. Upon reaction completion, the mixture was cooled to room temperature, diluted with EtOAc and filtered through a pad of Celite. Several portions of EtOAc were used to rinse the Celite and the filtrate was consolidated then concentrated to dryness. The crude residue was purified by column-chromatography on silica gel using petroleum ether as the eluent to give the pure product as a colorless liquid (56 mg, 76%). ¹H NMR (600 MHz, Chloroform-d) δ 8.18-8.11 (m, 1H), 7.91-7.85 (m, 1H), 7.65 (d, J=8.3 Hz, 1H), 7.59-7.53 (m, 2H), 7.41 (td, J=8.0, 5.4 Hz, 1H), 7.17 (ddd, J=10.7, 7.6, 1.0 Hz, 1H). ¹³C NMR (151 MHz, Chloroform-d) δ 159.59, 134.87, 127.51, 127.48, 126.79, 126.15, 126.14, 125.60, 125.54, 123.63, 123.60, 120.53, 120.49, 109.45, 109.32. ¹⁹F NMR (280 MHz, Chloroform-d) δ−124.6.

1. A compound of structure I:
(Ar¹—Ar²—Ar³-E-P(=D)R₂—-)ₙMₘX_{n′}L_{n″}
wherein:
Ar¹, Ar² and Ar³ are aromatic groups wherein:
Ar¹ and Ar³ are in a 1,3 relationship on Ar²,
each of Ar¹, Ar² and Ar³ optionally comprises one or more ring substituents of formula YR′ᵣ wherein each Y independently is absent or is O, S, B, N or Si and each R′ is independently H, halogen, alkyl, cycloalkyl, aryl or heteroaryl and r is 1, 2 or 3, where r is 1 if Y is absent or is O or S, 2 if Y is B or N and 3 if Y is Si,
Ar¹, Ar² and Ar³ are each independently carbocyclic or heterocyclic and each is independently monocyclic, bicyclic or polycyclic and each ring of each of Ar¹, Ar² and Ar³ independently has 5, 6 or 7 ring atoms;
E is absent or is selected from the group consisting of O, S, NR″, SiR″₂, AsR″₂ and CR″₂;
M is a complexing metal;
X is selected from the group consisting of H, F, Br, Cl, I, OTf, dba (dibenzylidene acetone), OC(═O)CF₃ and OAc;
L is absent or is selected from the group consisting of PR″₂, NR″₂, OR″, SR″, SiR″₃, AsR″₃, alkene, alkyne, aryl and heteroaryl, each of said alkene, alkyne, aryl and heteroaryl being optionally substituted, for example with one or more halogens and/or with one or more R groups as defined herein, wherein if L is absent, n is not 1;
each R is independently alkyl, cycloalkyl, heterocyclyl, heterocycloalkyl, aryl, heteroaryl, alkyloxy, cycloalkyloxy, heterocyclyloxy, heterocycloalkyloxy, aryloxy or heteroaryloxy;
D is absent or is ═S or ═O or —Z-linker-Z—, where each Z independently is O or NH or N-alkyl and linker is an alkyl chain of 2-5 carbon atoms in length;
each R″ is independently H, alkyl, cycloalkyl, heterocycloalkyl, aryl or heteroaryl, each other than H being optionally substituted, or R″₂ is —Z-linker-Z— as defined above; and
m is 0 or 1 or 2; wherein:
if m is 0, n is 1, n′ and n″ are 0 and -- is absent; and
if m is 1 or 2, n is 1 or 2 and n′ and n″ are integers such that the coordination sphere of M is filled, and D is absent.

2. The compound of claim 1 wherein m is 1 or 2 and M is bonded to a ring atom of Ar³ ortho to E.

3. The compound of claim 1 or claim 2 wherein E is O or S or NR″.

4. The compound of any one of claims 1 to 3 wherein n is 1 or 2 and M is selected from the group consisting of Pd, Ni, Pt, Cu, Fe, Co, Au, Ag, Rh, Ir, Ru, Os or Mn.

5. The compound of claim 4 wherein M is Pd, Ni or Cu.

6. The compound of claim 5 wherein M is Pd.

7. The compound of any one of claims 1 to 6 wherein m is 1 or 2 and X is Cl or OAc.

8. The compound of claim 1 wherein m=0.

9. The compound of claim 1 wherein m=1.

10. The compound of claim 1 wherein m=2 and n″ is 0.

11. The compound of any one of claims 1 to 7, 9 or 10 wherein n=m.

12. The compound of any one of claims 1 to 11 wherein each R is independently selected from the group consisting of alkyl, cycloalkyl, aryl and heteroaryl.

13. The compound of claim 12 wherein each R is alkyl or each is aryl, or one is alkyl and the other is aryl.

14. The compound of any one of claims 1 to 7 or 9 to 12 wherein n″ is 0, M is Pd and n is 2.

15. The compound of claim 1 wherein Ar¹ is phenyl, Ar² is 2,4,6-trimethylphenyl or 2,4,6-triisopropylphenyl, Ar³ is phenyl, E is O, R attached to P is each alkyl or each is aryl, or one is alkyl and the other is aryl, and n is 1 and either m, n′ and n″ are all 0 or m is 1, n′ is 1 and n″ is 0 and M is Pd.

16. The compound of claim 1 wherein if E is absent, n is 1 and m, n′ and n″ are all 0.

17. A process for making a compound of structure I as defined in claim 1, said process comprising reacting a compound of structure II:
Ar¹—Ar²—Ar³-E-PR₂
wherein Ar¹, Ar² and Ar³, E and R are as defined in claim 1 and Ar¹ and Ar³ are in a 1,3 relationship on Ar²,
with a salt or complex of M, optionally in the presence of a liganding species.

18. The process of claim 17 wherein no liganding species is present, whereby m=2, said process comprising the subsequent step of exposing the compound of structure I in which m=2 to a liganding species so as to produce a compound of structure I in which m=1 and L is said liganding species or is derived therefrom.

19. The process of claim 17 or claim 18 wherein the liganding species is a phosphine, an amine, an alcohol, a thiol, a silane, an arsine, an olefin, an aromatic compound or a heteroaromatic compound.

20. The process of any one of claims 17 to 19 wherein M is selected from the group consisting of Pd, Ni, Pt, Cu, Fe, Co, Au, Ag, Rh, Ir, Ru, Os and Mn.

21. The process of any one of claims 17 to 20 wherein the salt or complex of M is a dba, diene, olefin, allyl, silane, nitrile or organonitrile complex (such as Pd(PhCN)₂Cl₂, Pd(CN)₂ or Pd(MeCN)₄(BF₄)₂ or Pd(COD)(CH₂TMS)₂ or Pd(NBD)(CH₂TMS)₂), or a halide or acetate or cyanide salt or a salt of a pseudohalide, e.g. triflate, of M.

22. The process of claim 20 wherein M is Pd, Ni or Cu.

23. The process of claim 22 wherein M is Pd.

24. The process of any one of claims 17 to 23 comprising reacting a compound of structure III:
Ar¹—Ar²—Ar³-E-X
wherein Ar¹, Ar² and Ar³ and E are as defined in claim 1 and Ar¹ and Ar³ are in a 1,3 relationship on Ar²,
with either:
a compound of structure X—PR₂, wherein R is as defined in claim 1 or
a compound of structure X—P(D)R₂ wherein R is as defined in claim 1, and subsequently removing the group D, wherein D is ═S or ═O or —Z-linker-Z—, where each Z independently is O or NH or N-alkyl and linker is an alkyl chain of 2-5 carbon atoms in length
wherein each X is independently selected from the group consisting of H, F, Br, Cl, I, OTf, dba, OC(═O)CF₃ and OAc
so as to produce the compound of structure II.

25. A process for making a compound of structure II as defined in claim 17 comprising reacting a compound of structure III:
Ar¹—Ar²—Ar³-E-X
wherein Ar¹, Ar² and Ar³ and E are as defined above and Ar¹ and Ar³ are in a 1,3 relationship on Ar²,
with either:
a compound of structure X—PR₂, wherein R is as defined in claim 1 or
a compound of structure X—P(D)R₂ wherein R is as defined in claim 1, and subsequently removing the group D, wherein D is ═S or ═O or —Z-linker-Z—, where each Z independently is O or NH or N-alkyl and linker is an alkyl chain of 2-5 carbon atoms in length
wherein each X is independently selected from the group consisting of H, F, Br, Cl, I, OTf, dba, OC(═O)CF₃ and OAc.

26. The process of any one of claims 17 to 25 wherein each ring in Ar¹, Ar² and Ar³ has independently, 5 or 6 ring carbon atoms.

27. The process of any one of claims 17 to 26 wherein at least one of Ar¹, Ar² and Ar³ is a fused aromatic ring or a fused heteroaryl ring.

28. The process of any one of claims 17 to 26 wherein each of Ar¹, Ar² and Ar³ is monocyclic, e.g. is a phenyl ring.

29. The process of any one of claims 17 to 28 wherein E is O, S, NR″₂ or absent.

30. The process of any one of claims 17 to 29 wherein the R groups on P are both alkyl or both are aryl or one R is alkyl and one is aryl.

31. The process of claim 24 or 25 comprising the step of conducting a metal mediated cross-coupling reaction of a biaryl compound with an aryl compound so as to produce the compound of structure III.

32. The process of claim 31 comprising reacting a compound of structure IV: Ar¹—Ar²A in which A is a leaving group or activating group, e.g. a halogen or a triflate group or a boronic acid or boronic ester, and in which A is meta to Ar¹ on Ar², with a compound of structure XEAr³A or Ar³A in the presence of an electrophile, so as to produce the compound of structure III.

33. The process of claim 32 wherein A is either an electron donating group or an electron withdrawing group.

34. The process claim 17 comprising:
providing a 2,4,6-trialkylbiphenyl
halogenating said 2,4,6-trialkylbiphenyl in the 3 position
metallating the resulting halogenated biphenyl and reacting the resulting intermediate with an o-halophenol to form a 2-hydroxy-2′,4′,6′-trialkyl-m-terphenyl, or with an o-dihalobenzene to form a 2-halo-2′,4′,6′-trialkyl-m-terphenyl, and
reacting the m-terphenyl with a base and a halodialkylphosphine or halodiarylphosphine or haloalkylarylphosphine to produce a dialkylphosphinite or diarylphosphinite or alkylarylphosphinite of 2-hydroxy-2′,4′,6′-trialkyl-m-terphenyl respectively or else metallating the m-terphenyl and reacting the resulting intermediate, optionally in the presence of a copper salt and/or lithium salt, and a halodialkylphosphine or halodiarylphosphine or haloalkylarylphosphine to produce a dialkylphosphine or diarylphosphine or alkylarylphosphine respectively, each of said dialkylphosphinite or diarylphosphinite or alkylarylphosphinite or dialkylphosphine or diarylphosphine or alkylarylphosphine being a compound of structure II.

35. The process of claim 34 wherein, in the step of metallating, the intermediate is reacted with an o-halophenol and the subsequent step comprises reacting the resulting m-terphenyl with a base and a halodialkylphosphine or halodiarylphosphine or haloalkylarylphosphine to produce a dialkylphosphinite or diarylphosphinite or alkylarylphosphinite of 2-hydroxy-2′,4′,6′-trialkyl-m-terphenyl respectively, each being of structure II in which E is O.

36. The process of claim 35 comprising the subsequent step of reacting the compound of structure II with a palladium halide so as to form a compound of structure I as defined in claim 1 in which E is O, m, n and n′ are all 2, n″ is 0 and both R groups are alkyl or both are aryl or one R is alkyl and the other is aryl.

37. The process of claim 35 comprising reacting the compound of structure II with a palladium halide in the presence of a phosphine ligand whereby the process forms a compound of structure I as defined in claim 1 in which E is O, m, n and n′ are all 1, n″ is 1 and both R groups are alkyl or both are aryl or one R is alkyl and the other is aryl and L is the phosphine ligand.

38. The process of claim 36 comprising the subsequent step of reacting the compound of structure I with a phosphine ligand, e.g. PCy₃, so as to produce a compound of structure I as defined in claim 1 in which E is O, m, n and n′ are all 1, n″ is 1 and both R groups are alkyl or both are aryl or one R is alkyl and the other is aryl and L is the phosphine ligand, e.g. PCy₃.

39. The process of any one of claims 17 to 38 which is conducted as a one pot process without isolation of intermediates or in which any two or more contiguous steps are conducted as a one pot process.

40. The process of any one of claims 17 to 39 which is conducted as a continuous reaction or in which any one or more individual steps thereof is (are) conducted as a continuous reaction.

41. A process for making a coupled compound comprising reacting a compound of structure R^{a}X in which R^{a} is alkyl, alkenyl, alkynyl, aryl or heteroaryl and X is a leaving group such as halide or triflate, with a compound V comprising an element M bonded directly to an alkyl, alkenyl, alkynyl, aryl or heteroaryl group, in which M is an element in one of groups 1, 2, 11, 12, 13 or 14, in the presence of either:
a compound of structure I as defined in claim 1 in which m=1 or 2 and M=Pd, or
a compound of structure I as defined in claim 1 in which m=0 and a palladium salt or complex capable of reacting with the compound of structure I to form a compound of structure I in which m=1 or 2 and M=Pd.

42. The process of claim 41 wherein either R^{a} or compound V or both is substituted with one or more substituents.

43. The process of claim 42 wherein each of the one or more substituents is, independently, alkyl, aryl, heteroaryl; halogen, OR^{b}, NR^{b}R^{c}, BR^{b}R^{c}, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl.

44. The process of claim 43 wherein compound V is a boronic acid.

45. A process for making a olefinic compound comprising reacting a compound of structure R^{a}X in which R^{a} is alkyl, alkenyl, alkynyl, aryl or heteroaryl and X is a leaving group such as halide or triflate with an olefin in the presence of either:
a compound of structure I as defined in claim 1 in which m=1 or 2 and M=Pd, or
a compound of structure I as defined in claim 1 in which m=0 and a palladium salt or complex capable of reacting with the compound of structure I to form a compound of structure I in which m=1 or 2 and M=Pd.

46. The process of claim 45 wherein either R^{a} or the olefin or both is substituted with one or more substituents.

47. The process of claim 46 wherein each of the one or more substituents is, independently, alkyl, aryl, heteroaryl, halogen, OR^{b′}, NR^{b′}R″, BR^{b}R″, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl group.

48. A process for making an alkynyl compound comprising reacting a compound of structure R^{a}X in which R^{a} is alkyl, alkenyl, alkynyl, aryl or heteroaryl and X is a leaving group such as halide or triflate with a terminal alkyne in the presence of either:
a compound of structure I as defined in claim 1 in which m=1 or 2 and M=Pd, or
a compound of structure I as defined in claim 1 in which m=0 and a palladium salt capable of reacting with the compound of structure I to form a compound of structure I in which m=1 or 2 and M=Pd.

49. The process of claim 48 wherein R^{a} is substituted with one or more substituents.

50. The process of claim 49 wherein each of the one or more substituents is, independently, alkyl, aryl, heteroaryl, halogen, OR^{b}, NR^{b}R^{c}, BR^{b}R^{c}, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl.

51. A process for making an amine compound comprising reacting a compound of structure R^{a}X in which R^{a} is alkyl, alkenyl, alkynyl, aryl or heteroaryl and X is a leaving group such as halide or triflate with a primary or secondary amine in the presence of either:
a compound of structure I as defined in claim 1 in which m= or 2 and M=Pd, or
a compound of structure I as defined in claim 1 in which m=0 and a palladium salt capable of reacting with the compound of structure I to form a compound of structure I in which m=1 or 2 and M=Pd.

52. The process of claim 51 wherein R^{a} is substituted with one or more substituents.

53. The process of claim 52 wherein each of the one or more substituents is, independently, alkyl, aryl, heteroaryl, halogen, OR^{b}, NR^{b}R^{c}, BR^{b}R^{c}, SiR^{b}₃ or SiR^{b}R^{c}₂ or SiR^{b}R^{c}R^{d}, in which R^{b}, R^{c} and R^{d} are each independently H, alkyl, alkenyl, alkynyl, OH, amine, halogen, alkoxyl or aryl.

54. The process of any one of claims 51 to 53 wherein the amine is of the form R^{e}NH₂ or R^{e}R^{f}NH in which R^{e} and R^{f} are, independently, alkyl, alkenyl, heteroaryl or aryl groups.

55. A process for making an aryl carbonyl compound, e.g. a ketone or aldehyde or acid or ester, comprising reacting a compound of structure R^{a}X in which R^{a} is alkyl, alkenyl, alkynyl, aryl or heteroaryl and X is a leaving group such as halide or triflate with carbon monoxide and a nucleophile M′-Nu, where Nu=OH, NR₂, alkyl, aryl, SR, or OR^{a} where each R^{a} is as described above, and M′ is H or a Group 1 or Group 2 (alkyl or alkaline earth) metal ion or is M″R^{b}, where M is Cu, Ag, Zn, AlR, GaR^{b}, TlR^{b}, CR^{b}₂, SiR^{b}₂, SnR^{b}₂, PbR^{b}₂, NR^{b} or AsR^{b}, where R^{b} is alkyl, alkenyl, alkynyl, aryl or heteroaryl, in the presence of either:
a compound of structure I as defined in claim 1 in which m=1 or 2 and M=Pd, or
a compound of structure I as defined in claim 1 in which m=0 and a palladium salt capable of reacting with the compound of structure I to form a compound of structure I in which m=1 or 2 and M=Pd.

56. The process of claim 55 wherein R^{a} is substituted with one or more substituents.

57. The process of claim 56 wherein each of the one or more substituents is, independently, alkyl, aryl, heteroaryl, halogen, OR^{c}, NR^{c}R^{d}, BR^{c}R^{d}, SiR^{c}₃ or SiR^{c}R^{d}₂ or SiR^{c}R^{d}R^{e}, in which R^{c}, R^{d} and R^{e} are each independently H, alkyl, alkenyl, alkynyl, OH, amine, halogen, alkoxyl or aryl.

58. A process for making a nitrile compound comprising reacting a compound of structure R^{a}X in which R is alkyl, alkenyl, alkynyl, aryl or heteroaryl and X is a leaving group such as halide or triflate with a cyanide in the presence of either:
a compound of structure I as defined in claim 1 in which m=1 or 2 and M=Pd, or
a compound of structure I as defined in claim 1 in which m=0 and a palladium salt capable of reacting with the compound of structure I to form a compound of structure I in which m=1 or 2 and M=Pd.

59. The process of claim 58 wherein R is substituted with one or more substituents.

60. The process of claim 59 wherein each of the one or more substituents is, independently, alkyl, aryl, heteroaryl, halogen, OR^{c}, NR^{c}R^{d}, BR^{c}R^{d}, SiR^{c}₃ or SiR^{c}R^{d}₂ or SiR^{c}R^{d}R^{e}, in which R^{c}, R^{d} and R^{e} are each independently H, alkyl, alkenyl, alkynyl, OH, amine, halogen, alkoxyl or aryl.

61. The process of any one of claims 58 to 60 wherein the cyanide is a Group I metal cyanide or HCN.

62. A process for making a phosphorus compound comprising reacting a compound of structure R^{a}X in which R^{a} is alkyl, alkenyl, alkynyl, aryl or heteroaryl and X is a leaving group such as halide or triflate with a phosphorus reagent in the presence of either:
a compound of structure I as defined in claim 1 in which m=1 or 2 and M=Pd, or
a compound of structure I as defined in claim 1 in which m=0 and a palladium salt capable of reacting with the compound of structure I to form a compound of structure I in which m=1 or 2 and M=Pd.

63. The process of claim 62 wherein R^{a} is substituted with one or more substituents.

64. The process of claim 63 wherein each of the one or more substituents is, independently, alkyl, aryl, heteroaryl, halogen, OR^{c}, NR^{c}R^{d}, BR^{c}R^{d}, SiR^{c}₃ or SiR^{c}R^{d}₂ or SiR^{c}R^{d}R^{e}, in which R^{c}, R^{d} and R^{e} are each independently H, alkyl, alkenyl, alkynyl, OH, alkoxyl or aryl.

65. The process of any one of claim 62 to 64 wherein the phosphorus reagent is X—PR^{b}₂ or X₂—PR^{b} where X═H or halide, or X₂ is ═O or ═S or the phosphorus reagent is a protected phosphine such as a borane adduct.

66. A process for making an ether comprising reacting a compound of structure R^{a}X in which R^{a} is alkyl, alkenyl, alkynyl, aryl or heteroaryl and X is a leaving group such as halide or triflate with an alcohol in the presence of either:
a compound of structure I as defined in claim 1 in which m=1 or 2 and M=Pd, or
a compound of structure I as defined in claim 1 in which m=0 and a palladium salt capable of reacting with the compound of structure I to form a compound of structure I in which m=1 or 2 and M=Pd.

67. The process of claim 66 wherein R^{a} is substituted with one or more substituents.

68. The process of claim 67 wherein each of the one or more substituents is, independently, alkyl, aryl, heteroaryl, halogen, OR^{c}, NR^{c}R^{d}, BR^{c}R^{d}, SiR^{c}₃ or SiR^{e}R^{d}₂ or SiR^{c}R^{d}R^{e}, in which R^{c}, R^{d} and R^{e} are each independently H, alkyl, alkenyl, alkynyl, OH, amine, halogen, alkoxyl or aryl.

69. Use of a compound of structure I as defined in claim 1 in which m=1 or 2 and M=Pd as a catalyst.

70. Use according to claim 69, being for catalysis of a reaction selected from the group consisting of Suzuki-Miyaura, Kumada, Stille, Negishi and Hiyama coupling reactions, Heck and Sonogashira reactions, Buchwald-Hartwig amination, Heck carbonylation, alkoxylation, cyanation, phosphination and metal mediated coupling reactions.
